(19) United States
(12) Patent Application Publication
(10) Pub. No.: US 2018/0119122 A1
(43) Pub. Date: May 3, 2018

(54) METHODS AND COMPOSITIONS FOR CELLULAR REPROGRAMMING

(71) Applicants: YouHealth Biotech, Limited, Grand Cayman (KY); The Regents of the University of California, Oakland, CA (US)

(72) Inventors: Kang ZHANG, San Diego, CA (US); Rui HOU, Shenyang (CN); Gen LI, Shenyang (CN)

(21) Appl. No.: 15/803,508

(22) Filed: Nov. 3, 2017

Related U.S. Application Data
(60) Provisional application No. 62/417,194, filed on Nov. 3, 2016.
(60) Provisional application No. 62/479,167, filed on Mar. 30, 2017.

(51) Int. Cl. C12N 9/22; C12N 5/079; C12N 15/85

(52) U.S. Cl. CPC C12N 9/22; C12N 15/8509; C12N 5/0621

(57) ABSTRACT

Disclosed herein are methods and pharmaceutical compositions for the treatment of retinitis pigmentosa, macular degeneration and other retinal conditions by interfering with expression of genes, such as those encoding photoreceptor cell-specific nuclear receptor and neural retina-specific leucine zipper protein, in cells of the eye. These methods and compositions employ nucleic acid based therapies.

### CROSS-REFERENCE

This application claims the benefit of U.S. Provisional Application No. 62/417,194 filed Nov. 3, 2016, and U.S. Provisional Application No. 62/479,167 filed Mar. 30, 2017, which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Oct. 31, 2017, is named 49697-713-SEQ.txt and is 4.31 KB in size.

### BACKGROUND OF THE DISCLOSURE

Gene therapy, delivery of nucleic acids to cells of patients to treat a condition, has been contemplated and tested for decades with varying success. Conditions treated are generally terminal illnesses (e.g., cancer, leukemia) and extremely debilitating diseases (e.g., severe combined immunodeficiency).

### SUMMARY OF THE DISCLOSURE

Disclosed herein are methods of re-programming a cell from a first cell type to a second cell type, comprising contacting the cell with a first guide RNA that hybridizes to a target site of a gene, wherein the gene encodes a protein that contributes to a cell type specific function of the cell; and a Cas nuclease that cleaves a strand of the gene at the target site, wherein cleaving the strand modifies expression of the gene such that the cell can no longer perform the cell type specific function, thereby re-programming the cell to the second cell type. The gene may comprise a mutation. The first cell type may be sensitive to the mutation and wherein the second cell type is a cell type that is resistant to the mutation. The mutation may cause a detrimental effect only in the first cell type. The detrimental effect may be selected from senescence, apoptosis, lack of differentiation, and aberrant cellular proliferation. The gene may encode a transcription factor. The first cell type and the second cell type may be closely related, terminally differentiated mature cell types. The re-programming may occur in vivo. The re-programming may occur in vitro or ex vivo. The cell may be a cell of the pancreas, heart, brain, eye, intestine, colon, muscle, nervous system, prostate or breast. The cell may be a post-mitotic cell. The cell may be a cell in an eye. The cell may be a retinal cell. The retinal cell may be a rod. The cell type specific function may be night vision or color vision. The gene may be selected from NRL, NR2E3, GNAT1, ROR beta, OTX2, CRX and THRB. The gene may be selected from NRL and NR2E3. The first cell type may be a rod and the second cell type may be a cone. The cone may be capable of light vision in a subject. The first cell type may be a rod and the second cell type may be a pluripotent cell. The first cell type may be a rod and the second cell type may be a multi-potent retinal progenitor cell. The cell may be a cancer cell. The function may be selected from aberrant cellular proliferation, metastasis, and tumor vascularization. The first cell type may be a colon cancer cell and the second cell type may be a benign intestinal or colon cell. The gene may be selected from APC, MYH1, MYH2, MYH3, MLH1, MSH2, MSH6, PMS2, EPCAM, POLE1, POLD1, NTHL1, BMPR1A, SMAD4, PTEN, and STK11. The first cell type may be a malignant B cell and the second cell type may be a benign macrophage. The gene may be selected from C-MYC, CCND1, BCL2, BCL6, TP53, CDKN2A, and CD19. The cell may be a neuron. The cell may be an interneuron. The interneuron may be a horizontal cell. The first cell type may produce at least one protein selected from amyloid beta, tau protein, and a combination thereof, and the second cell type may not produce the protein or produces less of the protein than the first cell type. The first cell type may be a neuron and the second cell type may be a glial cell. The gene may be selected from APP and MAPT. The first cell type produces alpha synuclein. The first cell type may be a glial cell and the second cell type may be a dopamine producing neuron. The gene may be selected from SNCA, LRRK2, PARK2, PARK7, and PINK1. The gene may be alpha synuclein (SNCA). The second cell type may be selected from a dopaminergic neuron and a dopaminergic progenitor cell. The first cell type may be a non-dopaminergic neuron or a glial cell.

Further disclosed herein are methods of treating a condition in a subject in need thereof with a re-programmed cell, wherein the re-programmed cell is produced by contacting a cell with a first guide RNA that hybridizes to a target site of a gene, wherein the gene encodes a protein that contributes to a cell type specific function of the cell; and a Cas nuclease that cleaves a strand of the gene at the target site, wherein cleaving the strand modifies expression of the gene such that the cell can no longer perform the cell type specific function, thereby re-programming the cell to the second cell type. The re-programmed cell may be autologous to the subject. The condition may comprise retinal degeneration. The condition may be selected from macular degeneration, retinitis pigmentosa, and glaucoma. The condition may be retinitis pigmentosa. The condition may be cancer. The cancer may be colon cancer or breast cancer. The condition may be a neurodegenerative condition. The condition may be selected from Parkinson's Disease and Alzheimer's Disease.

Disclosed herein are methods of treating a condition comprising administering to a subject in need thereof: a first guide RNA that hybridizes to a target site of a gene in a first type of cell, wherein the gene encodes a protein that contributes to a first function of the first type of cell; and a Cas nuclease that cleaves a strand of the gene at the target site, wherein cleaving the strand modifies expression of the gene such that the first type of cell is switched from a first type of cell to a second type of cell, wherein a resulting presence or increase in the second type of cell improves the condition. Modifying expression of the gene may comprise reducing expression of the gene in the first type of cell by at least about 90%. Modifying expression of the gene may comprise editing the gene, wherein the editing results in production of no protein from the gene or a non-functional protein from the gene. The condition may be an eye condition, and the first type of cell may be a first type of eye cell and the second type of cell is a second type of eye cell. The function may be performed in the first type of eye cell and not in the second type of eye cell. The second type of eye cell may perform a second function, wherein the second function may be not performed by the first type of eye cell. The first type of eye cell may be a rod and the second type of eye cell may be a cone. The eye condition may be retinal degeneration, retinitis pigmentosa or macular degeneration. The gene may be selected from NR2E3 and NRL. The method may comprise re-programming a rod to a cone or a rod to a multi-potent retinal progenitor cell. The eye condition may be glaucoma and the second type of eye cell may be a retinal ganglion cell. The first cell type may be a muller glial cell. The gene may be ATOH7. The gene may be a POU4F gene (POU4F1, POU4F2, or POU4F3), which encodes a BRN-3 protein (BRN3A, BRN3B, BRN3C, respectively). The gene may be Islet1, also referred to as ISL1. The gene may be CDKN2A, which encodes p16. The gene may be Six6. The method may comprise administering at least one polynucleotide encoding the Cas nuclease and the guide RNA in a delivery vehicle selected from a vector, a liposome, and a ribonucleoprotein. The method may comprise contacting the cell with a second guide RNA. The method may comprise administering a second guide RNA. The method may comprise introducing a novel splice site in the gene. Introducing the novel splice site may result in removal of an exon, or portion thereof, from a coding sequence of the gene. The exon may comprise a mutation in the gene. The mutation may cause a detrimental effect only in the first cell type. The detrimental effect may be selected from senescence, apoptosis, lack of differentiation, and aberrant cellular proliferation. The gene may encode a transcription factor. The first type of cell may be sensitive to the mutation and the second type of cell may be resistant to the mutation. The method may comprise introducing a novel exon to the gene. The method may comprise introducing at least one nucleotide to the gene. The method may comprise introducing a novel exon to the gene.

Further disclosed herein are systems comprising a Cas nuclease or a polynucleotide encoding the Cas nuclease, a first guide RNA and a second guide RNA, wherein the first guide RNA targets Cas9 cleavage of a first site 5′ of at least a first region of a gene and the second guide RNA targets Cas9 cleavage of a second site 3′ of the first region of the gene, thereby excising the region of the gene. The first guide RNA may target Cas9 cleavage of a first site 5′ of at least a first exon and the second guide RNA targets Cas9 cleavage of a second site 3′ of at least the first exon, thereby excising the at least first exon. The system may comprise a donor polynucleotide, wherein the donor polynucleotide may be inserted between the first site and the second site. The donor polynucleotide may be a donor exon comprising splice sites at the 5′ end and the 3′ end of the donor exon. The donor polynucleotide may comprise a wildtype sequence. The gene may be selected from NRL and NR2E3. The first guide RNA and/or the second guide RNA may target the Cas9 protein to a sequence comprising any one of SEQ ID NOS.: 1-4.

Disclosed herein are kits comprising a Cas nuclease or polynucleotide encoding the Cas nuclease, a first guide RNA and a second guide RNA, wherein the first guide RNA targets Cas9 cleavage of a first site 5′ of at least a first region of a gene and the second guide RNA targets Cas9 cleavage of a second site 3′ of the first region of the gene, thereby excising the region of the gene. The first guide RNA may target Cas9 cleavage of a first site 5′ of at least a first exon and the second guide RNA may target Cas9 cleavage of a second site 3′ of at least the first exon, thereby excising the at least first exon. The kit may comprise a donor polynucleotide, wherein the donor nucleic acid may be inserted between the first site and the second site. The donor polynucleotide may be a donor exon comprising splice sites at the 5′ end and the 3′ end of the donor exon. The donor polynucleotide may comprise a wildtype sequence. The gene may be selected from NRL and NR2E3. The first guide RNA and/or the second guide RNA may target the Cas9 protein to a sequence comprising any one of SEQ ID NOS.: 1-4.

Further disclosed herein are pharmaceutical compositions for treating a condition of an eye in a subject, comprising: a Cas nuclease or a polynucleotide encoding the Cas nuclease; and at least one guide RNA that is complementary to a portion of a gene selected from a NRL gene and a NR2E3 gene. The polynucleotide may encode the Cas protein and the at least one guide RNA are present in at least one viral vector. The polynucleotide encoding the Cas protein or the at least one guide RNA are present in a liposome. The at least one guide RNA may target the Cas protein to a sequence comprising any one of SEQ ID NOS.: 1-4. The pharmaceutical composition may be formulated as a liquid for administration with an eye dropper. The pharmaceutical composition may be formulated as a liquid for intravitreal administration.

Disclosed herein are methods of editing a gene in a cell comprising contacting the cell with a first guide RNA that hybridizes to a target site of a gene; a Cas nuclease that cleaves a strand of the gene at the target site; and a donor nucleic acid. The donor nucleic acid may be inserted into the gene via non-homologous end joining. The cell may be a post-mitotic cell. The gene may be a Mertk gene. The cell may be a cell in a retina of an eye of a subject.

Further disclosed herein are methods of treating retinal degeneration in a subject comprising contacting a retina of a subject with: a first guide RNA that hybridizes to a target site of a gene; a Cas nuclease that cleaves a strand of the gene at the target site; and a donor nucleic acid, wherein the donor nucleic acid is inserted into the gene via non-homologous end joining. The retinal degeneration may be retinitis pigmentosa. The gene may be a Mertk gene.

Disclosed herein are methods of treating beta thalassemia in a subject comprising contacting a hematopoietic stem/progenitor cell of a subject with: a first guide RNA that hybridizes to a target site of a hemoglobin gene; a Cas nuclease that cleaves a strand of the hemoglobin gene at the target site; and a donor nucleic acid wherein the donor nucleic acid is inserted into the gene via non-homologous end joining. The donor nucleic acid may replace a portion of the hemoglobin gene comprising a CD41/42 mutation.

Disclosed herein are methods of treating cancer in a subject comprising contacting a T cell of a subject with: a first guide RNA that hybridizes to a target site of a gene encoding an immune checkpoint inhibitor; and a Cas nuclease that cleaves a strand of the gene at the target site. The method may comprise contacting the T cell with a donor nucleic acid, wherein the donor nucleic acid is inserted into the gene via non-homologous end joining. The gene may be PDCD1 which encodes programmed cell death protein 1 (PD-1). The cancer may be a metastatic cancer. The cancer may be metastatic ovarian cancer, metastatic melanoma, metastatic non-small-cell lung cancer or metastatic renal cell carcinoma.

Further disclosed herein are methods of treating cancer in a subject comprising contacting a cancer cell of a subject with: a first guide RNA that hybridizes to a target site of a gene encoding an immune checkpoint inhibitor ligand; and a Cas nuclease that cleaves a strand of the gene at the target site. The gene may be CD274, also known as PDCD1LG1, which encodes programed-death ligand 1 (PD-L1). The gene may be PDCD1LG2 or programed-death ligand 2 (PD-L2). The methods may comprise contacting the tumor cell with a donor nucleic acid, wherein the donor nucleic acid is inserted into the gene via non-homologous end joining. The cancer may be a metastatic cancer. The cancer may be metastatic ovarian cancer, metastatic melanoma, metastatic non-small-cell lung cancer or metastatic renal cell carcinoma.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows adeno-associated virus (AAV) vectors, top vector encoding two guide RNAs for targeting an NRL gene, middle vector encoding two guide RNAs for targeting an NR2E3 gene, and a bottom vector encoding Cas9.

FIG. 1B shows targeting an NRL gene with two guide RNAs (6^{th} lane from the left) is more efficient than targeting the NRL gene with a single guide RNA (5^{th} lane from the left) in a T7E1 assay.

FIG. 1C shows targeting an NR2E3 gene with two guide RNAs (6^{th} lane from the left) is more efficient than targeting the NRL gene with a single guide RNA (5^{th} lane from the left) in a T7E1 assay.

FIG. 2 shows a representative schematic diagram of administering and assessing viral-mediated delivery of Cas9 and guide RNAs to treat retinitis pigmentosa (RP).

FIG. 3A shows staining of nuclei (DAPI), cone cells (mCAR), and viral expression (mCherry) in retinas of mice treated with viruses producing Cas9 and Nrl guide RNAs (top panels) versus control virus (bottom rows).

FIG. 3B shows a magnified view (relative to FIG. 3A) of staining of cone cells (mCAR).

FIG. 3C shows a magnified view (relative to FIG. 3A) of staining of cone cells (M-Opsin).

FIG. 3D shows a quantification of mCAR-positive cells in the lower outer nuclear layer (ONL) of the retina of mice treated with viruses producing Cas9 and Nrl guide RNAs versus mice treated with control virus.

FIG. 3E shows a quantification of mCAR-positive cells in the retina of mice treated with viruses producing Cas9 and Nrl guide RNAs versus mice treated with control virus, counting all mCAR-positive cones, which include previously existing cones plus newly reprogrammed cones.

FIG. 4 shows a quantification of outer nuclear layer (ONL) thickness in wildtype mice, mice with RP treated with control virus, and mice with RP that were treated with viruses producing Cas 9 and either Nrl guide RNAs or NR2E3 guide RNAs.

FIG. 5A shows improved vision, via electroretinography (ERG), in mice treated for RP with Cas9/gRNA (top panel) over similar mice treated with control virus (bottom panel).

FIG. 5B shows quantification of photopic ERG b wave amplitude in uninjected mice, AAV-gRNA injected mice, and AAV-Cas9, plus AAV-gRNA injected mice.

FIG. 6A shows luciferase assay for CD41/42-specific gRNA selection.

FIG. 6B shows comparison of Cas9 mRNA and Cas9RNP mediated HBB editing (left), screen of different ssODNs using Cas9 RNP-2 (right).

FIG. 6C shows droplet digital PCR analysis of HDR-mediated editing using ssODN(111/37).

FIG. 7A shows a schematic representation of the Mertk gene in both wild type and RCS rats. Pentagon, Cas9/gRNA target sequence. Black line within pentagon, Cas9 cleavage site.

FIG. 7B shows a schematic of Mertk gene correction AAV vectors. Exon 2 including surrounding intron is sandwiched by Cas9/gRNA target sequence and integrates within intron 1 of Mertk by HITI. The AAVs were packaged with serotype 8. Black half-arrows indicate PCR primer pairs to validate correct knock-in.

FIG. 7C shows a schematic of experimental design for Mertk gene correction in RCS rats. AAV-Cas9 and either AAV-rMertk-HITI or AAV AAV-rMertk-HDR were locally delivered to RCS rats by sub-retinal injection at 3 weeks and analyzed at 7-8 weeks.

FIG. 7D shows validation of correct gene knock-in in AAV-Cas9 and AAV-rMertk-HITI injected eyes by PCR.

FIG. 7E shows relative Mertk mRNA expression in an AAV-injected eye by RT-PCR. Number of animals for all bar graphs: RCS rats n=8, normal rats n=8, AAV-Cas9+AAV-rMertk-HITI treated group n=6, and AAV-Cas9+AAV-rMertk-HDR treated n=3.

FIG. 7F shows retinal morphology showing photoreceptor rescue in AAV-injected eyes. Increased preservation of photoreceptor outer nuclear layer (ONL) was observed compared to untreated and AAV-HDR treated RCS eyes which had only a very thin ONL (see brackets). Scale bars, 20 μm.

FIG. 7G shows improved Rod and cone mix response (left, wave forms; right, quantification bars), demonstrating improved b-wave value in AAV-Cas9 and AAV-rMertk-HITI injected eyes. Number of animals for all bar graphs: RCS rats n=8, normal rats n=8, AAV-Cas9+AAV-rMertk-HITI treated group n=8, and AAV-Cas9+AAV-rMertk-HDR treated n=6.

FIG. 711 shows improved 10 Hz flicker cone response in AAV-Cas9 and AAV-rMertk-HITI injected eyes. Number of animals for all bar graphs: RCS rats n=8, normal rats n=8, AAV-Cas9+AAV-rMertk-HITI treated group n=8, and AAV-Cas9+AAV-rMertk-HDR treated n=6. *P<0.05, Student's t-test.

FIG. 8 shows a schematic representation of Cas9-mediated restoration of a functional exon 2 to the Mertk gene.

FIG. 9 shows a schematic representation of AAV vector construction for split Cas9 Nrl genome editing.

FIG. 10A lists target sequences for Nrl knockdown and repression. PAM sequences are underlined.

FIG. 10B T7E1 assay of Nrl gRNAs in mouse embryonic fibroblasts. Figure discloses SEQ ID NOS 1-2 and 18-19, respectively, in order of appearance.

FIG. 11 shows a schematic representation of AAV construction for split KRAB-dCas9 Nrl gene repression.

FIGS. 12A-E demonstrates rod to cone cellular reprogramming in wild-type mice mediated by CRISPR/Cas9 knockdown or repression strategy using immunofluorescent analysis of cells in normal mouse retinas treated with AAV-Nrl gRNAs/split Cas9 or AAV-Nrl gRNAs/split Cas9. Rhodopsin, green; DAPI, blue. FIG. 12A shows experimental design for editing or repression of NRL in wild-type mice. Mice were treated at P7 and analyzed at P30. FIG. 12B shows analysis of mCAR⁺ cells (stained red). FIG. 12C shows analysis of M-Opsin⁺ cells (stained red). FIG. 12D shows quantification of total mCAR⁺ and M-Opsin⁺ cells. Results are shows as mean±s.e.m. (*p, 0.05, student's t-test). FIG. 12E shows RT-qPCR analysis of rod and cone-specific markers in treated wild-type retinas. RNA from each group was extracted from whole retina tissue. Results are shows as mean±s.e.m. (*p, 0.05, student's t-test).

FIGS. 12F-H demonstrates rod to cone cellular reprogramming in NRL-GFP mice mediated by CRISPR/Cas9 knockdown and repression strategy using AAV-Nrl gRNAs/split Cas9 or AAV-Nrl gRNAs/split Cas9. FIG. 12F shows experimental design for editing or repression of NRL in NRL-GFP mice. Mice were treated at P7 and analyzed at P30. FIG. 12G shows immunofluorescent analysis of mCAR⁺ cells from mice treated at P7 and harvested at P30. GFP, green; mCAR, red; DAPI, blue. FIG. 12H shows quantification of mCAR⁺ cells. Results are shown as mean±s.e.m. (*p<0.05, student's t-test).

FIG. 12I shows anatomic location of mCAR⁺ cells in wild-type retina treated with Nrl gRNAs/split Cas9. Arrows indicate ectopically-located mCAR⁺ cells at lower ONL and upper INL. FIG. 12J shows immunofluorescent analysis of Calbindin⁺ and mCAR⁺ cells in wild-type mice treated with AAV-Nrl-gRNAs/split Cas9 or AAV-Nrl-gRNAs/split KRAB dCas9. Calbinden, green; mCAAR, red; DAPI, blue. Arrows indicate Calbindin⁺/mCAR⁺ cells.

FIGS. 13A-G demonstrates CRISPR/Cas9 based knockdown or repression strategy rescuing retinal function in retinal degeneration mice using AAV-Nrl gRNAs/split Cas9 or AAV-Nrl gRNAs/split Cas9. FIG. 13A shows experimental design for editing or expression of NRL rd 10 mice. Mice were treated at P7 and analyzed at P60. Rod degeneration starts around P18, followed by cone degeneration a few days later. No rod and minimal cone activity is detected by P60. FIG. 13B shows quantification of b-wave amplitude in injected and uninjected rd10 mice (n=3, results are shown as mean±s.e.m., *p<0.05, paired student's t-test) and visual acuity of injected and uninjected rd10 mice (n=3, results are shown as mean±s.e.m., *p<0.05, student's t-test). FIG. 13C shows representative ERG wave records showing improved cone response in eyes injected with AAV-Nrl gRNAs/split Cas9 or AAV-Nrl gRNAs/split Cas9. FIG. 13D shows immunofluorescent analysis of mCAR⁺ cells in treated retinas. Rhodopsin, green; mCAR, red; DAPI, blue. FIG. 13E shows quantification of mCAR⁺ cells (mean±s.e.m., *p<0.05, student's t-test) and ONL thickness (mean±s.e.m., *p<0.05) in treated retinas. FIG. 13F shows immunofluorescent analysis of M-Opsin⁺ cells in treated retinas. Rhodopsin, green; M-Opsin, red; DAPI, blue. FIG. 13G shows quantification of M-Opsin⁺ cells in treated retinas. Results are shown as mean±s.e.m. (*p<0.05, student's t-test).

FIGS. 14A-C exhibits CRISPR/CAS9 knockdown and repression strategy rebooting retinal function in 3-month old retinal degeneration mice using AAV-Nrl gRNAs/split Cas9 or AAV-Nrl gRNAs/split Cas9. Mice were treated at P90 and analyzed at P130. No rod or cone activity is detected by P90 in Rd10 mice. FIG. 14A shows experimental design for editing or repression of NRL in Rd10 mice. FIG. 14B shows immunofluorescent analysis of mCAR⁺ cells in treated retinas. Rhodopsin, green; mCAR, red; DAPI, blue. FIG. 14C shows quantification of mCAR⁺ cells (*p<0.05, student's t-test), ONL thickness (*p<0.05), b-wave amplitude (n=3, *p<0.05, paired student's t-test), and visual acuity (n=3, *p<0.05, student's t-test) in rd10 treated retinas. FIG. 14D shows immunofluorescent analysis of Calbindin⁺ and Opsin⁺ cells in treated adult retinal degeneration mice treated with AAV-Nrl gRNAs/split Cas9 or AAV-Nrl gRNAs/split Cas9, demonstrating horizontal cell to cone cell reprogramming in retinal degeneration mice. Rd10 mice were treated at 3-months and harvested 6 weeks later (P130). Calbindin, red; Opsin, green; DAPI, blue. Arrows indicate Calbindin⁺/Opsin⁺ cells.

FIGS. 15A-C exhibits CRISP/Cas9 knockdown and repression strategy rebooting retinal function in 3-month old FvB retinal degeneration mice using AAV-Nrl gRNAs/split Cas9 or AAV-Nrl gRNAs/split Cas9. Mice were treated at P90 and analyzed at P130. FIG. 15A shows experimental design for editing or repression of NRL in FvB mice. FIG. 15B shows immunofluorescent analysis of mCAR⁺ cells in treated retinas. Rhodopsin, green; mCAR, red; DAPI, blue. FIG. 15C shows quantification of mCAR⁺ cells (*p<0.05, student's t-test), ONL thickness (*p<0.05), b-wave amplitude (n=3, *p<0.05, paired student's t-test), and visual acuity (n=3, *p<0.05, student's t-test) in rd10 treated retinas. All results are shown as mean±s.e.m.

### DETAILED DESCRIPTION OF THE DISCLOSURE

Gene therapy shows great promise in treating many human diseases. However, one major drawback of the current technology is that it can only be directed to a particular mutation or a single gene at best, which makes gene therapy difficult to apply to a broad patient population. Similarly, repair and regeneration of tissues using endogenous or autologous stem cells represents an important goal in regenerative medicine. However, this approach is hindered by the requirement that the starting cells possess normal genetic makeup and function, which in many cases is not feasible as the autologous cell harbors the genetic mutation that the gene therapy aims to overcome. Provided herein are methods to overcome the above challenges with cellular reprogramming which switches a cell type that is sensitive to a mutation to a functionally related cell type that is resistant to the same mutation, therefore preserve the tissue and function. This approach is based on the premise that 1) a mutation usually causes its detrimental effect in only a particular cell type; 2) a combination of transcriptional factors enables determination of a cellular fate, and 3) there is developmental plasticity that allows for direct conversion in vivo between closely related, terminally differentiated mature cell types such as pancreas, cardiac and neural cells. Furthermore, distantly related cells can also be directly converted in vivo by appropriate combinations of developmentally relevant transcription factors.

Provided herein are methods utilizing a homology-independent targeted integration (HITI) strategy, based on clustered regularly interspaced short palindromic repeat-Cas9 (CRISPR-Cas9). These methods provide efficient targeted knock-in in both dividing and non-dividing cells. These methods may be performed in vitro and in vivo. These methods provide for on-target transgene insertion in post-mitotic cells, e.g., the brain, of postnatal mammals.

Retinitis pigmentosa RP is one of the most common degenerative diseases of the eye, affecting over one million patients worldwide. It can be caused by numerous mutations in over 200 genes. RP is characterized with primary rod photoreceptor death and degeneration, followed by secondary cone death. Acute gene knockout of rod determinant NRL reprograms adult rods into cone-like cells, rendering them resistant to effects of mutations in RP-specific genes on rod photoreceptors and consequently preventing secondary cone loss. NRL acts as a master switch gene between rods and cones and activates a key downstream transcriptional factor NR2E3. NRL and NR2E3 function in concert to activate a rod-specific gene transcription network and control rod differentiation and fate. Loss of function in either NRL or NR2E2 reprograms rods to a cone cell fate. This system provides an opportunity for proof of concept that therapies can be developed wherein cells are reprogrammed from those that are sensitive to a mutation to those that are resistant to the mutation.

Provided herein are methods for treatment of conditions comprising targeted inactivation of a gene harboring a mutation in a cell type that is sensitive (e.g., dysfunctional or deleterious to a subject with the cell) to the mutation. Provided herein are examples of these methods, including methods for treatment of RP and other retinal conditions using in vivo rod to cone reprogramming by targeted inactivation of NRL or NR2E3 in the retina using an adeno-associated virus (AAV)-delivery of CRISPR/Cas9 (see, e.g., Example 12). Examples demonstrate that a rod to cone specific cell fate can be reprogrammed by inactivation of a rod photoreceptor cell fate with consequent retinal photoreceptor reservation and visual function rescue. These results point to a novel treatment approach that is gene and mutation independent and may have broad implications for genetic disease therapy.

### Therapeutic Platforms

Provided herein are methods of treating a subject for a genetic condition comprising administering to a cell of a first cell type of the subject a therapeutic agent disclosed herein that modifies expression of a gene in the first cell, wherein the gene encodes a protein having a function specific to the first cell type. Modifying expression of the gene may result in reprogramming the cell from the first cell type to a second cell type. By way of non-limiting example, the genetic condition may be retinitis pigmentosa, the gene may be selected from NRL and NR2E3, and the therapeutic agent may be a virus encoding a Cas nuclease and guide RNA(s) targeting the gene. The method may comprise administering the therapeutic agent to a retinal cell, such as a rod photoreceptor cell, also referred to herein as a “rod.” The method may result in reprogramming rods to cones, rescuing retinal degeneration and restoring retinal functions. Thus the first cell type may be a rod and the second cell type is a cone, (see, e.g., Example 13). Although rod to cone reprogramming may lead to a loss of rod number and function with potential consequent night blindness, the subject may be willing to tolerate night blindness.

Provided herein are methods of re-programming a cell from a first cell type to a second cell type, comprising contacting the cell with a guide RNA that hybridizes to a target site of a gene, wherein the gene encodes a protein that contributes to a cell type specific function of the cell; and a Cas nuclease that cleaves a strand of the gene at the target site, wherein cleaving the strand modifies expression of the gene such that the cell can no longer perform the cell type specific function, thereby re-programming the cell to the second cell type.

The term “re-programming,” as used herein, refers to genetically altering at least one gene in a cell to switch the cell from a first cell type to a second cell type. The first cell type may be a more differentiated version of the second cell type or vice versa. The first cell type may be functionally related to the second cell type. For example, the first cell type and the second cell type may provide a function related to vision. Also by way of non-limiting example, the first cell type and the second cell type may provide a function related to brain activity, neuronal activity, muscle activity, immune activity, sensory activity, cardiovascular activity, cellular proliferation, cellular senescence, and cellular apoptosis. Genetically altering the gene may comprise silencing the gene, thereby inhibiting the production of protein(s) encoded by the gene. Silencing the gene may comprise introducing a nonsense mutation into the gene to produce a non-functional protein. The nonsense mutation may be introduced by using gene editing to create an artificial splice variant, wherein the artificial splice variant is missing at least one exon or portion thereof.

The term “cell type specific function,” as used herein, refers to a function specific to a cell type. In some cases the function is specific to a single cell type only. For example, the cell type specific function may be light vision and the single cell type is a cone photoreceptor cell. In some cases, the function is specific to a subset of cells. For example, the cell type specific function may be vision in general, and the subset of cells may be photoreceptor cells such as rods, cones, and photosensitive retinal ganglion cells.

The terms “first cell type” and “second cell type” are only used herein to distinguish one cell type from another in the context it is being immediately used. By no means should the methods or compositions disclosed herein be restricted by their order in one section of this application relative another section of this application.

A first cell type disclosed herein may be sensitive to a mutation. “Sensitive to the mutation” means that the mutation in a gene in that cell will result in a functional effect for that cell. A second cell type disclosed herein may be resistant to the mutation. “Resistant to the mutation” means that the mutation in a gene in that cell will not result in any functional effect for that cell, or that the mutation in a gene in that cell will result in a functional effect that is acceptable, not deleterious to a subject in which the cell is present, or a functional effect with little to no consequence for a subject in which the cell is present. For example, a cell type that is resistant to the mutation may be a cell type that does not express the gene or expresses a negligible amount of the gene. The cell type that is resistant to the mutation may be a cell type that expresses the gene, but the functional role of the gene in that cell type is not affected by the mutation. The cell type that is sensitive to the mutation performs a cell-type specific function, wherein the cell-type specific function is regulated or controlled by expression of the gene that can harbor the mutation. When the mutation occurs in the gene, the cell-type specific function is lost or altered. The methods disclosed herein comprise editing the gene, resulting in re-programming the first cell type (sensitive to the mutation) to the second cell type (resistant to the mutation).

Provided herein are methods of treating retinal degeneration. Retinal degeneration encompasses a number of diseases, such as retinitis pigmentosa, macular degeneration and glaucoma. The methods may comprise re-programming a retinal cell from a rod photoreceptor cell type to a cone photoreceptor cell type, comprising contacting the retinal cell with a guide RNA that hybridizes to a target site of a gene disclosed herein, wherein the gene encodes a protein that contributes to night or color vision function of the cell; and a Cas nuclease that cleaves a strand of the gene at the target site, wherein cleaving the strand modifies expression of the gene such that the retinal cell can no longer perform night or color vision function, thereby re-programming the retinal cell to the cone photoreceptor cell type. The cone photoreceptor cell type may be capable of providing light vision to a subject. The gene may be selected from NRL, NR2E3, GNAT1, ROR beta, OTX2, CRX and THRB. The gene may be NRL. The gene may be NR2E3.

Provided herein are methods of treating retinal degeneration. Retinal degeneration encompasses a number of diseases, such as retinitis pigmentosa, macular degeneration and glaucoma. The methods may comprise re-programming a retinal cell from a first cell type to a second cell type. The first cell type may be a rod. The first cell type may be a cell other than a rod or cone. The first cell type may be a neuron. The first cell type may be an interneuron. The first cell type may be a neuronal stem cell or a neuronal precursor cell (a multipotent or pluripotent cell with the capability to differentiate into a neuronal cell). An advantage of using cells such as interneurons or cell other than rods, is that these methods can be used to provide sight to end stage RP patients who have completely lost both rod and cone receptors. The second cell type may be a cone. The second cell type may be an intermediate cell. The intermediate cell may be a cell that has been subjected to re-programming as described herein (e.g., treated with a Cas nuclease and guide RNA or RNAi). The intermediate cell may be a rod cell, in which rod cell gene expression has been down regulated. Down-regulation of rod cell gene expression may decrease the effects of rod-specific mutations. “Rod-specific mutations” as used herein generally refers to mutations in genes that affect rod cell function and phenotype. In other words, rod cells may be sensitive to rod-cell mutations. Such cells could provide tissue structural support to maintain normal architecture and function. These cells may also secrete trophic factors crucial to maintaining growth and survival of endogenous cone cells.

The methods may comprise re-programming a retinal cell from a rod photoreceptor cell type to a pluripotent cell type, comprising contacting the retinal cell with a guide RNA that hybridizes to a target site of a gene disclosed herein, wherein the gene encodes a protein that contributes to night or color vision function of the cell; and a Cas nuclease that cleaves a strand of the gene at the target site, wherein cleaving the strand modifies expression of the gene such that the retinal cell can no longer perform night or color vision function, thereby re-programming the retinal cell to the pluripotent cell type. The pluripotent cell type may be a multi-potent retinal progenitor cell, meaning a cell that has the potential to develop into a rod or cone when placed in the retina and/or subjected to environmental stimuli of the retina. The pluripotent cell type may be a cell type that is intermediate to a cone and a rod. The cell type that is intermediate to the cone and the rod may be a retinal ganglion pluripotent cell. In the normal retinal developmental process, the retinal ganglion pluripotent cell will differentiate into a cone or rod. The gene may be selected from NRL, NR2E3, GNAT1, ROR beta, OTX2, CRX and THRB. The gene may be NRL. The gene may be NR2E3.

Provided herein are methods of treating cancer. By way of non-limiting example, cancer may include colon cancer, B cell lymphoma, glioblastoma, retinoblastoma, and breast cancer. The methods may comprise re-programming a cancer cell from a malignant cell type to a benign cell type, comprising contacting the cancer cell with a guide RNA that hybridizes to a target site of a gene disclosed herein, wherein the gene encodes a protein that contributes to proliferation of the cell; and a Cas nuclease that cleaves a strand of the gene at the target site, wherein cleaving the strand modifies expression of the gene such that the cancer cell can no longer aberrantly proliferate, thereby re-programming the cancer cell to the benign cell type. By way of non-limiting example, the first cell type may be a colon cancer cell, the second cell type may be a benign intestinal cell or benign colon cell, and the gene may be selected from APC, MYH1, MYH2, MYH3, MLH1, MSH2, MSH6, PMS2, EPCAM, POLE1, POLD1, NTHL1, BMPR1A, SMAD4, PTEN, and STK11. Also, by way of non-limiting example, the first cell type may be a malignant B cell, the second cell type may be a benign macrophage, and the gene may be PU.1, CD19, CD20, CD34, CD38, CD45 or CD78. The first cell type may be a malignant B cell, the second cell type may be a benign macrophage, and the gene may be C-MYC, CCND1, BCL2, BCL6, TP53, CDKN2A, CREBBP or EP300. The second cell type may express higher RNA/protein levels of CD68, CD11b, F480, Cd11c, or Ly6g than the first cell type. Also by way of non-limiting example, the first cell type may be an estrogen receptor positive and/or Her2 positive breast cancer cell, the second cell type may be an estrogen receptor negative and/or estrogen receptor negative breast cancer cell, and the gene may be selected from an estrogen receptor gene, a Her2 gene, and a combination thereof.

The methods of treating cancer disclosed herein may comprise modifying the gene such that the cancer cell loses an ability to metastasize. The method may comprise modifying the gene such that the cancer cell loses an ability to promote tumor vascularization.

### RNA Interference (RNAi)

Provided herein are methods of administering an anti-sense oligonucleotide capable of inhibiting expression of a gene in a cell via RNA interference. Inhibiting the gene may result in converting the cell from a first cell type to a second cell type. The first cell type or cell type may be any cell type disclosed herein. In some embodiments, the anti-sense oligonucleotide comprises a modification providing resistance to digestion or degradation by naturally-occurring DNase enzymes. In some embodiments, the modification is a modification of the anti-sense oligonucleotide's phosphodiester backbone using a solid-phase phosphoramidite method during its synthesis. This will effectively render most forms of DNase ineffective to the anti-sense oligonucleotide.

In some embodiments, the anti-sense oligonucleotide comprises a delivery system that facilitates or enhances uptake of the anti-sense oligonucleotide most efficiently in two methods. In some embodiments, the delivery system comprises a liposome or lipid container that is easily taken in by a human cell. In some embodiments, the delivery system is a system that is mediated by the tat protein, which allows easy transfer of large molecules, like oligonucleotides, through the cell membrane.

In some embodiments, the anti-sense oligonucleotide is a small hairpin RNA (shRNA). These strands of RNA silence the gene by targeting the mRNA produced by the gene of interest. In some embodiments, the shRNA may be custom-designed via computer software and manufactured commercially using a design template. In some embodiments, the shRNA is delivered using bacterial plasmids, circular strands of bacterial DNA, or viruses carrying viral vectors.

In some embodiments, the anti-sense oligonucleotide targets a RNA encoded by a NR2E3 gene. In some embodiments, the anti-sense oligonucleotide targets a RNA encoded by a NRL gene. In some embodiments, the anti-sense oligonucleotide targets a RNA encoded by a gene encoding an opsin protein. In some embodiments, the anti-sense oligonucleotide targets a RNA encoded by a rhodopsin gene.

In some embodiments, the siRNA is between about 18 nucleotides and about 30 nucleotides in length. In some embodiments, the siRNA is 18 nucleotides in length. In some embodiments, the siRNA is 19 nucleotides in length. In some embodiments, the siRNA is 20 nucleotides in length. In some embodiments, the siRNA is 21 nucleotides in length. In some embodiments, the siRNA is 22 nucleotides in length. In some embodiments, the siRNA is 23 nucleotides in length. In some embodiments, the siRNA is 24 nucleotides in length. In some embodiments, the siRNA is 25 nucleotides in length.

### Gene Editing

Provided herein are methods for gene editing a gene in a cell, wherein the gene editing results in converting the cell from a first cell type to a second cell type. By way of non-limiting example, the methods may be used for the treatment of a retinal condition. Further provided herein is a cell, wherein a gene in the cell is modified by a method disclosed herein. By way of non-limiting example, the cell is a cell of the retina, also referred to as a retinal cell. In some embodiments, methods and cells disclosed herein utilize genome editing to modify a target gene in a cell, for the treatment of the retinal condition. In some embodiments, methods and cells disclosed herein utilize a nuclease or nuclease system. In some embodiments, nuclease systems comprise site-directed nucleases. Suitable nucleases include, but are not limited to, CRISPR-associated (Cas) proteins or Cas nucleases including type I CRISPR-associated (Cas) polypeptides, type II CRISPR-associated (Cas) polypeptides, type III CRISPR-associated (Cas) polypeptides, type IV CRISPR-associated (Cas) polypeptides, type V CRISPR-associated (Cas) polypeptides, and type VI CRISPR-associated (Cas) polypeptides; zinc finger nucleases (ZFN); transcription activator-like effector nucleases (TALEN); meganucleases; RNA-binding proteins (RBP); CRISPR-associated RNA binding proteins; recombinases; flippases; transposases; Argonaute proteins; any derivative thereof; any variant thereof; and any fragment thereof. In some embodiments, site-directed nucleases disclosed herein can be modified in order to generate catalytically dead nucleases that are able to site-specifically bind target sequences without cutting, thereby blocking transcription and reducing target gene expression.

In some embodiments, methods and cells disclosed herein utilize a nucleic acid-guided nuclease system. In some embodiments, methods and cells disclosed herein utilize a clustered regularly interspaced short palindromic repeats (CRISPR), CRISPR-associated (Cas) protein system for modification of a nucleic acid molecule. In some embodiments, the CRISPR/Cas systems disclosed herein comprise a Cas nuclease and a guide RNA. In some embodiments, the CRISPR/Cas systems disclosed herein comprise a Cas nuclease, a guide RNA, and a repair template. The guide RNA directs the Cas nuclease to a target sequence, where the Cas nuclease cleaves or nicks the target sequence, thereby creating a cleavage site. In some embodiments, the Cas nuclease generates a double stranded break (DSB) that is repaired via nonhomology end joining (NHEJ). However, in some embodiments, unmediated or non-directed NHEJ-mediated DSB repair results in disruption of an open reading frame that leads to undesirable consequences. To circumvent these issues, in some embodiments, the methods disclosed herein comprise the use of a repair template to be inserted at the cleavage site, allowing for control of the final edited gene sequence. This use of a repair template may be referred to as homology directed repair (HDR). In some embodiments, methods and cells disclosed herein utilize homology-independent targeted integration (HITI). HITI may allow for efficient targeted knock-in in both dividing and non-dividing cells in vitro, and more importantly, for in vivo on-target transgene insertion in post-mitotic cells, e.g., the brain, of postnatal mammals.

In some embodiments, the repair template comprises a wildtype sequence corresponding to the target gene. In some embodiments, the repair template comprises a desired sequence to be delivered to the cleavage site. In some embodiments, the desired sequence is not the wildtype sequence. In some embodiments, the desired sequence is identical to the target sequence with the exception of one or more edited nucleotides to correct or alter the expression/activity of the target gene. For example, the desired sequence may comprise a single nucleotide difference as compared to the target sequence that contained a single nucleotide polymorphism, wherein the single nucleotide difference is a substitution for the nucleotide of the single nucleotide polymorphism that restores wildtype expression/activity or altered expression/activity to the target gene.

Any suitable CRISPR/Cas system may be used for the methods and compositions disclosed herein. The CRISPR/Cas system may be referred to using a variety of naming systems. Exemplary naming systems are provided in Makarova, K. S. et al, “An updated evolutionary classification of CRISPR-Cas systems,” Nat Rev Microbiol (2015) 13:722-736 and Shmakov, S. et al, “Discovery and Functional Characterization of Diverse Class 2 CRISPR-Cas Systems,” Mol Cell (2015) 60:1-13. The CRISPR/Cas system may be a type I, a type II, a type III, a type IV, a type V, a type VI system, or any other suitable CRISPR/Cas system. The CRISPR/Cas system as used herein may be a Class 1, Class 2, or any other suitably classified CRISPR/Cas system. The Class 1 CRISPR/Cas system may use a complex of multiple Cas proteins to effect regulation. The Class 1 CRISPR/Cas system may comprise, for example, type I (e.g., I, IA, IB, IC, ID, IE, IF, IU), type III (e.g., III, IIIA, IIIB, IIIC, IIID), and type IV (e.g., IV, IVA, IVB) CRISPR/Cas type. The Class 2 CRISPR/Cas system may use a single large Cas protein to effect regulation. The Class 2 CRISPR/Cas systems may comprise, for example, type II (e.g., II, IIA, IIB) and type V CRISPR/Cas type. CRISPR systems may be complementary to each other, and/or can lend functional units in trans to facilitate CRISPR locus targeting.

The Cas protein may be a type I, type II, type III, type IV, type V, or type VI Cas protein. The Cas protein may comprise one or more domains. Non-limiting examples of domains include, a guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. The guide nucleic acid recognition and/or binding domain may interact with a guide nucleic acid. The nuclease domain may comprise catalytic activity for nucleic acid cleavage. The nuclease domain may lack catalytic activity to prevent nucleic acid cleavage. The Cas protein may be a chimeric Cas protein that is fused to other proteins or polypeptides. The Cas protein may be a chimera of various Cas proteins, for example, comprising domains from different Cas proteins.

Non-limiting examples of Cas proteins include c2c1, C2c2, c2c3, Cas1, Cas1B, Cas2, Cas3, Cas4, Cas5, Cas5e (CasD), Cash, Cas6e, Cas6f, Cas7, Cas8a, Cas8a1, Cas8a2, Cas8b, Cas8c, Cas9 (Csn1 or Csx12), Cas10, Cas10d, Cas1O, Cas1Od, CasF, CasG, CasH, Cpf1, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, and Cul966, and homologs or modified versions thereof.

The Cas protein may be from any suitable organism. Non-limiting examples include Streptococcus pyogenes, Streptococcus thermophilus, Streptococcus sp., Staphylococcus aureus, Nocardiopsis dassonvillei, Streptomyces pristinae spiralis, Streptomyces viridochromo genes, Streptomyces viridochromogenes, Streptosporangium roseum, Streptosporangium roseum, AlicyclobacHlus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Pseudomonas aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Leptotrichia shahii, and Francisella novicida. In some aspects, the organism is Streptococcus pyogenes (S. pyogenes). In some aspects, the organism is Staphylococcus aureus (S. aureus). In some aspects, the organism is Streptococcus thermophilus (S. thermophilus).

The Cas protein may be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida. The term, “derived,” in this instance, is defined as modified from the naturally-occurring variety of bacterial species to maintain a significant portion or significant homology to the naturally-occurring variety of bacterial species. A significant portion may be at least 10 consecutive nucleotides, at least 20 consecutive nucleotides, at least 30 consecutive nucleotides, at least 40 consecutive nucleotides, at least 50 consecutive nucleotides, at least 60 consecutive nucleotides, at least 70 consecutive nucleotides, at least 80 consecutive nucleotides, at least 90 consecutive nucleotides or at least 100 consecutive nucleotides. Significant homology may be at least 50% homologous, at last 60% homologous, at least 70% homologous, at least 80% homologous, at least 90% homologous, or at least 95% homologous. The derived species may be modified while retaining an activity of the naturally-occurring variety.

In some embodiments, the CRISPR/Cas systems utilized by the methods and cells described herein are Type-II CRISPR systems. In some embodiments, the Type-II CRISPR system comprises a repair template to modify the nucleic acid molecule. The Type-II CRISPR system has been described in the bacterium Streptococcus pyogenes, in which Cas9 and two non-coding small RNAs (pre-crRNA and tracrRNA (trans-activating CRISPR RNA)) act in concert to target and degrade a nucleic acid molecule of interest in a sequence-specific manner (see Jinek et al., “A Programmable Dual-RNA-Guided DNA Endonuclease in Adaptive Bacterial Immunity,” Science 337(6096):816-821 (August 2012, epub Jun. 28, 2012)). In some embodiments, the two non-coding small RNAs are connected to create a single nucleic acid molecule, referred to as the guide RNA.

In some embodiments, methods and cells disclosed herein use a guide nucleic acid. The guide nucleic acid refers to a nucleic acid that can hybridize to another nucleic acid. The guide nucleic acid may be RNA. The guide nucleic acid may be DNA. The guide nucleic acid that is DNA may be more stable than a guide RNA. The guide nucleic acid may be programmed to bind to a sequence of nucleic acid site-specifically. The nucleic acid to be targeted, or the target nucleic acid, may comprise nucleotides. The guide nucleic acid may comprise nucleotides. A portion of the target nucleic acid may be complementary to a portion of the guide nucleic acid. The guide nucleic acid may comprise a polynucleotide chain and can be called a “single guide nucleic acid” (i.e. a “single guide nucleic acid”). The guide nucleic acid may comprise two polynucleotide chains and may be called a “double guide nucleic acid” (i.e. a “double guide nucleic acid”). If not otherwise specified, the term “guide nucleic acid” is inclusive, referring to both single guide nucleic acids and double guide nucleic acids.

The guide nucleic acid can comprise a segment that can be referred to as a “guide segment” or a “guide sequence.” The guide nucleic acid may comprise a segment that can be referred to as a “protein binding segment” or “protein binding sequence.”

The guide nucleic acid may comprise one or more modifications (e.g., a base modification, a backbone modification), to provide the nucleic acid with a new or enhanced feature (e.g., improved stability). The guide nucleic acid may comprise a nucleic acid affinity tag. The guide nucleic acid may comprise a nucleoside. The nucleoside may be a base-sugar combination. The base portion of the nucleoside may be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group may be linked to the 2′, the 3′, or the 5′ hydroxyl moiety of the sugar. In forming guide nucleic acids, the phosphate groups may covalently link adjacent nucleosides to one another to form a linear polymeric compound. In turn, the respective ends of this linear polymeric compound may be further joined to form a circular compound; however, linear compounds are generally suitable. In addition, linear compounds may have internal nucleotide base complementarity and may therefore fold in a manner as to produce a fully or partially double-stranded compound. Within guide nucleic acids, the phosphate groups arecommonly referred to as forming the internucleoside backbone of the guide nucleic acid. The linkage or backbone of the guide nucleic acid may be a 3′ to 5′ phosphodiester linkage.

The guide nucleic acid may comprise a modified backbone and/or modified internucleoside linkages. Modified backbones may include those that retain a phosphorus atom in the backbone and those that do not have a phosphorus atom in the backbone.

Suitable modified guide nucleic acid backbones containing a phosphorus atom therein may include, for example, phosphorothioates, chiral phosphorothioates, phosphorodithioates, phosphotriesters, aminoalkylphosphotriesters, methyl and other alkyl phosphonates such as 3′-alkylene phosphonates, 5′-alkylene phosphonates, chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage. Suitable guide nucleic acids having inverted polarity can comprise a single 3′ to 3′ linkage at the 3′-most internucleotide linkage (i.e. a single inverted nucleoside residue in which the nucleobase is missing or has a hydroxyl group in place thereof). Various salts (e.g., potassium chloride or sodium chloride), mixed salts, and free acid forms can also be included.

The guide nucleic acid may comprise one or more phosphorothioate and/or heteroatom internucleoside linkages, in particular —CH2-NH—O—CH2-, —CH2-N(CH3)-O—CH2- (i.e. a methylene (methylimino) or MMI backbone), —CH2-O—N(CH3)-CH2-, —CH2-N(CH3)-N(CH3)-CH2- and —O—N(CH3)-CH2-CH2- (wherein the native phosphodiester internucleotide linkage is represented as —O—P(═O)(OH)—O—CH2-).

The guide nucleic acid may comprise a morpholino backbone structure. For example, the guide nucleic acid may comprise a 6-membered morpholino ring in place of a ribose ring. In some of these embodiments, a phosphorodiamidate or other non-phosphodiester internucleoside linkage replaces a phosphodiester linkage.

The guide nucleic acid may comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These may include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH2 component parts.

The guide nucleic acid may comprise a nucleic acid mimetic. The term “mimetic” is intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety may be maintained for hybridization with an appropriate target nucleic acid. One such nucleic acid may be a peptide nucleic acid (PNA). In a PNA, the sugar-backbone of a polynucleotide may be replaced with an amide containing backbone, in particular an aminoethylglycine backbone. The nucleotides may be retained and are bound directly or indirectly to aza nitrogen atoms of the amide portion of the backbone. The backbone in PNA compounds may comprise two or more linked aminoethylglycine units which gives PNA an amide containing backbone. The heterocyclic base moieties may be bound directly or indirectly to aza nitrogen atoms of the amide portion of the backbone.

The guide nucleic acid may comprise linked morpholino units (i.e. morpholino nucleic acid) having heterocyclic bases attached to the morpholino ring. Linking groups c may an link the morpholino monomeric units in a morpholino nucleic acid. Non-ionic morpholino-based oligomeric compounds may have less undesired interactions with cellular proteins. Morpholino-based polynucleotides may be nonionic mimics of guide nucleic acids. A variety of compounds within the morpholino class may be joined using different linking groups. A further class of polynucleotide mimetic may be referred to as cyclohexenyl nucleic acids (CeNA). The furanose ring normally present in a nucleic acid molecule may be replaced with a cyclohexenyl ring. CeNA DMT protected phosphoramidite monomers may be prepared and used for oligomeric compound synthesis using phosphoramidite chemistry. The incorporation of CeNA monomers into a nucleic acid chain may increase the stability of a DNA/RNA hybrid. CeNA oligoadenylates may form complexes with nucleic acid complements with similar stability to the native complexes. A further modification may include Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage may be a methylene (—CH2-), group bridging the 2′ oxygen atom and the 4′ carbon atom wherein n is 1 or 2. LNA and LNA analogs may display very high duplex thermal stabilities with complementary nucleic acid (Tm=+3 to +10° C.), stability towards 3′-exonucleolytic degradation and good solubility properties.

The guide nucleic acid may comprise one or more substituted sugar moieties. Suitable polynucleotides can comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. Particularly suitable are O((CH2)nO) mCH3, O(CH2)nOCH3, O(CH2)nNH2, O(CH2)nCH3, O(CH2)nONH2, and O(CH2)nON((CH2)nCH3)2, where n and m are from 1 to about 10. The sugar substituent group may be selected from: C1 to C10 lower alkyl, substituted lower alkyl, alkenyl, alkynyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an guide nucleic acid, or a group for improving the pharmacodynamic properties of an guide nucleic acid, and other substituents having similar properties. A suitable modification can include 2′-methoxyethoxy (2′-O—CH2 CH2OCH3, also known as 2′-O-(2-methoxyethyl) or 2′-MOE i.e., an alkoxyalkoxy group). A further suitable modification may include 2′-dimethylaminooxyethoxy, (i.e., a O(CH2)2ON(CH3)2 group, also known as 2′-DMAOE), and 2′-dimethylaminoethoxyethoxy (also known as 2′-O-dimethyl-amino-ethoxy-ethyl or 2′-DMAEOE), i.e., 2′-O—CH2-O—CH2-N(CH3)2.

Other suitable sugar substituent groups may include methoxy (—O—CH3), aminopropoxy (—OCH2CH2CH2NH2), allyl (—CH2-CH═CH2), —O-allyl (—O—CH2-CH═CH2) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked nucleotides and the 5′ position of 5′ terminal nucleotide. Oligomeric compounds may also have sugar mimetics such as cyclobutyl moieties in place of the pentofuranosyl sugar.

The guide nucleic acid may also include nucleobase (often referred to simply as “base”) modifications or substitutions. As used herein, “unmodified” or “natural” nucleobases can include the purine bases, (e.g. adenine (A) and guanine (G)), and the pyrimidine bases, (e.g. thymine (T), cytosine (C) and uracil (U)). Modified nucleobases may include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (Hpyrido(3′,2′:4,5)pyrrolo(2,3-d)pyrimidin-2-one).

Heterocyclic base moieties may include those in which the purine or pyrimidine base is replaced with other heterocycles, for example 7-deaza-adenine, 7-deazaguanosine, 2-aminopyridine and 2-pyridone. Nucleobases may be useful for increasing the binding affinity of a polynucleotide compound. These may include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions can increase nucleic acid duplex stability by 0.6-1.2° C. and can be suitable base substitutions (e.g., when combined with 2′-O-methoxyethyl sugar modifications).

A modification of a guide nucleic acid may comprise chemically linking to the guide nucleic acid one or more moieties or conjugates that can enhance the activity, cellular distribution or cellular uptake of the guide nucleic acid. These moieties or conjugates may include conjugate groups covalently bound to functional groups such as primary or secondary hydroxyl groups. Conjugate groups may include, but are not limited to, intercalators, reporter molecules, polyamines, polyamides, polyethylene glycols, polyethers, groups that enhance the pharmacodynamic properties of oligomers, and groups that can enhance the pharmacokinetic properties of oligomers. Conjugate groups may include, but are not limited to, cholesterols, lipids, phospholipids, biotin, phenazine, folate, phenanthridine, anthraquinone, acridine, fluoresceins, rhodamines, coumarins, and dyes. Groups that enhance the pharmacodynamic properties include groups that improve uptake, enhance resistance to degradation, and/or strengthen sequence-specific hybridization with the target nucleic acid. Groups that can enhance the pharmacokinetic properties include groups that improve uptake, distribution, metabolism or excretion of a nucleic acid. Conjugate moieties may include but are not limited to lipid moieties such as a cholesterol moiety, cholic acid a thioether, (e.g., hexyl-S-tritylthiol), a thiocholesterol, an aliphatic chain (e.g., dodecandiol or undecyl residues), a phospholipid (e.g., di-hexadecyl-rac-glycerol or triethylammonium 1,2-di-O-hexadecyl-rac-glycero-3-H-phosphonate), a polyamine or a polyethylene glycol chain, or adamantane acetic acid, a palmityl moiety, or an octadecylamine or hexylamino-carbonyl-oxycholesterol moiety.

A modification may include a “Protein Transduction Domain” or PTD (i.e. a cell penetrating peptide (CPP)). The PTD may refer to a polypeptide, polynucleotide, carbohydrate, or organic or inorganic compound that facilitates traversing a lipid bilayer, micelle, cell membrane, organelle membrane, or vesicle membrane. The PTD may be attached to another molecule, which can range from a small polar molecule to a large macromolecule and/or a nanoparticle, and can facilitate the molecule traversing a membrane, for example going from extracellular space to intracellular space, or cytosol to within an organelle. The PTD may be covalently linked to the amino terminus of a polypeptide. The PTD may be covalently linked to the carboxyl terminus of a polypeptide. The PTD may be covalently linked to a nucleic acid. Exemplary PTDs may include, but are not limited to, a minimal peptide protein transduction domain; a polyarginine sequence comprising a number of arginines sufficient to direct entry into a cell (e.g., 3, 4, 5, 6, 7, 8, 9, 10, or 10-50 arginines), a VP22 domain, a Drosophila Antennapedia protein transduction domain, a truncated human calcitonin peptide, polylysine, and transportan, arginine homopolymer of from 3 arginine residues to 50 arginine residues. The PTD may be an activatable CPP (ACPP). ACPPs can comprise a polycationic CPP (e.g., Arg9 or “R9”) connected via a cleavable linker to a matching polyanion (e.g., Glu9 or “E9”), which can reduce the net charge to nearly zero and thereby inhibits adhesion and uptake into cells. Upon cleavage of the linker, the polyanion may be released, locally unmasking the polyarginine and its inherent adhesiveness, thus “activating” the ACPP to traverse the membrane.

The present disclosure provides for guide nucleic acids that can direct the activities of an associated polypeptide (e.g., a site-directed polypeptide) to a specific target sequence within a target nucleic acid. The guide nucleic acid may comprise nucleotides. The guide nucleic acid may be RNA. The guide nucleic acid may be DNA. The guide nucleic acid may comprise a single guide nucleic acid. The guide nucleic acid may comprise a spacer extension and/or a tracrRNA extension. The spacer extension and/or tracrRNA extension may comprise elements that contribute additional functionality (e.g., stability) to the guide nucleic acid. In some embodiments the spacer extension and the tracrRNA extension are optional. The guide nucleic acid may comprise a spacer sequence. The spacer sequence may comprise a sequence that hybridizes to a target nucleic acid sequence. The spacer sequence can be a variable portion of the guide nucleic acid. The sequence of the spacer sequence may be engineered to hybridize to the target nucleic acid sequence. The CRISPR repeat (i.e. referred to in this exemplary embodiment as a minimum CRISPR repeat) may comprise nucleotides that can hybridize to a tracrRNA sequence (i.e. referred to in this exemplary embodiment as a minimum tracrRNA sequence). The minimum CRISPR repeat and the minimum tracrRNA sequence may interact, the interacting molecules comprising a base-paired, double-stranded structure. Together, the minimum CRISPR repeat and the minimum tracrRNA sequence may facilitate binding to the site-directed polypeptide. The minimum CRISPR repeat and the minimum tracrRNA sequence may be linked together to form a hairpin structure through the single guide connector. The 3′ tracrRNA sequence may comprise a protospacer adjacent motif recognition sequence. The 3′ tracrRNA sequence may be identical or similar to part of a tracrRNA sequence. In some embodiments, the 3′ tracrRNA sequence may comprise one or more hairpins.

In some embodiments, the guide nucleic acid may comprise a single guide nucleic acid. The guide nucleic acid may comprise a spacer sequence. The spacer sequence may comprise a sequence that can hybridize to the target nucleic acid sequence. The spacer sequence may be a variable portion of the guide nucleic acid. The spacer sequence may be 5′ of a first duplex. The first duplex may comprise a region of hybridization between a minimum CRISPR repeat and minimum tracrRNA sequence. The first duplex may be interrupted by a bulge. The bulge may comprise unpaired nucleotides. The bulge may be facilitate the recruitment of a site-directed polypeptide to the guide nucleic acid. The bulge may be followed by a first stem. The first stem may comprise a linker sequence linking the minimum CRISPR repeat and the minimum tracrRNA sequence. The last paired nucleotide at the 3′ end of the first duplex may be connected to a second linker sequence. The second linker may comprise a P-domain. The second linker may link the first duplex to a mid-tracrRNA. The mid-tracrRNA may, in some embodiments, comprise one or more hairpin regions. For example the mid-tracrRNA may comprise a second stem and a third stem.

In some embodiments, the guide nucleic acid may comprise a double guide nucleic acid structure. Similar to the single guide nucleic acid structure, the double guide nucleic acid structure may comprise a spacer extension, a spacer, a minimum CRISPR repeat, a minimum tracrRNA sequence, a 3′ tracrRNA sequence, and a tracrRNA extension. However, a double guide nucleic acid may not comprise the single guide connector. Instead the minimum CRISPR repeat sequence may comprise a 3′ CRISPR repeat sequence which may be similar or identical to part of a CRISPR repeat. Similarly, the minimum tracrRNA sequence may comprise a 5′ tracrRNA sequence which may be similar or identical to part of a tracrRNA. The double guide RNAs may hybridize together via the minimum CRISPR repeat and the minimum tracrRNA sequence.

In some embodiments, the first segment (i.e., guide segment) may comprise the spacer extension and the spacer. The guide nucleic acid may guide the bound polypeptide to a specific nucleotide sequence within target nucleic acid via the above mentioned guide segment.

In some embodiments, the second segment (i.e., protein binding segment) may comprise the minimum CRISPR repeat, the minimum tracrRNA sequence, the 3′ tracrRNA sequence, and/or the tracrRNA extension sequence. The protein-binding segment of a guide nucleic acid may interact with a site-directed polypeptide. The protein-binding segment of a guide nucleic acid may comprise two stretches of nucleotides that that may hybridize to one another. The nucleotides of the protein-binding segment may hybridize to form a double-stranded nucleic acid duplex. The double-stranded nucleic acid duplex may be RNA. The double-stranded nucleic acid duplex may be DNA.

In some instances, a guide nucleic acid may comprise, in the order of 5′ to 3′, a spacer extension, a spacer, a minimum CRISPR repeat, a single guide connector, a minimum tracrRNA, a 3′ tracrRNA sequence, and a tracrRNA extension. In some instances, a guide nucleic acid may comprise, a tracrRNA extension, a 3′tracrRNA sequence, a minimum tracrRNA, a single guide connector, a minimum CRISPR repeat, a spacer, and a spacer extension in any order.

A guide nucleic acid and a site-directed polypeptide may form a complex. The guide nucleic acid may provide target specificity to the complex by comprising a nucleotide sequence that may hybridize to a sequence of a target nucleic acid. In other words, the site-directed polypeptide may be guided to a nucleic acid sequence by virtue of its association with at least the protein-binding segment of the guide nucleic acid. The guide nucleic acid may direct the activity of a Cas9 protein. The guide nucleic acid may direct the activity of an enzymatically inactive Cas9 protein.

Methods of the disclosure may provide for a genetically modified cell. A genetically modified cell may comprise an exogenous guide nucleic acid and/or an exogenous nucleic acid comprising a nucleotide sequence encoding a guide nucleic acid.

Spacer Extension Sequence

A spacer extension sequence may provide stability and/or provide a location for modifications of a guide nucleic acid. A spacer extension sequence may have a length of from about 1 nucleotide to about 400 nucleotides. A spacer extension sequence may have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 40, 1000, 2000, 3000, 4000, 5000, 6000, or 7000 or more nucleotides. A spacer extension sequence may have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400, 1000, 2000, 3000, 4000, 5000, 6000, 7000 or more nucleotides. A spacer extension sequence may be less than 10 nucleotides in length. A spacer extension sequence may be between 10 and 30 nucleotides in length. A spacer extension sequence may be between 30-70 nucleotides in length.

The spacer extension sequence may comprise a moiety (e.g., a stability control sequence, an endoribonuclease binding sequence, a ribozyme). The moiety may influence the stability of a nucleic acid targeting RNA. The moiety may be a transcriptional terminator segment (i.e., a transcription termination sequence). The moiety of a guide nucleic acid may have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety may be one that may function in a eukaryotic cell. In some cases, the moiety may be one that may function in a prokaryotic cell. The moiety may be one that may function in both a eukaryotic cell and a prokaryotic cell.

Non-limiting examples of suitable moieties may include: 5′ cap (e.g., a 7-methylguanylate cap (m7 G)), a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A spacer extension sequence may comprise a primer binding site, a molecular index (e.g., barcode sequence). The spacer extension sequence may comprise a nucleic acid affinity tag.

Spacer

The guide segment of a guide nucleic acid may comprise a nucleotide sequence (e.g., a spacer) that may hybridize to a sequence in a target nucleic acid. The spacer of a guide nucleic acid may interact with a target nucleic acid in a sequence-specific manner via hybridization (i.e., base pairing). As such, the nucleotide sequence of the spacer may vary and may determine the location within the target nucleic acid that the guide nucleic acid and the target nucleic acid interact.

The spacer sequence may hybridize to a target nucleic acid that is located 5′ of spacer adjacent motif (PAM). Different organisms may comprise different PAM sequences. For example, in S. pyogenes, the PAM may be a sequence in the target nucleic acid that comprises the sequence 5′-XRR-3′, where R may be either A or G, where X is any nucleotide and X is immediately 3′ of the target nucleic acid sequence targeted by the spacer sequence.

The target nucleic acid sequence may be 20 nucleotides. The target nucleic acid may be less than 20 nucleotides. The target nucleic acid may be at least 5, 10, 15, 16, 17, 18, 19, 20, 21, 22, 23, 24, 25, 30 or more nucleotides. The target nucleic acid may be at most 5, 10, 15, 16, 17, 18, 19, 20, 21, 22, 23, 24, 25, 30 or more nucleotides. The target nucleic acid sequence may be 20 bases immediately 5′ of the first nucleotide of the PAM. For example, in a sequence comprising 5′-NNNNNNNNNNNNNNNNNNNNXRR-3′, the target nucleic acid may be the sequence that corresponds to the N's, wherein N is any nucleotide.

The guide sequence of the spacer that may hybridize to the target nucleic acid may have a length at least about 6 nt. For example, the spacer sequence that may hybridize the target nucleic acid may have a length at least about 6 nt, at least about 10 nt, at least about 15 nt, at least about 18 nt, at least about 19 nt, at least about 20 nt, at least about 25 nt, at least about 30 nt, at least about 35 nt or at least about 40 nt, from about 6 nt to about 80 nt, from about 6 nt to about 50 nt, from about 6 nt to about 45 nt, from about 6 nt to about 40 nt, from about 6 nt to about 35 nt, from about 6 nt to about 30 nt, from about 6 nt to about 25 nt, from about 6 nt to about 20 nt, from about 6 nt to about 19 nt, from about 10 nt to about 50 nt, from about 10 nt to about 45 nt, from about 10 nt to about 40 nt, from about 10 nt to about 35 nt, from about 10 nt to about 30 nt, from about 10 nt to about 25 nt, from about 10 nt to about 20 nt, from about 10 nt to about 19 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt. In some cases, the spacer sequence that may hybridize the target nucleic acid may be 20 nucleotides in length. The spacer that may hybridize the target nucleic acid may be 19 nucleotides in length.

The percent complementarity between the spacer sequence the target nucleic acid may be at least about 30%, at least about 40%, at least about 50%, at least about 60%, at least about 65%, at least about 70%, at least about 75%, at least about 80%, at least about 85%, at least about 90%, at least about 95%, at least about 97%, at least about 98%, at least about 99%, or 100%. The percent complementarity between the spacer sequence the target nucleic acid may be at most about 30%, at most about 40%, at most about 50%, at most about 60%, at most about 65%, at most about 70%, at most about 75%, at most about 80%, at most about 85%, at most about 90%, at most about 95%, at most about 97%, at most about 98%, at most about 99%, or 100%. In some cases, the percent complementarity between the spacer sequence and the target nucleic acid may be 100% over the six contiguous 5′-most nucleotides of the target sequence of the complementary strand of the target nucleic acid. In some cases, the percent complementarity between the spacer sequence and the target nucleic acid may be at least 60% over about 20 contiguous nucleotides. In some cases, the percent complementarity between the spacer sequence and the target nucleic acid may be 100% over the fourteen contiguous 5′-most nucleotides of the target sequence of the complementary strand of the target nucleic acid and as low as 0% over the remainder. In such a case, the spacer sequence may be considered to be 14 nucleotides in length. In some cases, the percent complementarity between the spacer sequence and the target nucleic acid may be 100% over the six contiguous 5′-most nucleotides of the target sequence of the complementary strand of the target nucleic acid and as low as 0% over the remainder. In such a case, the spacer sequence may be considered to be 6 nucleotides in length. The target nucleic acid may be more than about 50%, 60%, 70%, 80%, 90%, or 100% complementary to the seed region of the crRNA. The target nucleic acid may be less than about 50%, 60%, 70%, 80%, 90%, or 100% complementary to the seed region of the crRNA.

The spacer segment of a guide nucleic acid may be modified (e.g., by genetic engineering) to hybridize to any desired sequence within a target nucleic acid. For example, a spacer may be engineered (e.g., designed, programmed) to hybridize to a sequence in target nucleic acid that is involved in cancer, cell growth, DNA replication, DNA repair, HLA genes, cell surface proteins, T-cell receptors, immunoglobulin superfamily genes, tumor suppressor genes, microRNA genes, long non-coding RNA genes, transcription factors, globins, viral proteins, mitochondrial genes, and the like.

The spacer sequence may be identified using a computer program (e.g., machine readable code). The computer program may use variables such as predicted melting temperature, secondary structure formation, and predicted annealing temperature, sequence identity, genomic context, chromatin accessibility, % GC, frequency of genomic occurrence, methylation status, presence of SNPs, and the like.

Minimum CRISPR Repeat Sequence

A minimum CRISPR repeat sequence may be a sequence at least about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). The minimum CRISPR repeat sequence may be a sequence with at most about 30%, 40%, 50%, 60%, 65%, 70%, 75%, 80%, 85%, 90%, 95%, or 100% sequence identity and/or sequence homology with a reference CRISPR repeat sequence (e.g., crRNA from S. pyogenes). The minimum CRISPR repeat may comprise nucleotides that may hybridize to a minimum tracrRNA sequence. The minimum CRISPR repeat and a minimum tracrRNA sequence may form a base-paired, double-stranded structure. Together, the minimum CRISPR repeat and the minimum tracrRNA sequence may facilitate binding to the site-directed polypeptide. A part of the minimum CRISPR repeat sequence may hybridize to the minimum tracrRNA sequence. A part of the minimum CRISPR repeat sequence may be at least about 30%, 40%, 50%, 60%, 65%, 70%, 75%, 80%, 85%, 90%, 95%, or 100% complementary to the minimum tracrRNA sequence. A part of the minimum CRISPR repeat sequence may be at most about 30%, 40%, 50%, 60%, 65%, 70%, 75%, 80%, 85%, 90%, 95%, or 100% complementary to the minimum tracrRNA sequence.

The minimum CRISPR repeat sequence may have a length of from about 6 nucleotides to about 100 nucleotides. For example, the minimum CRISPR repeat sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum CRISPR repeat sequence has a length of approximately 12 nucleotides.

The minimum CRISPR repeat sequence may be at least about 60% identical to a reference minimum CRISPR repeat sequence (e.g., wild type crRNA from S. pyogenes) over a stretch of at least 6, 7, or 8 contiguous nucleotides. The minimum CRISPR repeat sequence may be at least about 60% identical to a reference minimum CRISPR repeat sequence (e.g., wild type crRNA from S. pyogenes) over a stretch of at least 6, 7, or 8 contiguous nucleotides. For example, the minimum CRISPR repeat sequence may be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum CRISPR repeat sequence over a stretch of at least 6, 7, or 8 contiguous nucleotides.

Minimum tracrRNA Sequence

A minimum tracrRNA sequence may be a sequence with at least about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). The minimum tracrRNA sequence may be a sequence with at most about 30%, 40%, 50%, 60%, 65%, 70%, 75%, 80%, 85%, 90%, 95%, or 100% sequence identity and/or sequence homology to a reference tracrRNA sequence (e.g., wild type tracrRNA from S. pyogenes). The minimum tracrRNA sequence may comprise nucleotides that may hybridize to a minimum CRISPR repeat sequence. The minimum tracrRNA sequence and a minimum CRISPR repeat sequence may form a base-paired, double-stranded structure. Together, the minimum tracrRNA sequence and the minimum CRISPR repeat may facilitate binding to the site-directed polypeptide. A part of the minimum tracrRNA sequence may hybridize to the minimum CRISPR repeat sequence. A part of the minimum tracrRNA sequence may be 30%, 40%, 50%, 60%, 65%, 70%, 75%, 80%, 85%, 90%, 95%, or 100% complementary to the minimum CRISPR repeat sequence.

The minimum tracrRNA sequence may have a length of from about 6 nucleotides to about 100 nucleotides. For example, the minimum tracrRNA sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the minimum tracrRNA sequence has a length of approximately 14 nucleotides.

The minimum tracrRNA sequence may be at least about 60% identical to a reference minimum tracrRNA (e.g., wild type, tracrRNA from S. pyogenes) sequence over a stretch of at least 6, 7, or 8 contiguous nucleotides. The minimum tracrRNA sequence may be at least about 60% identical to a reference minimum tracrRNA (e.g., wild type, tracrRNA from S. pyogenes) sequence over a stretch of at least 6, 7, or 8 contiguous nucleotides. For example, the minimum tracrRNA sequence may be at least about 65% identical, at least about 70% identical, at least about 75% identical, at least about 80% identical, at least about 85% identical, at least about 90% identical, at least about 95% identical, at least about 98% identical, at least about 99% identical or 100% identical to a reference minimum tracrRNA sequence over a stretch of at least 6, 7, or 8 contiguous nucleotides.

The duplex between the minimum CRISPR RNA and the minimum tracrRNA may comprise a double helix. The first base of the first strand of the duplex may be a guanine. The first base of the first strand of the duplex may be an adenine. The duplex between the minimum CRISPR RNA and the minimum tracrRNA may comprise at least about 1, 2, 3, 4, 5, 6, 7, 8, 9, or 10 or more nucleotides. The duplex between the minimum CRISPR RNA and the minimum tracrRNA may comprise at most about 1, 2, 3, 4, 5, 6, 7, 8, 9, or 10 or more nucleotides.

The duplex may comprise a mismatch. The duplex may comprise at least about 1, 2, 3, 4, or 5 or mismatches. The duplex may comprise at most about 1, 2, 3, 4, or 5 or mismatches. In some instances, the duplex comprises no more than 2 mismatches.

Bulge

A bulge may refer to an unpaired region of nucleotides within the duplex made up of the minimum CRISPR repeat and the minimum tracrRNA sequence. The bulge may be important in the binding to the site-directed polypeptide. A bulge may comprise, on one side of the duplex, an unpaired 5′-XXXY-3′ where X is any purine and Y may be a nucleotide that may form a wobble pair with a nucleotide on the opposite strand, and an unpaired nucleotide region on the other side of the duplex.

For example, the bulge may comprise an unpaired purine (e.g., adenine) on the minimum CRISPR repeat strand of the bulge. In some embodiments, a bulge may comprise an unpaired 5′-AAGY-3′ of the minimum tracrRNA sequence strand of the bulge, where Y may be a nucleotide that may form a wobble pairing with a nucleotide on the minimum CRISPR repeat strand.

A bulge on a first side of the duplex (e.g., the minimum CRISPR repeat side) may comprise at least 1, 2, 3, 4, or 5 or more unpaired nucleotides. A bulge on a first side of the duplex (e.g., the minimum CRISPR repeat side) may comprise at most 1, 2, 3, 4, or 5 or more unpaired nucleotides. A bulge on the first side of the duplex (e.g., the minimum CRISPR repeat side) may comprise 1 unpaired nucleotide.

A bulge on a second side of the duplex (e.g., the minimum tracrRNA sequence side of the duplex) may comprise at least 1, 2, 3, 4, 5, 6, 7, 8, 9, or 10 or more unpaired nucleotides. A bulge on a second side of the duplex (e.g., the minimum tracrRNA sequence side of the duplex) may comprise at most 1, 2, 3, 4, 5, 6, 7, 8, 9, or 10 or more unpaired nucleotides. A bulge on a second side of the duplex (e.g., the minimum tracrRNA sequence side of the duplex) may comprise 4 unpaired nucleotides.

Regions of different numbers of unpaired nucleotides on each strand of the duplex may be paired together. For example, a bulge may comprise 5 unpaired nucleotides from a first strand and 1 unpaired nucleotide from a second strand. A bulge may comprise 4 unpaired nucleotides from a first strand and 1 unpaired nucleotide from a second strand. A bulge may comprise 3 unpaired nucleotides from a first strand and 1 unpaired nucleotide from a second strand. A bulge may comprise 2 unpaired nucleotides from a first strand and 1 unpaired nucleotide from a second strand. A bulge may comprise 1 unpaired nucleotide from a first strand and 1 unpaired nucleotide from a second strand. A bulge may comprise 1 unpaired nucleotide from a first strand and 2 unpaired nucleotides from a second strand. A bulge may comprise 1 unpaired nucleotide from a first strand and 3 unpaired nucleotides from a second strand. A bulge may comprise 1 unpaired nucleotide from a first strand and 4 unpaired nucleotides from a second strand. A bulge may comprise 1 unpaired nucleotide from a first strand and 5 unpaired nucleotides from a second strand.

In some instances a bulge may comprise at least one wobble pairing. In some instances, a bulge may comprise at most one wobble pairing. A bulge sequence may comprise at least one purine nucleotide. A bulge sequence may comprise at least 3 purine nucleotides. A bulge sequence may comprise at least 5 purine nucleotides. A bulge sequence may comprise at least one guanine nucleotide. A bulge sequence may comprise at least one adenine nucleotide.

P-Domain (P-DOMAIN)

A P-domain may refer to a region of a guide nucleic acid that may recognize a protospacer adjacent motif (PAM) in a target nucleic acid. A P-domain may hybridize to a PAM in a target nucleic acid. As such, a P-domain may comprise a sequence that is complementary to a PAM. A P-domain may be located 3′ to the minimum tracrRNA sequence. A P-domain may be located within a 3′ tracrRNA sequence (i.e., a mid-tracrRNA sequence).

A p start at least about 1, 2, 3, 4, 5, 6, 7, 8, 9, 10, 15, or 20 or more nucleotides 3′ of the last paired nucleotide in the minimum CRISPR repeat and minimum tracrRNA sequence duplex. A P-domain may start at most about 1, 2, 3, 4, 5, 6, 7, 8, 9 or 10 or more nucleotides 3′ of the last paired nucleotide in the minimum CRISPR repeat and minimum tracrRNA sequence duplex.

A P-domain may comprise at least about 1, 2, 3, 4, 5, 6, 7, 8, 9, 10, 15, or 20 or more consecutive nucleotides. A P-domain may comprise at most about 1, 2, 3, 4, 5, 6, 7, 8, 9, 10, 15, or 20 or more consecutive nucleotides.

In some instances, a P-domain may comprise a CC dinucleotide (i.e., two consecutive cytosine nucleotides). The CC dinucleotide may interact with the GG dinucleotide of a PAM, wherein the PAM comprises a 5′-XGG-3′ sequence.

A P-domain may be a nucleotide sequence located in the 3′ tracrRNA sequence (i.e., mid-tracrRNA sequence). A P-domain may comprise duplexed nucleotides (e.g., nucleotides in a hairpin, hybridized together. For example, a P-domain may comprise a CC dinucleotide that is hybridized to a GG dinucleotide in a hairpin duplex of the 3′ tracrRNA sequence (i.e., mid-tracrRNA sequence). The activity of the P-domain (e.g., the guide nucleic acid's ability to target a target nucleic acid) may be regulated by the hybridization state of the P-DOMAIN. For example, if the P-domain is hybridized, the guide nucleic acid may not recognize its target. If the P-domain is unhybridized the guide nucleic acid may recognize its target.

The P-domain may interact with P-domain interacting regions within the site-directed polypeptide. The P-domain may interact with an arginine-rich basic patch in the site-directed polypeptide. The P-domain interacting regions may interact with a PAM sequence. The P-domain may comprise a stem loop. The P-domain may comprise a bulge.

3′tracrRNA Sequence

A 3′tracr RNA sequence may be a sequence with at least about 30%, 40%, 50%, 60%, 65%, 70%, 75%, 80%, 85%, 90%, 95%, or 100% sequence identity and/or sequence homology with a reference tracrRNA sequence (e.g., a tracrRNA from S. pyogenes). A 3′tracr RNA sequence may be a sequence with at most about 30%, 40%, 50%, 60%, 65%, 70%, 75%, 80%, 85%, 90%, 95%, or 100% sequence identity and/or sequence homology with a reference tracrRNA sequence (e.g., tracrRNA from S. pyogenes).

The 3′ tracrRNA sequence may have a length of from about 6 nucleotides to about 100 nucleotides. For example, the 3′ tracrRNA sequence may have a length of from about 6 nucleotides (nt) to about 50 nt, from about 6 nt to about 40 nt, from about 6 nt to about 30 nt, from about 6 nt to about 25 nt, from about 6 nt to about 20 nt, from about 6 nt to about 15 nt, from about 8 nt to about 40 nt, from about 8 nt to about 30 nt, from about 8 nt to about 25 nt, from about 8 nt to about 20 nt or from about 8 nt to about 15 nt, from about 15 nt to about 100 nt, from about 15 nt to about 80 nt, from about 15 nt to about 50 nt, from about 15 nt to about 40 nt, from about 15 nt to about 30 nt or from about 15 nt to about 25 nt. In some embodiments, the 3′ tracrRNA sequence has a length of approximately 14 nucleotides.

The 3′ tracrRNA sequence may be at least about 60% identical to a reference 3′ tracrRNA sequence (e.g., wild type 3′ tracrRNA sequence from S. pyogenes) over a stretch of at least 6, 7, or 8 contiguous nucleotides. For example, the 3′ tracrRNA sequence may be at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical, to a reference 3′ tracrRNA sequence (e.g., wild type 3′ tracrRNA sequence from S. pyogenes) over a stretch of at least 6, 7, or 8 contiguous nucleotides.

A 3′ tracrRNA sequence may comprise more than one duplexed region (e.g., hairpin, hybridized region). A 3′ tracrRNA sequence may comprise two duplexed regions.

The 3′ tracrRNA sequence may also be referred to as the mid-tracrRNA. The mid-tracrRNA sequence may comprise a stem loop structure. In other words, the mid-tracrRNA sequence may comprise a hairpin that is different than a second or third stems. A stem loop structure in the mid-tracrRNA (i.e., 3′ tracrRNA) may comprise at least 1, 2, 3, 4, 5, 6, 7, 8, 9, 10, 15 or 20 or more nucleotides. A stem loop structure in the mid-tracrRNA (i.e., 3′ tracrRNA) may comprise at most 1, 2, 3, 4, 5, 6, 7, 8, 9 or 10 or more nucleotides. The stem loop structure may comprise a functional moiety. For example, the stem loop structure may comprise an aptamer, a ribozyme, a protein-interacting hairpin, a CRISPR array, an intron, and an exon. The stem loop structure may comprise at least about 1, 2, 3, 4, or 5 or more functional moieties. The stem loop structure may comprise at most about 1, 2, 3, 4, or 5 or more functional moieties.

The hairpin in the mid-tracrRNA sequence may comprise a P-domain. The P-domain may comprise a double stranded region in the hairpin.

tracrRNA Extension Sequence

A tracrRNA extension sequence may provide stability and/or provide a location for modifications of a guide nucleic acid. The tracrRNA extension sequence may have a length of from about 1 nucleotide to about 400 nucleotides. The tracrRNA extension sequence may have a length of more than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 or more nucleotides. The tracrRNA extension sequence may have a length from about 20 to about 5000 or more nucleotides. The tracrRNA extension sequence may have a length of more than 1000 nucleotides. The tracrRNA extension sequence may have a length of less than 1, 5, 10, 15, 20, 25, 30, 35, 40, 45, 50, 60, 70, 80, 90, 100, 120, 140, 160, 180, 200, 220, 240, 260, 280, 300, 320, 340, 360, 380, 400 nucleotides. The tracrRNA extension sequence may have a length of less than 1000 nucleotides. The tracrRNA extension sequence may be less than 10 nucleotides in length. The tracrRNA extension sequence may be between 10 and 30 nucleotides in length. The tracrRNA extension sequence may be between 30-70 nucleotides in length.

The tracrRNA extension sequence may comprise a moiety (e.g., stability control sequence, ribozyme, endoribonuclease binding sequence). A moiety may influence the stability of a nucleic acid targeting RNA. A moiety may be a transcriptional terminator segment (i.e., a transcription termination sequence). A moiety of a guide nucleic acid may have a total length of from about 10 nucleotides to about 100 nucleotides, from about 10 nucleotides (nt) to about 20 nt, from about 20 nt to about 30 nt, from about 30 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt, from about 15 nucleotides (nt) to about 80 nt, from about 15 nt to about 50 nt, from about 15 nt to about 40 nt, from about 15 nt to about 30 nt or from about 15 nt to about 25 nt. The moiety may be one that may function in a eukaryotic cell. In some cases, the moiety may be one that may function in a prokaryotic cell. The moiety may be one that may function in both a eukaryotic cell and a prokaryotic cell.

Non-limiting examples of suitable tracrRNA extension moieties include: a 3′ poly-adenylated tail, a riboswitch sequence (e.g., to allow for regulated stability and/or regulated accessibility by proteins and protein complexes), a sequence that forms a dsRNA duplex (i.e., a hairpin), a sequence that targets the RNA to a subcellular location (e.g., nucleus, mitochondria, chloroplasts, and the like), a modification or sequence that provides for tracking (e.g., direct conjugation to a fluorescent molecule, conjugation to a moiety that facilitates fluorescent detection, a sequence that allows for fluorescent detection, etc.), a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like) a modification or sequence that provides for increased, decreased, and/or controllable stability, or any combination thereof. A tracrRNA extension sequence may comprise a primer binding site, a molecular index (e.g., barcode sequence). In some embodiments of the disclosure, the tracrRNA extension sequence may comprise one or more affinity tags.

Single Guide Nucleic Acid

The guide nucleic acid may be a single guide nucleic acid. The single guide nucleic acid may be RNA. A single guide nucleic acid may comprise a linker between the minimum CRISPR repeat sequence and the minimum tracrRNA sequence that may be called a single guide connector sequence.

The single guide connector of a single guide nucleic acid may have a length of from about 3 nucleotides to about 100 nucleotides. For example, the linker may have a length of from about 3 nucleotides (nt) to about 90 nt, from about 3 nt to about 80 nt, from about 3 nt to about 70 nt, from about 3 nt to about 60 nt, from about 3 nt to about 50 nt, from about 3 nt to about 40 nt, from about 3 nt to about 30 nt, from about 3 nt to about 20 nt or from about 3 nt to about 10 nt. For example, the linker may have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single guide nucleic acid is between 4 and 40 nucleotides. The linker may have a length at least about 100, 500, 1000, 1500, 2000, 2500, 3000, 3500, 4000, 4500, 5000, 5500, 6000, 6500, or 7000 or more nucleotides. The linker may have a length at most about 100, 500, 1000, 1500, 2000, 2500, 3000, 3500, 4000, 4500, 5000, 5500, 6000, 6500, or 7000 or more nucleotides.

The linker sequence may comprise a functional moiety. For example, the linker sequence may comprise an aptamer, a ribozyme, a protein-interacting hairpin, a CRISPR array, an intron, and an exon. The linker sequence may comprise at least about 1, 2, 3, 4, or 5 or more functional moieties. The linker sequence may comprise at most about 1, 2, 3, 4, or 5 or more functional moieties.

In some embodiments, the single guide connector may connect the 3′ end of the minimum CRISPR repeat to the 5′ end of the minimum tracrRNA sequence. Alternatively, the single guide connector may connect the 3′ end of the tracrRNA sequence to the 5′ end of the minimum CRISPR repeat. That is to say, a single guide nucleic acid may comprise a 5′ DNA-binding segment linked to a 3′ protein-binding segment. A single guide nucleic acid may comprise a 5′ protein-binding segment linked to a 3′ DNA-binding segment.

The guide nucleic acid may comprise a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6, 7, or 8 contiguous nucleotides and wherein the minimum CRISPR repeat has a length from 5-30 nucleotides; a minimum tracrRNA sequence comprising at least 60% identity to a tracrRNA from a bacterium (e.g., S. pyogenes) over 6, 7, or 8 contiguous nucleotides and wherein the minimum tracrRNA sequence has a length from 5-30 nucleotides; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6, 7, or 8 contiguous nucleotides and wherein the 3′ tracrRNA comprises a length from 10-20 nucleotides, and comprises a duplexed region; and/or a tracrRNA extension comprising 10-5000 nucleotides in length, or any combination thereof. This guide nucleic acid may be referred to as a single guide nucleic acid.

The guide nucleic acid may comprise a spacer extension sequence from 10-5000 nucleotides in length; a spacer sequence of 12-30 nucleotides in length, wherein the spacer is at least 50% complementary to a target nucleic acid; a duplex comprising 1) a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, 2) a minimum tracrRNA sequence comprising at least 60% identity to a tracrRNA from a bacterium (e.g., S. pyogenes) over 6 contiguous nucleotides and wherein the minimum tracrRNA sequence has a length from 5-30 nucleotides, and 3) a bulge wherein the bulge comprises at least 3 unpaired nucleotides on the minimum CRISPR repeat strand of the duplex and at least 1 unpaired nucleotide on the minimum tracrRNA sequence strand of the duplex; a linker sequence that links the minimum CRISPR repeat and the minimum tracrRNA and comprises a length from 3-5000 nucleotides; a 3′ tracrRNA that comprises at least 60% identity to a tracrRNA from a prokaryote (e.g., S. pyogenes) or phage over 6 contiguous nucleotides, wherein the 3′ tracrRNA comprises a length from 10-20 nucleotides and comprises a duplexed region; a P-domain that starts from 1-5 nucleotides downstream of the duplex comprising the minimum CRISPR repeat and the minimum tracrRNA, comprises 1-10 nucleotides, comprises a sequence that may hybridize to a protospacer adjacent motif in a target nucleic acid, may form a hairpin, and is located in the 3′ tracrRNA region; and/or a tracrRNA extension comprising 10-5000 nucleotides in length, or any combination thereof.

Double Guide Nucleic Acid

The guide nucleic acid may be a double guide nucleic acid. The double guide nucleic acid can be RNA. The double guide nucleic acid can comprise two separate nucleic acid molecules (i.e. polynucleotides). Each of the two nucleic acid molecules of a double guide nucleic acid can comprise a stretch of nucleotides that can hybridize to one another such that the complementary nucleotides of the two nucleic acid molecules hybridize to form the double stranded duplex of the protein-binding segment. If not otherwise specified, the term “guide nucleic acid” can be inclusive, referring to both single-molecule guide nucleic acids and double-molecule guide nucleic acids.

The double guide nucleic acid may comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; and a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides; and 2) a second nucleic acid molecule of the double-guide nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides; a 3′ tracrRNA that comprises at least 60% identity to a tracrRNA from a bacterium (e.g., S. pyogenes) over 6 contiguous nucleotides and wherein the 3′ tracrRNA comprises a length from 10-20 nucleotides, and comprises a duplexed region; and/or a tracrRNA extension comprising 10-5000 nucleotides in length, or any combination thereof.

In some instances, the double-guide nucleic acid may comprise 1) a first nucleic acid molecule comprising a spacer extension sequence from 10-5000 nucleotides in length; a spacer sequence of 12-30 nucleotides in length, wherein the spacer is at least 50% complementary to a target nucleic acid; a minimum CRISPR repeat comprising at least 60% identity to a crRNA from a prokaryote (e.g., S. pyogenes) or phage over 6 contiguous nucleotides and wherein the minimum CRISPR repeat has a length from 5-30 nucleotides, and at least 3 unpaired nucleotides of a bulge; and 2) a second nucleic acid molecule of the double-guide nucleic acid can comprise a minimum tracrRNA sequence comprising at least 60% identity to a tracrRNA from a prokaryote (e.g., S. pyogenes) or phage over 6 contiguous nucleotides and wherein the minimum tracrRNA sequence has a length from 5-30 nucleotides and at least 1 unpaired nucleotide of a bulge, wherein the lunpaired nucleotide of the bulge is located in the same bulge as the 3 unpaired nucleotides of the minimum CRISPR repeat; a 3′ tracrRNA that comprises at least 60% identity to a tracrRNA from a prokaryote (e.g., S. pyogenes) or phage over 6 contiguous nucleotides and wherein the 3′ tracrRNA comprises a length from 10-20 nucleotides, and comprises a duplexed region; a P-domain that starts from 1-5 nucleotides downstream of the duplex comprising the minimum CRISPR repeat and the minimum tracrRNA, comprises 1-10 nucleotides, comprises a sequence that can hybridize to a protospacer adjacent motif in a target nucleic acid, can form a hairpin, and is located in the 3′ tracrRNA region; and/or a tracrRNA extension comprising 10-5000 nucleotides in length, or any combination thereof.

Complex of a Guide Nucleic Acid and a Site-Directed Polypeptide

The guide nucleic acid may interact with a site-directed polypeptide (e.g., a nucleic acid-guided nucleases, Cas9), thereby forming a complex. The guide nucleic acid may guide the site-directed polypeptide to a target nucleic acid.

In some embodiments, the guide nucleic acid may be engineered such that the complex (e.g., comprising a site-directed polypeptide and a guide nucleic acid) can bind outside of the cleavage site of the site-directed polypeptide. In this case, the target nucleic acid may not interact with the complex and the target nucleic acid can be excised (e.g., free from the complex).

In some embodiments, the guide nucleic acid may be engineered such that the complex can bind inside of the cleavage site of the site-directed polypeptide. In this case, the target nucleic acid can interact with the complex and the target nucleic acid can be bound (e.g., bound to the complex).

Any guide nucleic acid of the disclosure, a site-directed polypeptide of the disclosure, an effector protein, a multiplexed genetic targeting agent, a donor polynucleotide, a tandem fusion protein, a reporter element, a genetic element of interest, a component of a split system and/or any nucleic acid or proteinaceous molecule necessary to carry out the embodiments of the methods of the disclosure may be recombinant, purified and/or isolated.

In some embodiments, the methods comprise using a CRISPR/Cas system to modify a mutation in the nucleic acid molecule. In some embodiments, the mutation is a substitution, insertion, or deletion. In some embodiments, the mutation is a single nucleotide polymorphism.

In some cases, the target sequence is between 10 to 30 nucleotides in length. In some instances, the target sequence is between 15 to 30 nucleotides in length. In some cases, the target sequence is about 11, 12, 13, 14, 15, 16, 17, 18, 19, 20, 21, 22, 23, 24, 25, 26, 27, 28, 29, or 30 nucleotides in length. In some cases, the target sequence is about 15, 16, 17, 18, 19, 20, 21, or 22 nucleotides in length.

In some instances, a CRISPR/Cas system utilizes a Cas9 enzyme or a variant thereof. In some embodiments, the methods and cell disclosed herein utilize a polynucleotide encoding the Cas9 enzyme or the variant thereof. In some embodiments, the Cas9 is a double stranded nuclease with two active cutting sites, one for each strand of the double helix. In some instances, the Cas9 enzyme or variant thereof generates a double-stranded break. In some embodiments, the Cas9 enzyme is a wildtype Cas9 enzyme. In some embodiments, the Cas9 enzyme is a naturally-occurring variant or mutant of the wildtype Cas9 enzyme or S. pyogenes Cas9 enzyme. The variant may be an enzyme that is partially homologous to a wildtype Cas9 enzyme, while maintaining Cas9 nuclease activity. The variant may be an enzyme that only comprises a portion of the wildtype Cas9 enzyme, while maintaining Cas9 nuclease activity. In some embodiments, the wildtype Cas9 enzyme is a Streptococcus pyogenes (S. pyogenes) Cas9 enzyme. In some embodiments, the wildtype Cas9 enzyme is represented by an amino acid sequence given GenBank ID AKP81606.1. In some embodiments, the variant is at least about 95% homologous to the amino acid sequence given GenBank ID AKP81606.1. In some embodiments, the variant is at least about 90% homologous to the amino acid sequence given GenBank ID AKP81606.1. In some embodiments, the variant is at least about 80% homologous to the amino acid sequence given GenBank ID AKP81606.1. In some embodiments, the variant is at least about 70% homologous to the amino acid sequence given GenBank ID AKP81606.1. In some instances, the Cas9 enzyme is an optimized Cas9 enzyme, modified from the wild-type Cas9 enzyme for optimal expression and/or activity in the cells described herein. In some embodiments, the Cas9 enzyme is a modified Cas9 enzyme, wherein the modified Cas9 enzyme comprises a Cas9 enzyme or variant thereof as described herein and an additional amino acid sequence. The additional amino acid sequence, by way of non-limiting example, may provide an additional activity, stability, or identifying tag/barcode to the Cas9 enzyme or variant thereof.

The naturally-occurring S. pyogenes Cas9 enzyme cleaves DNA to generate a double stranded break. In some embodiments, the Cas9 enzymes disclosed herein function as a Cas9 nickase, wherein the Cas9 nickase is a Cas9 enzyme that has been modified to nick the target sequence, creating a single stranded break. In some embodiments, the methods disclosed herein comprise use of the Cas9 nickase with more than one guide RNA targeting the target sequence to cleave each DNA strand in a staggered pattern at the target sequence. In some embodiments, using two guide RNAs with Cas9 nickase may increase the target specificity of the CRISPR/Cas systems disclosed herein. In some embodiments, using two or more guide RNAs may result in generating a genomic deletion. In some embodiments, the genomic deletion is a deletion of about 5 nucleotides to about 50,000 nucleotides. In some embodiments, the genomic deletion is a deletion of about 5 nucleotides to about 1,000 nucleotides. In some embodiments, the methods disclosed herein comprise using a plurality of guide RNAs. In some embodiments, the plurality of guide RNAs targets a single gene. In some embodiments, the plurality of guide RNAs targets a plurality of genes.

In some instances, the specificity of the guide RNA for the target sequence is about 80%, 85%, 90%, 95%, 96%, 97%, 98%, 99%, or higher. In some instances, the guide RNA has less than about 20%, 15%, 10%, 5%, 3%, 1%, or less off-target binding rate.

In some embodiments, the specificity of the guide RNA that hybridizes to the target sequence has about 95%, 98%, 99%, 99.5% or 100% sequence complementarity to the target sequence. In some instances, the hybridization is a high stringent hybridization condition.

In some embodiments, the guide RNA targets the nuclease to a gene encoding a neural retina leucine zipper (NRL) protein. In some embodiments, the guide RNA comprises a sequence that hybridizes to a target sequence of the NRL encoding gene. In some embodiments, the target sequence selected from SEQ ID NOS: 1-2. In some embodiments, the target sequence is at least 90% homologous to a sequence selected from SEQ ID NOS: 1-2. In some embodiments, the target sequence is at least about 80% homologous to a sequence selected from SEQ ID NOS: 1-2. In some embodiments, the target sequence is at least about 85% homologous to a sequence selected from SEQ ID NOS: 1-2. In some embodiments, the target sequence is at least about 90% homologous to a sequence selected from SEQ ID NOS: 1-2. In some embodiments, the target sequence is at least about 95% homologous to a sequence selected from SEQ ID NOS: 1-2.

In some embodiments, the guide RNA targets the nuclease to a gene encoding a nuclear receptor subfamily 2 group E member 3 (NR2E3) protein. In some embodiments, the guide RNA comprises a sequence that hybridizes to a target sequence of the NR2E3 encoding gene. In some embodiments, the target sequence selected from SEQ ID NOS: 3-4. In some embodiments, the target sequence is at least 90% homologous to a sequence selected from SEQ ID NOS: 3-4. In some embodiments, the target sequence is at least about 80% homologous to a sequence selected from SEQ ID NOS: 3-4. In some embodiments, the target sequence is at least about 85% homologous to a sequence selected from SEQ ID NOS: 3-4. In some embodiments, the target sequence is at least about 90% homologous to a sequence selected from SEQ ID NOS: 3-4. In some embodiments, the target sequence is at least about 95% homologous to a sequence selected from SEQ ID NOS: 3-4.

DNA-Guided Nucleases

In some embodiments, methods and cells disclosed herein utilize a nucleic acid-guided nuclease system. In some embodiments, the methods and cells disclosed herein use DNA-guided nuclease systems. In some embodiments, the methods and cells disclosed herein use Argonaute systems.

An Argonaute protein may be a polypeptide that can bind to a target nucleic acid. The Argonaute protein may be a nuclease. The Argonaute protein may be a eukaryotic, prokaryotic, or archaeal Argonaute protein. The Argonaute protein may be a prokaryotic Argonaute protein (pArgonaute). The pArgonaute may be derived from an archaea. The pArgonaute may be derived from a bacterium. The bacterium may be selected from a thermophilic bacterium and a mesophilic bacterium. The bacteria or archaea may be selected from Aquifex aeolicus, Microsystis aeruginosa, Clostridium bartlettii, Exiguobacterium, Anoxybacillus flavithermus, Halogeometricum borinquense, Halorubrum lacusprofundi, Aromatoleum aromaticum, Thermus thermophilus, Synechococcus, Synechococcus elongatus, and Thermosynechococcus elogatus, or any combination thereof. The bacterium may be a thermophilic bacterium. The bacterium may be Aquifex aeolicus. The thermophilic bacterium may be Thermus thermophilus (T. thermophilus) (TtArgonaute). The Argonaute may be from a Synechococcus bacterium. The Argonaute may be from Synechococcus elongatus. The pArgonaute may be a variant pArgonaute of a wild-type pArgonaute.

In some embodiments, the Argonaute of the disclosure is a type I prokaryotic Argonaute (pAgo). In some embodiments, the type I prokaryotic Argonaute carries a DNA nucleic acid-targeting nucleic acid. In some embodiments, the DNA nucleic acid-targeting nucleic acid targets one strand of a double stranded DNA (dsDNA) to produce a nick or a break of the dsDNA. In some embodiments, the nick or break triggers host DNA repair. In some embodiments, the host DNA repair is non-homologous end joining (NHEJ) or homologous directed recombination (HDR). In some embodiments, the dsDNA is selected from a genome, a chromosome and a plasmid. In some embodiments, the type I prokaryotic Argonaute is a long type I prokaryotic Argonaute. In some embodiments, the long type I prokaryotic Argonaute possesses an N-PAZ-MID-PIWI domain architecture. In some embodiments the long type I prokaryotic Argonaute possesses a catalytically active PIWI domain. In some embodiments, the long type I prokaryotic Argonaute possesses a catalytic tetrad encoded by aspartate-glutamate-aspartate-aspartate/histidine (DEDX). In some embodiments, the catalytic tetrad binds one or more Mg+ ions. In some embodiments, the catalytic tetrad does not bind Mg+ ions. In some embodiments, the catalytic tetrad binds one or more Mn+ ions. In some embodiments, the catalytically active PIWI domain is optimally active at a moderate temperature. In some embodiments, the moderate temperature is about 25° C. to about 45° C. In some embodiments, the moderate temperature is about 37° C. In some embodiments, the type I prokaryotic Argonaute anchors the 5′ phosphate end of a DNA guide. In some embodiments, the DNA guide has a deoxy-cytosine at its 5′ end. In some embodiments, the type I prokaryotic Argonaute is a Thermus thermophilus Ago (TtAgo). In some embodiments, the type I prokaryotic Argonaute is a Synechococcus elongatus Ago (SeAgo).

In some embodiments, the prokaryotic Argonaute is a type II pAgo. In some embodiments, the type II prokaryotic Argonaute carries an RNA nucleic acid-targeting nucleic acid. In some embodiments, the RNA nucleic acid-targeting nucleic acid targets one strand of a double stranded DNA (dsDNA) to produce a nick or a break of the dsDNA. In some embodiments, the nick or break triggers host DNA repair. In some embodiments, the host DNA repair is non-homologous end joining (NHEJ) or homologous directed recombination (HDR). In some embodiments, the dsDNA is selected from a genome, a chromosome and a plasmid. In some embodiments, the type II prokaryotic Argonaute is selected from a long type II prokaryotic Argonaute and a short type II prokaryotic Argonaute. In some embodiments, the long type II prokaryotic Argonaute has an N-PAZ-MID-PIWI domain architecture. In some embodiments, the long type II prokaryotic Argonaute does not have an N-PAZ-MID-PIWI domain architecture. In some embodiments, the short type II prokaryotic Argonaute has a MID and PIWI domain, but not a PAZ domain. In some embodiments, the short type II pAgo has an analog of a PAZ domain. In some embodiments the type II pAgo does not have a catalytically active PIWI domain. In some embodiments, the type II pAgo lacks a catalytic tetrad encoded by aspartate-glutamate-aspartate-aspartate/histidine (DEDX). In some embodiments, a gene encoding the type II prokaryotic Argonaute clusters with one or more genes encoding a nuclease, a helicase or a combination thereof. The nuclease or helicase may be natural, designed or a domain thereof. In some embodiments, the nuclease is selected from a Sir2, RE1 and TIR. In some embodiments, the type II pAgo anchors the 5′ phosphate end of an RNA guide. In some embodiments, the RNA guide has a uracil at its 5′ end. In some embodiments, the type II prokaryotic Argonaute is a Rhodobacter sphaeroides Argonaute (RsAgo).

In some embodiments, a pair of pAgos can carry RNA and/or DNA nucleic acid-targeting nucleic acid. A type I pAgo can carry an RNA nucleic acid-targeting nucleic acid, each capable of targeting one strand of a double stranded DNA to produce a double-stranded break in the double stranded DNA. In some embodiments, the pair of pAgos comprises two types I pAgos. In some embodiments, the pair of pAgos comprises two type II pAgos. In some embodiments, the pair of pAgos comprises a type I pAgo and a type II pAgo.

Argonaute proteins can be targeted to target nucleic acid sequences by a guiding nucleic acid.

The guiding nucleic acid can be single stranded or double stranded. The guiding nucleic acid can be DNA, RNA, or a DNA/RNA hybrid. The guiding nucleic acid can comprise chemically modified nucleotides.

The guiding nucleic acid can hybridize with the sense or antisense strand of a target polynucleotide.

The guiding nucleic acid can have a 5′ modification. 5′ modifications can be phosphorylation, methylation, hydroxymethylation, acetylation, ubiquitylation, or sumolyation. The 5′ modification can be phosphorylation.

The guiding nucleic acid can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs in length. In some examples, the guiding nucleic acid can be less than 10 nucleotides or base pairs in length. In some examples, the guiding nucleic acid can be more than 50 nucleotides or base pairs in length.

The guiding nucleic acid can be a guide DNA (gDNA). The gDNA can have a 5′ phosphorylated end. The gDNA can be single stranded or double stranded. The gDNA can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides or base pairs in length. In some examples, the gDNA can be less than 10 nucleotides in length. In some examples, the gDNA can be more than 50 nucleotides in length.

### Multiplexing

Disclosed herein are methods, compositions, systems, and/or kits for multiplexed genome engineering. In some embodiments of the disclosure a site-directed polypeptide may comprise a guide nucleic acid, thereby forming a complex. The complex may be contacted with a target nucleic acid. The target nucleic acid may be cleaved, and/or modified by the complex. The methods, compositions, systems, and/or kits of the disclosure may be useful in modifying multiple target nucleic acids quickly, efficiently, and/or simultaneously. The method may be performed using any of the site-directed polypeptides (e.g., Cas9), guide nucleic acids, and complexes of site-directed polypeptides and guide nucleic acids as described herein.

Site-directed nucleases of the disclosure may be combined in any combination. For example, multiple CRISPR/Cas nucleases may be used to target different target sequences or different segments of the same target. In another example, Cas9 and Argonaute may be used in combination to target different targets or different sections of the same target. In some embodiments, a site-directed nuclease may be used with multiple different guide nucleic acids to target multiple different sequences simultaneously.

A nucleic acid (e.g., a guide nucleic acid) may be fused to a non-native sequence (e.g., a moiety, an endoribonuclease binding sequence, ribozyme), thereby forming a nucleic acid module. The nucleic acid module (e.g., comprising the nucleic acid fused to a non-native sequence) may be conjugated in tandem, thereby forming a multiplexed genetic targeting agent (e.g., polymodule, e.g., array). The multiplexed genetic targeting agent may comprise RNA. The multiplexed genetic targeting agent may be contacted with one or more endoribonucleases. The endoribonucleases may bind to the non-native sequence. The bound endoribonuclease may cleave a nucleic acid module of the multiplexed genetic targeting agent at a prescribed location defined by the non-native sequence. The cleavage may process (e.g., liberate) individual nucleic acid modules. In some embodiments, the processed nucleic acid modules may comprise all, some, or none, of the non-native sequence. The processed nucleic acid modules may be bound by a site-directed polypeptide, thereby forming a complex. The complex may be targeted to a target nucleic acid. The target nucleic acid may by cleaved and/or modified by the complex.

A multiplexed genetic targeting agent may be used in modifying multiple target nucleic acids at the same time, and/or in stoichiometric amounts. A multiplexed genetic targeting agent may be any nucleic acid-targeting nucleic acid as described herein in tandem. A multiplexed genetic targeting agent may refer to a continuous nucleic acid molecule comprising one or more nucleic acid modules. A nucleic acid module may comprise a nucleic acid and a non-native sequence (e.g., a moiety, endoribonuclease binding sequence, ribozyme). The nucleic acid may be non-coding RNA such as microRNA (miRNA), short interfering RNA (siRNA), long non-coding RNA (lncRNA, or lincRNA), endogenous siRNA (endo-siRNA), piwi-interacting RNA (piRNA), trans-acting short interfering RNA (tasiRNA), repeat-associated small interfering RNA (rasiRNA), small nucleolar RNA (snoRNA), small nuclear RNA (snRNA), transfer RNA (tRNA), and ribosomal RNA (rRNA), or any combination thereof. The nucleic acid may be a coding RNA (e.g., a mRNA). The nucleic acid may be any type of RNA. In some embodiments, the nucleic acid may be a nucleic acid-targeting nucleic acid.

The non-native sequence may be located at the 3′ end of the nucleic acid module. The non-native sequence may be located at the 5′ end of the nucleic acid module. The non-native sequence may be located at both the 3′ end and the 5′ end of the nucleic acid module. The non-native sequence may comprise a sequence that may bind to a endoribonuclease (e.g., endoribonuclease binding sequence). The non-native sequence may be a sequence that is sequence-specifically recognized by an endoribonuclease (e.g., RNase T1 cleaves unpaired G bases, RNase T2 cleaves 3′end of As, RNase U2 cleaves 3′end of unpaired A bases). The non-native sequence may be a sequence that is structurally recognized by an endoribonuclease (e.g., hairpin structure, single-stranded-double stranded junctions, e.g., Drosha recognizes a single-stranded-double stranded junction within a hairpin). The non-native sequence may comprise a sequence that may bind to a CRISPR system endoribonuclease (e.g., Csy4, Cas5, and/or Cas6 protein).

In some embodiments, wherein the non-native sequence comprises an endoribonuclease binding sequence, the nucleic acid modules may be bound by the same endoribonuclease. The nucleic acid modules may not comprise the same endoribonuclease binding sequence. The nucleic acid modules may comprise different endoribonuclease binding sequences. The different endoribonuclease binding sequences may be bound by the same endoribonuclease. In some embodiments, the nucleic acid modules may be bound by different endoribonucleases.

The moiety may comprise a ribozyme. The ribozyme may cleave itself, thereby liberating each module of the multiplexed genetic targeting agent. Suitable ribozymes may include peptidyl transferase 23S rRNA, RnaseP, Group I introns, Group II introns, GIR1 branching ribozyme, Leadzyme, hairpin ribozymes, hammerhead ribozymes, HDV ribozymes, CPEB3 ribozymes, VS ribozymes, glmS ribozyme, CoTC ribozyme, an synthetic ribozymes.

The nucleic acids of the nucleic acid modules of the multiplexed genetic targeting agent may be identical. The nucleic acid modules may differ by 1, 2, 3, 4, 5, 6, 7, 8, 9, 10, 15, 20, 25, 30, 35, 40, 45, 50 or more nucleotides. For example, different nucleic acid modules may differ in the spacer region of the nucleic acid module, thereby targeting the nucleic acid module to a different target nucleic acid. In some instances, different nucleic acid modules may differ in the spacer region of the nucleic acid module, yet still target the same target nucleic acid. The nucleic acid modules may target the same target nucleic acid. The nucleic acid modules may target one or more target nucleic acids.

A nucleic acid module may comprise a regulatory sequence that may allow for appropriate translation or amplification of the nucleic acid module. For example, an nucleic acid module may comprise a promoter, a TATA box, an enhancer element, a transcription termination element, a ribosome-binding site, a 3′ un-translated region, a 5′ un-translated region, a 5′ cap sequence, a 3′ poly adenylation sequence, an RNA stability element, and the like.

Nucleic Acids Encoding a Designed Guide Nucleic Acid and/or Nucleic-Acid Guided Nuclease

The present disclosure provides for a nucleic acid comprising a nucleotide sequence encoding a guide nucleic acid of the disclosure, an nucleic-acid guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure. In some embodiments, a nucleic acid encoding a guide nucleic acid of the disclosure, an nucleic-acid guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be a vector (e.g., a recombinant expression vector).

In some embodiments, the recombinant expression vector may be a viral construct, (e.g., a recombinant adeno-associated virus construct), a recombinant adenoviral construct, a recombinant lentiviral construct, a recombinant retroviral construct, etc.

Suitable expression vectors may include, but are not limited to, viral vectors (e.g. viral vectors based on vaccinia virus, poliovirus, adenovirus, adeno-associated virus, SV40, herpes simplex virus, human immunodeficiency virus, a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus), plant vectors (e.g., T-DNA vector), and the like. The following vectors may be provided by way of example, for eukaryotic host cells: pXT1, pSG5, pSVK3, pBPV, pMSG, and pSVLSV40 (Pharmacia). Other vectors may be used so long as they are compatible with the host cell.

In some instances, the vector may be a linearized vector. The linearized vector may comprise a nuclease (e.g. Cas9 or Argonaute) and/or a guide nucleic acid. The linearized vector may not be a circular plasmid. The linearized vector may comprise a double-stranded break. The linearized vector may comprise a sequence encoding a fluorescent protein (e.g., orange fluorescent protein (OFP)). The linearized vector may comprise a sequence encoding an antigen (e.g., CD4). The linearized vector may be linearized (e.g., cut) in a region of the vector encoding parts of the designed nucleic acid-targeting nucleic acid. For example the linearized vector may be linearized (e.g., cut) in a 5′ region of the designed nucleic acid-targeting nucleic acid. The linearized vector may be linearized (e.g., cut) in a 3′ region of the designed nucleic acid-targeting nucleic acid. In some instances, a linearized vector or a closed supercoiled vector comprises a sequence encoding a nuclease (e.g., Cas9 or Argonaute), a promoter driving expression of the sequence encoding the nuclease (e.g., CMV promoter), a sequence encoding a marker, a sequence encoding an affinity tag, a sequence encoding portion of a guide nucleic acid, a promoter driving expression of the sequence encoding a portion of the guide nucleic acid, and a sequence encoding a selectable marker (e.g., ampicillin), or any combination thereof.

The vector may comprise a transcription and/or translation control element. Depending on the host/vector system utilized, any of a number of suitable transcription and translation control elements, including constitutive and inducible promoters, transcription enhancer elements, transcription terminators, etc. may be used in the expression vector.

In some embodiments, a nucleotide sequence encoding a guide nucleic acid of the disclosure, an nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to a control element (e.g., a transcriptional control element), such as a promoter. The transcriptional control element may be functional in a eukaryotic cell, (e.g., a mammalian cell), and/or a prokaryotic cell (e.g., bacterial or archaeal cell). In some embodiments, a nucleotide sequence encoding a designed guide nucleic acid of the disclosure, a nucleic acid-guided nuclease (e.g., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to multiple control elements. Operable linkage to multiple control elements may allow expression of the nucleotide sequence encoding a guide nucleic acid of the disclosure, a nucleic acid-guided nuclease of the disclosure, an effector protein, a donor polynucleotide, a reporter element, a genetic element of interest, a component of a split system and/or any nucleic acid or proteinaceous molecule necessary to carry out the embodiments of the methods of the disclosure in either prokaryotic or eukaryotic cells.

Non-limiting examples of suitable eukaryotic promoters (i.e. promoters functional in a eukaryotic cell) may include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-active promoter (CAG), murine stem cell virus promoter (MSCV), phosphoglycerate kinase-1 locus promoter (PGK) and mouse metallothionein-I. The promoter may be a fungi promoter. The promoter may be a plant promoter. A database of plant promoters may be found (e.g., PlantProm). The expression vector may also contain a ribosome binding site for translation initiation and a transcription terminator. The expression vector may also include appropriate sequences for amplifying expression. The expression vector may also include nucleotide sequences encoding non-native tags (e.g., 6×His tag (SEQ ID NO: 5), hemagglutinin tag, green fluorescent protein, etc.) that are fused to the Argonaute, thus resulting in a fusion protein.

In some embodiments, a nucleotide sequence or sequences encoding a guide nucleic acid of the disclosure, a nucleic acid-guided nuclease (e.g., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to an inducible promoter (e.g., heat shock promoter, tetracycline-regulated promoter, steroid-regulated promoter, metal-regulated promoter, estrogen receptor-regulated promoter, etc.). In some embodiments, a nucleotide sequence encoding a guide nucleic acid of the disclosure, a nucleic acid-guided nuclease of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be operably linked to a constitutive promoter (e.g., CMV promoter, UBC promoter). In some embodiments, the nucleotide sequence may be operably linked to a spatially restricted and/or temporally restricted promoter (e.g., a tissue specific promoter, a cell type specific promoter, etc.).

A nucleotide sequence or sequences encoding a guide nucleic acid of the disclosure, a nucleic acid-guided nuclease (e.g., Cas9 or Argonaute) of the disclosure, an effector protein, a donor polynucleotide, a multiplexed genetic targeting agent, a tandem fusion polypeptide, a reporter element, a genetic element of interest, a component of a split system and/or any nucleic acid or proteinaceous molecule necessary to carry out the embodiments of the methods of the disclosure may be packaged into or on the surface of biological compartments for delivery to cells. Biological compartments may include, but are not limited to, viruses (lentivirus, adenovirus), nanospheres, liposomes, quantum dots, nanoparticles, polyethylene glycol particles, hydrogels, and micelles.

Introduction of the complexes, polypeptides, and nucleic acids of the disclosure into cells may occur by viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

### Codon-Optimization

A polynucleotide disclosed herein encoding a nucleic acid-guided nuclease (e.g., Cas9 or Argonaute) may be codon-optimized. This type of optimization may entail the mutation of foreign-derived (e.g., recombinant) DNA to mimic the codon preferences of the intended host organism or cell while encoding the same protein. Thus, the codons may be changed, but the encoded protein remains unchanged. For example, if the intended target cell was a human cell, a human codon-optimized polynucleotide Cas9 could be used for producing a suitable Cas9. As another non-limiting example, if the intended host cell were a mouse cell, then a mouse codon-optimized polynucleotide encoding Cas9 could be a suitable Cas9. A polynucleotide encoding a CRISPR/Cas protein may be codon optimized for many host cells of interest. A polynucleotide encoding an Argonaute may be codon optimized for many host cells of interest. A host cell may be a cell from any organism (e.g. a bacterial cell, an archaeal cell, a cell of a single-cell eukaryotic organism, a plant cell, an algal cell, e.g., Botryococcus braunii, Chlamydomonas reinhardtii, Nannochloropsis gaditana, Chlorella pyrenoidosa, Sargassum patens C. Agardh, and the like, a fungal cell (e.g., a yeast cell), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), etc. Codon optimization may not be required. In some instances, codon optimization may be preferable.

### Delivery

Site-directed nucleases of the disclosure may be endogenously or recombinantly expressed within a cell. Site-directed nucleases may be encoded on a chromosome, extrachromosomally, or on a plasmid, synthetic chromosome, or artificial chromosome. Additionally or alternatively, an site-directed nucleases may be provided or delivered to the cell as a polypeptide or mRNA encoding the polypeptide. In such examples, polypeptide or mRNA may be delivered through standard mechanisms known in the art, such as through the use of cell permeable peptides, nanoparticles, viral particles, viral delivery systems, or other non-viral delivery systems.

Additionally or alternatively, guide nucleic acids disclosed herein may be provided by genetic or episomal DNA within a cell. Guide nucleic acids may be reverse transcribed from RNA or mRNA within a cell. Guide nucleic acids may be provided or delivered to a cell expressing a corresponding site-directed nuclease. Additionally or alternatively, guide nucleic acids may be provided or delivered concomitantly with a site-directed nuclease or sequentially. Guide nucleic acids may be chemically synthesized, assembled, or otherwise generated using standard DNA or RNA generation techniques known in the art. Additionally or alternatively, guide nucleic acids may be cleaved, released, or otherwise derived from genomic DNA, episomal DNA molecules, isolated nucleic acid molecules, or any other source of nucleic acid molecules.

### Small Molecule Inhibitors

In some embodiments, the therapeutic agent is a small-molecule inhibitor. The small molecule inhibitor may be free of a polynucleotide. The small-molecule inhibitor may be free of a peptide. In some embodiments, the small-molecule inhibitor binds directly to proteins or structures related to the expression of p16a to disrupt their functions. In general, small molecule inhibitors easily pass through a cell membrane and may not require additional modifications to assist its cellular uptake.

### Gene Targets

Provided herein are methods of editing a gene disclosed herein with a CRISPR/Cas system. Further provided herein are methods of contacting an RNA expressed from a gene disclosed herein with an antisense oligonucleotide, thereby altering the production of a protein encoded by the gene. Further provided herein are methods of editing a gene disclosed herein or modifying the expression of a gene disclosed herein. In some embodiments, editing the gene or modifying the expression of the gene comprises reducing the expression of the gene, reducing expression of a product of the gene (e.g. RNA, protein), reducing an activity of the product of the gene, or a combination thereof.

In some embodiments, the gene encodes a nuclear receptor. In some embodiments, the gene encodes a leucine zipper protein. In some embodiments, the gene encodes an opsin protein. In some embodiments, the gene encodes a G coupled protein receptor. In some embodiments, the gene is a tumor suppressor gene. In some embodiments, the gene encodes a protein that promotes cellular senescence. In some embodiments, the gene encodes a protein that promotes cellular apoptosis. In some embodiments, the gene encodes a protein that promotes cellular differentiation. In some embodiments, the gene encodes a protein that inhibits cellular proliferation. In some embodiments, the gene encodes a protein that inhibits cell survival.

In some embodiments, the gene is characterized by a sequence having a sequence identifier (SEQ ID NO) provided herein. In some embodiments, the gene is characterized by a sequence having homology to or being homologous to a sequence identifier (SEQ ID NO) provided herein. The terms “homologous,” “homology,” or “percent homology,” when used herein to describe to an amino acid sequence or a nucleic acid sequence, relative to a reference sequence, may be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences may be determined using the most recent version of BLAST, as of the filing date of this application.

Any one of the genes disclosed herein may be a human gene. The gene may encode a protein expressed by a blood cell. The gene may encode hemoglobin. The gene may encode a protein expressed on a cell of an eye in a human subject. By way of non-limiting example, the gene may encode a G protein coupled receptor (GPCR). The GPCR may be selected from a gene encoding an opsin protein (e.g., rhodopsin) or a transducing (e.g., GNAT1). Also by way of non-limiting example, the gene may encode a leucine zipper protein. The gene may be a neural retina-specific leucine zipper gene (Nrl) gene. The gene may encode a Nrl protein. The gene may comprise at least 10 consecutive nucleotides of SEQ ID NO.: 1 or SEQ ID NO.: 2. Also, by way of non-limiting example, the gene may encode a nuclear receptor. The gene may be a photoreceptor cell-specific nuclear receptor (PNR) gene. The gene may encode a PNR protein. PNR is also referred to as NR2E3 (nuclear receptor subfamily 2, group E, member 3). The gene may comprise at least 10 consecutive nucleotides of SEQ ID NO.: 3 or SEQ ID NO.: 4. The gene may be a Mertk gene. The gene may be other ocular genes including a retinoblastoma gene, an athonal7 gene, a Pax6 gene.

Provided herein are methods that comprise modifications of genes disclosed herein in cells disclosed herein. The gene may be a non-ocular gene and the cell may be a non-ocular cell. By way of non-limiting example, the gene may be UMOD, TMEM174, SLC22A8, SLC12A1, SLC34A1, SLC22A12, SLC22A2, MCCD1, AQP2, SLC7A13, KCNJ1, SLC22A6 or Pax3 and the cell may be a cell of a kidney. By way of non-limiting example, the gene may be PNLIPRP1, SYCN, PRSS1, CTRB2, CELA2A, CTRB1, CELA3A, CELA3B, CTRC, CPA1, PNLIP or CPB1 and the cell may be a cell of the pancreas. By way of non-limiting example, the gene may be GFAP, OPALIN, OLIG2, GRIN1, OMG, SLC17A7, C1orf61, CREG2, NEUROD6, ZDHHC22, VSTM2B or PMP2 and the cell may be a cell of the brain. By way of non-limiting example, the gene may encode an immune checkpoint inhibitor and the cell may be a T cell. By way of non-limiting example, the gene may encode PD-1 and the cell may be a T cell. The gene may encode PD-L1 or PD-L2, and the cell may be a tumor cell.

### Cells

Provided herein are methods of modifying a nucleic acid molecule expressed by a cell disclosed herein. Further provided herein are methods of modifying expression and/or activity of a nucleic acid molecule expressed by a cell disclosed herein. In some embodiments, the methods comprise modifying the nucleic acid molecule or expression/activity thereof, wherein the nucleic acid molecule is present in a cell in vivo. In some embodiments, the methods comprise modifying the nucleic acid molecule or expression/activity thereof, wherein the nucleic acid molecule is present in a cell in vitro. In some embodiments, the methods comprise modifying the nucleic acid molecule or expression/activity thereof, wherein the nucleic acid molecule is present in a cell ex vivo. In some embodiments, the methods comprise modifying the nucleic acid molecule or expression/activity thereof, wherein the nucleic acid molecule is present in a cell in situ.

In some embodiments, the cell is a retinal cell. In some embodiments, the cell is a photoreceptor cell. In some embodiments, the photoreceptor cell is a rod. In some embodiments, the photoreceptor cell is a cone. In some embodiments, the photoreceptor cell is a photosensitive retinal ganglion cell. In some embodiments, the cell is an optic nerve cell. In some embodiments, the cell is a ganglion cell. In some embodiments, the cell is an amacrine cell. In some embodiments, the cell is a retinal ganglion cell.

In some embodiments, the cell has been isolated from the subject to be treated. In some embodiments, the cell is a stem cell. In some embodiments, the cell is a cord blood stem cell. In some embodiments, the cell is a blood cell. In some embodiments, the cell is a hematopoietic stem cell. In some embodiments, the cell is a hematopoietic pluripotent cell. In some embodiments, the cell is a cancer cell. In some embodiments, the cell is an epithelial cell. In some embodiments, the cell is an intestinal cell. In some embodiments, the cell is a pluripotent cell. In some embodiments, the cell is a multipotent cell. In some embodiments, the cell is an induced pluripotent stem cell (iPSC). In some embodiments, the iPSC was derived from a nerve cell. In some embodiments, the iPSC was derived from a cell of the eye. In some embodiments, the cell was an iPSC that was differentiated into a retinal ganglion cell or a multipotent progenitor thereof.

### Pharmaceutical Compositions & Modes of Administration

Disclosed herein are pharmaceutical compositions for the treatment of retinal degenerative conditions, comprising therapeutic agents described herein that inhibit gene expression and protein activity.

In some embodiments, the pharmaceutical composition is a formulation for administration to the eye. In some embodiments, the formulation for administration to the eye comprises a thickening agent, surfactant, wetting agent, base ingredient, carrier, excipient or salt that makes it suitable for administration to the eye. In some embodiments, the formulation for administration to the eye has a pH, salt or tonicity that makes it suitable for administration to the eye. These aspects of formulations for administration to the eye are described herein. In some embodiments, the pharmaceutical composition is an ophthalmic preparation. The pharmaceutical composition may comprise a thickening agent in order to prolong contact time of the pharmaceutical composition and the eye. In some embodiments, the thickening agent is selected from polyvinyl alcohol, polyethylene glycol, methyl cellulose, carboxy methyl cellulose, and combinations thereof. In some embodiments, the thickening agent is filtered and sterilized.

The pharmaceutical compositions disclosed herein may comprise a pharmaceutically acceptable carrier, pharmaceutically acceptable excipient or pharmaceutically acceptable salt for the eye. Non-limiting examples of pharmaceutically acceptable carriers, pharmaceutically acceptable excipients and pharmaceutically acceptable salts for they eye, include hyaluronan, boric acid, calcium chloride, sodium perborate, phophonic acid, potassium chloride, magnesium chloride, sodium borate, sodium phosphate, and sodium chloride

The pharmaceutical compositions disclosed herein should be isotonic with lachrymal secretions. In some embodiments, the pharmaceutical composition has a tonicity from 0.5-2% NaCl. In some embodiments, the pharmaceutical composition comprises an isotonic vehicle. By way of non-limiting example, an isotonic vehicle may comprise boric acid or monobasic sodium phosphate.

In some embodiments, the pharmaceutical composition has a pH from about 3 to about 8. In some embodiments, the pharmaceutical composition has a pH from about 3 to about 7. In some embodiments, the pharmaceutical composition has a pH from about 4 to about 7. Pharmaceutical compositions outside this pH range may irritate the eye or form particulates in the eye when administered.

In some embodiments, the pharmaceutical compositions disclosed herein comprise a surfactant or wetting agent. Non-limiting examples of a surfactant employed in the pharmaceutical compositions disclosed herein are venzalkonium chloride, polysorbate 20, polysorbate 80, and dioctyl sodium sulpho succinate.

In some embodiments, the pharmaceutical compositions disclosed herein comprise a preservative that prevents microbial contamination after a container holding the pharmaceutical composition has been opened. In some embodiments, the preservative is selected from benzalkonium chloride, chlorobutanol, phenylmercuric acetate, chlorhexidine acetate, and phenylmercuric nitrate.

In some embodiments, the pharmaceutical composition (e.g., a lotion or ointment) comprises a base ingredient. The base ingredient may be selected from sodium chloride, sodium bicarbonate, boric acid, borax, zinc sulfate, a paraffin, and a wax or fatty substance. In some embodiments, the pharmaceutical composition is a lotion. In some embodiments, the lotion is provided to the subject (or a subject administering the lotion), as a powder or lyophilized product, that is reconstituted immediately before use.

Administering the pharmaceutical composition directly to the eye may avoid any undesirable off-target effects of the therapeutic agents in locations other than the eye. For example, administering the pharmaceutical composition intravenously or systemically may result in inhibiting gene expression in cells other than those of the eye, where inhibiting the gene may have deleterious effects.

In some embodiments, the pharmaceutical composition comprises a polynucleotide vector encoding any one of the nucleic acid molecules (e.g., shRNA, guide RNA, nuclease encoding polynucleotide) disclosed herein. In some embodiments, the polynucleotide vector is an expression vector. In some embodiments, the polynucleotide vector is a viral vector. In some embodiments, the pharmaceutical composition comprises a virus, wherein the virus delivers the vector and/or nucleic acid molecule to a cell of the subject. In some embodiments, the virus is a retrovirus. In some embodiments, the virus is a lentivirus. In some embodiments, the virus is an adeno-associated virus (AAV). In some embodiments, the AAV is selected from serotypes 1, 2, 5, 7, 8 and 9. In some embodiments, the AAV is AAV serotype 2. In some embodiments, the AAV is AAV serotype 8.

AAV may be particularly useful for the methods disclosed herein due to a minimal stimulation of the immune system and its ability to provide expression for years in non-dividing retinal cells. AAV may be capable of transducing multiple cell types within the retina. In some embodiments, the methods comprise intravitreal administration (e.g. injected in the vitreous humor of the eye) of AAV. In some embodiments, the methods comprise subretinal administration of AAV (e.g. injected underneath the retina).

In some embodiments, the methods and compositions disclosed herein comprise an exogenously regulatable promoter system in the AAV vector. By way of non-limiting example, the exogenously regulatable promoter system may be a tetracycline-inducible expression system.

Pharmaceutical compositions disclosed herein may further comprise one or more pharmaceutically acceptable salts, excipients or vehicles. Pharmaceutically acceptable salts, excipients, or vehicles for use in the present pharmaceutical compositions include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

Neutral buffered saline or saline mixed with serum albumin may be exemplary appropriate carriers. The pharmaceutical compositions may include antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics, or polyethylene glycol (PEG). Also by way of example, suitable tonicity enhancing agents include alkali metal halides (preferably sodium or potassium chloride), mannitol, sorbitol, and the like. Suitable preservatives include benzalkonium chloride, thimerosal, phenethyl alcohol, methylparaben, propylparaben, chlorhexidine, sorbic acid and the like. Hydrogen peroxide also may be used as preservative. Suitable cosolvents include glycerin, propylene glycol, and PEG. Suitable complexing agents include caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal, and the like. The buffers may be conventional buffers such as acetate, borate, citrate, phosphate, bicarbonate, or Tris-HCl. Acetate buffer may be about pH 4-5.5, and Tris buffer may be about pH 7-8.5. Additional pharmaceutical agents are set forth in Remington's Pharmaceutical Sciences, 18th Edition, A. R. Gennaro, ed., Mack Publishing Company, 1990.

The composition may be in liquid form or in a lyophilized or freeze-dried form and may include one or more lyoprotectants, excipients, surfactants, high molecular weight structural additives and/or bulking agents (see, for example, U.S. Pat. Nos. 6,685,940, 6,566,329, and 6,372,716). In one embodiment, a lyoprotectant is included, which is a non-reducing sugar such as sucrose, lactose or trehalose. The amount of lyoprotectant generally included is such that, upon reconstitution, the resulting formulation will be isotonic, although hypertonic or slightly hypotonic formulations also may be suitable. In addition, the amount of lyoprotectant should be sufficient to prevent an unacceptable amount of degradation and/or aggregation of the protein upon lyophilization. Exemplary lyoprotectant concentrations for sugars (e.g., sucrose, lactose, trehalose) in the pre-lyophilized formulation are from about 10 mM to about 400 mM. In another embodiment, a surfactant is included, such as for example, nonionic surfactants and ionic surfactants such as polysorbates (e.g., polysorbate 20, polysorbate 80); poloxamers (e.g., poloxamer 188); poly(ethylene glycol) phenyl ethers (e.g., Triton); sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl, myristyl-, or cetyl-betaine; lauroamidopropyl-, cocamidopropyl-, linoleamidopropyl-, myristamidopropyl-, palmidopropyl-, or isostearamidopropyl-betaine (e.g., lauroamidopropyl); myristamidopropyl-, palmidopropyl-, or isostearamidopropyl-dimethylamine; sodium methyl cocoyl-, or disodium methyl ofeyl-taurate; the MONAQUAT™ series (Mona Industries, Inc., Paterson, N.J.), polyethyl glycol, polypropyl glycol, and copolymers of ethylene and propylene glycol (e.g., Pluronics, PF68 etc). Exemplary amounts of surfactant that may be present in the pre-lyophilized formulation are from about 0.001-0.5%. High molecular weight structural additives (e.g., fillers, binders) may include for example, acacia, albumin, alginic acid, calcium phosphate (dibasic), cellulose, carboxymethylcellulose, carboxymethylcellulose sodium, hydroxyethylcellulose, hydroxypropylcellulose, hydroxypropylmethylcellulose, microcrystalline cellulose, dextran, dextrin, dextrates, sucrose, tylose, pregelatinized starch, calcium sulfate, amylose, glycine, bentonite, maltose, sorbitol, ethylcellulose, disodium hydrogen phosphate, disodium phosphate, disodium pyrosulfite, polyvinyl alcohol, gelatin, glucose, guar gum, liquid glucose, compressible sugar, magnesium aluminum silicate, maltodextrin, polyethylene oxide, polymethacrylates, povidone, sodium alginate, tragacanth microcrystalline cellulose, starch, and zein. Exemplary concentrations of high molecular weight structural additives are from 0.1% to 10% by weight. In other embodiments, a bulking agent (e.g., mannitol, glycine) may be included.

Compositions may be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, or intralesional routes. A parenteral formulation typically will be a sterile, pyrogen-free, isotonic aqueous solution, optionally containing pharmaceutically acceptable preservatives.

Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringers' dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present, such as, for example, anti-microbials, antioxidants, chelating agents, inert gases and the like. See generally, Remington's Pharmaceutical Science, 16th Ed., Mack Eds., 1980.

Compositions described herein may be formulated for controlled or sustained delivery in a manner that provides local concentration of the product (e.g., bolus, depot effect) and/or increased stability or half-life in a particular local environment. The compositions may comprise the formulation of polypeptides, nucleic acids, or vectors disclosed herein with particulate preparations of polymeric compounds such as polylactic acid, polyglycolic acid, etc., as well as agents such as a biodegradable matrix, injectable microspheres, microcapsular particles, microcapsules, bioerodible particles beads, liposomes, and implantable delivery devices that provide for the controlled or sustained release of the active agent which then may be delivered as a depot injection. Techniques for formulating such sustained- or controlled-delivery means are known and a variety of polymers have been developed and used for the controlled release and delivery of drugs. Such polymers are typically biodegradable and biocompatible. Polymer hydrogels, including those formed by complexation of enantiomeric polymer or polypeptide segments, and hydrogels with temperature or pH sensitive properties, may be desirable for providing drug depot effect because of the mild and aqueous conditions involved in trapping bioactive protein agents. See, for example, the description of controlled release porous polymeric microparticles for the delivery of pharmaceutical compositions in WO 93/15722.

Suitable materials for this purpose may include polylactides (see, e.g., U.S. Pat. No. 3,773,919), polymers of poly-(a-hydroxycarboxylic acids), such as poly-D-(−)-3-hydroxybutyric acid (EP 133,988A), copolymers of L-glutamic acid and gamma ethyl-L-glutamate (Sidman et al., Biopolymers, 22: 547-556 (1983)), poly(2-hydroxyethyl-methacrylate) (Langer et al., J. Biomed. Mater. Res., 15: 167-277 (1981), and Langer, Chem. Tech., 12: 98-105 (1982)), ethylene vinyl acetate, or poly-D(−)-3-hydroxybutyric acid. Other biodegradable polymers include poly(lactones), poly(acetals), poly(orthoesters), and poly(orthocarbonates). Sustained-release compositions also may include liposomes, which may be prepared by any of several methods known in the art (see, e.g., Eppstein et al., Proc. Natl. Acad. Sci. USA, 82: 3688-92 (1985)). The carrier itself, or its degradation products, should be nontoxic in the target tissue and should not further aggravate the condition. This may be determined by routine screening in animal models of the target disorder or, if such models are unavailable, in normal animals.

Formulations suitable for intramuscular, subcutaneous, peritumoral, or intravenous injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an active agent may be optionally formulated in aqueous solutions, preferably in physiologically compatible buffers such as Hank's solution, Ringer's solution, or physiological saline buffer.

Parenteral injections optionally involve bolus injection or continuous infusion. Formulations for injection are optionally presented in unit dosage form, e.g., in ampoules or in multi dose containers, with an added preservative. The pharmaceutical composition described herein can be in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an active agent in water soluble form. Additionally, suspensions are optionally prepared as appropriate oily injection suspensions.

Alternatively or additionally, the compositions may be administered locally via implantation into the affected area of a membrane, sponge, or other appropriate material on to which a therapeutic agent disclosed herein has been absorbed or encapsulated. Where an implantation device is used, the device may be implanted into any suitable tissue or organ, and delivery of the therapeutic agent, nucleic acid, or vector disclosed herein may be directly through the device via bolus, or via continuous administration, or via catheter using continuous infusion.

Certain formulations comprising a therapeutic agent disclosed herein may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders also may be employed.

Suitable and/or preferred pharmaceutical formulations may be determined in view of the present disclosure and general knowledge of formulation technology, depending upon the intended route of administration, delivery format, and desired dosage. Regardless of the manner of administration, an effective dose may be calculated according to patient body weight, body surface area, or organ size.

Further refinement of the calculations for determining the appropriate dosage for treatment involving each of the formulations described herein are routinely made in the art and is within the ambit of tasks routinely performed in the art. Appropriate dosages may be ascertained through use of appropriate dose-response data.

“Pharmaceutically acceptable” may refer to approved or approvable by a regulatory agency of the Federal or a state government or listed in the U.S. Pharmacopeia or other generally recognized pharmacopeia for use in animals, including humans.

“Pharmaceutically acceptable salt” may refer to a salt of a compound that is pharmaceutically acceptable and that possesses the desired pharmacological activity of the parent compound.

“Pharmaceutically acceptable excipient, carrier or adjuvant” may refer to an excipient, carrier or adjuvant that may be administered to a subject, together with at least one antibody of the present disclosure, and which does not destroy the pharmacological activity thereof and is nontoxic when administered in doses sufficient to deliver a therapeutic amount of the compound.

“Pharmaceutically acceptable vehicle” may refer to a diluent, adjuvant, excipient, or carrier with which at least one antibody of the present disclosure is administered.

In some embodiments, the pharmaceutical composition is formulated for injectable administration. In some embodiments, the methods comprise injecting the pharmaceutical composition. In some embodiments, the methods comprise administering the pharmaceutical composition in a liquid form via intraocular injection. In some embodiments, the methods comprise administering the pharmaceutical composition in a liquid form via periocular injection. In some embodiments, the methods comprise administering the pharmaceutical composition in a liquid form via intravitreal injection. While some of these modes of administration may not be appealing to the subject (e.g. intravitreal injection), they may be most effective at penetrating barriers of the eye, and the therapeutic agent may be least likely to be washed away by tears or blinking as compared to eye drops, which offer convenience and low affordability.

In some embodiments, the methods comprise administering the pharmaceutical formulation systemically. In some embodiments, the therapeutic agent is a polynucleotide vector, wherein the polynucleotide vector comprises a guide RNA, antisense oligonucleotide or Cas encoding polynucleotide. The polynucleotide vector may comprise a conditional promoter for driving expression of the nucleic acid molecules of the vector in cell-specific manner. By way of non-limiting example, the conditional promoter may drive expression only in retinal ganglion cells or only drive expression to levels that have a functional effect in retinal ganglion cells.

In some embodiments, the pharmaceutical composition is formulated for non-injectable administration. In some embodiments, the pharmaceutical composition is formulated for topical administration. By way, of non-limiting example, the nucleic acid molecule may be suspended in a saline solution or buffer that is suitable for dropping into the eye

In some embodiments, the pharmaceutical composition may be formulated as an eye drop, a gel, a lotion, an ointment, a suspension or an emulsion. In some embodiments, the pharmaceutical composition is formulated in a solid preparation such as an ocular insert. For example, the ocular insert may be formed or shaped similar to a contact lens that releases the pharmaceutical composition over a period of time, effectively conveying an extended release formulation. The gel or ointment may be applied under or inside an eyelid or in a corner of the eye.

In some embodiments, the methods may comprise administering the pharmaceutical composition immediately before sleep or before a period of time in which the subject may maintain eye closure. In some embodiments, the methods comprise instructing the subject to keep their eyes closed or administering a cover (e.g., bandage, tape, patch) to maintain eye closure for at least 1 minute, at least 5 minutes, at least 10 minutes, at least 15 minutes, at least 20 minutes, at least 30 minutes, at least 1 hour, at least 2 hours, at least 4 hours, or at least 8 hours after the pharmaceutical composition is administered. The methods may comprise instructing the subject to keep their eyes closed from 1 minute to 8 hours after the pharmaceutical composition is administered. The methods may comprise instructing the subject to keep their eyes closed from 1 minute to 2 hours after the pharmaceutical composition is administered. The methods may comprise instructing the subject to keep their eyes closed from 1 minute to 30 minutes after the pharmaceutical composition is administered.

In some embodiments, the methods comprise administering the pharmaceutical composition to the subject only once to treat glaucoma. In some embodiments, the methods comprise administering the pharmaceutical composition a first time and a second time to treat glaucoma. The first time and the second time may be separated by a period of time ranging from one hour to twelve hours. The first time and the second time may be separated by a period of time ranging from one day to one week. The first time and the second time may be separated by a period of time ranging from one week to one month. In some embodiments, the methods comprise administering the pharmaceutical composition to the subject daily, weekly, monthly, or annually. In some embodiments, the methods may comprise an aggressive treatment initially, tapering to a maintenance treatment. By way of non-limiting example, the methods may comprise initially injecting the pharmaceutical composition followed by maintaining the treatment with the pharmaceutical composition administered in the form of eye drops. Also, by way of non-limiting example, the methods may comprise initially administering weekly injections of the pharmaceutical composition from about 1 week to about 20 weeks, followed by administering the pharmaceutical composition via injection or topical administration every two to twelve months.

In some embodiments, the therapeutic agent is a small molecule inhibitor, and the pharmaceutical composition is formulated for oral administration.

### Kits/Systems

Provided herein are kits and systems comprising a Cas nuclease or a polynucleotide encoding the Cas nuclease, a first guide RNA and a second guide RNA. The Cas nuclease and first/second guide RNAs may be any one of those disclosed herein. The first guide RNA may target Cas9 cleavage of a first site 5′ of at least a first region of a gene and the second guide RNA may target Cas9 cleavage of a second site 3′ of the first region of the gene, thereby excising the region of the gene, referred to as the excised region henceforth. The region may comprise an exon. The region may comprise a portion of an exon. The region may comprise about 1% to about 100% of the exon. The region may comprise about 2% to about 100% of the exon. The region may comprise about 5% to about 100% of the exon. The region may comprise about 5% to about 99% of the exon. The region may comprise about 1% to about 90% of the exon. The region may comprise about 5% to about 90% of the exon. The region may comprise about 10% to about 100% of the exon. The region may comprise about 10% to about 90% of the exon. The region may comprise about 15% to about 100% of the exon. The region may comprise about 15% to about 85% of the exon. The region may comprise about 20% to about 80% of the exon. The region may consist essentially of an exon. The region may comprise more than one exon. The region may comprise an intron or a portion thereof. The portion of the exon or intron may be at least about 1 nucleotide. The portion of the exon or intron may be at least about 5 nucleotide. The portion of the exon or intron may be at least about 10 nucleotides.

Provided herein are kits and systems comprising a donor polynucleotide disclosed herein. The donor polynucleotide may be comprise ends compatible with being inserted between the first site and the second site. The donor polynucleotide may be a donor exon comprising splice sites at the 5′ end and the 3′ end of the donor exon. The donor polynucleotide may comprise a donor exon comprising splice sites at the 5′ end and the 3′ end of the donor exon. The splice sites allow for inclusion of the exon in the open reading frame of the gene and thus, the splice sites would ensure the donor exon was transcribed in a cell of interest. The donor polynucleotide may comprise a wildtype sequence. The donor polynucleotide may be homologous to the excised region. The donor polynucleotide may be at least about 99% homologous to the excised region. The donor polynucleotide may be at least about 95% homologous to the excised region. The donor polynucleotide may be at least about 90% homologous to the excised region. The donor polynucleotide may be at least about 85% homologous to the excised region. The donor polynucleotide may be at least about 80% homologous to the excised region. The donor polynucleotide may be identical to the excised region except for the donor polynucleotide comprises a wildtype sequence where the excised region comprised a mutation. In some instances, the donor polynucleotide is not similar to the excised region. The donor polynucleotide may be less than about 90% homologous to the excised region. The donor polynucleotide may be less than about 80% homologous to the excised region. The donor polynucleotide may be less than about 70% homologous to the excised region. The donor polynucleotide may be less than about 60% homologous to the excised region. The donor polynucleotide may be less than about 50% homologous to the excised region. The donor polynucleotide may be less than about 40% homologous to the excised region. The donor polynucleotide may be less than about 30% homologous to the excised region. The donor polynucleotide may be less than about 20% homologous to the excised region. The donor polynucleotide may be less than about 10% homologous to the excised region. The donor polynucleotide may be less than about 8% homologous to the excised region. The donor polynucleotide may be less than about 5% homologous to the excised region. The donor polynucleotide may be less than about 2% homologous to the excised region.

Provided herein are kits and systems for treating an eye condition, comprising at least one guide RNA targeting a sequence in a gene selected from NRL and NR2E3. The first guide RNA and/or the second guide RNA may targets the Cas9 protein to a sequence comprising any one of SEQ ID NOS.: 1-4. The first guide RNA and/or the second guide RNA may targets the Cas9 protein to a sequence at least 90% homologous to any one of SEQ ID NOS.: 1-4.

### Certain Terminologies

Unless defined otherwise, all technical and scientific terms used herein have the same meaning as is commonly understood by one of skill in the art to which the claimed subject matter belongs. It is to be understood that the foregoing general description and the following examples are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. For example, “about 5 μL” means “about 5 μL” and also “5 μL.” Generally, the term “about” includes an amount that would be expected to be within experimental error. The term “about” includes values that are within 10% less to 10% greater of the value provided. For example, “about 50%” means “between 45% and 55%.” Also, by way of example, “about 30” means “between 27 and 33.”

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value. A p-value of less than 0.05 is considered statistically significant.

As used herein, the term “treating” and “treatment” refers to administering to a subject an effective amount of a composition so that the subject as a reduction in at least one symptom of the disease or an improvement in the disease, for example, beneficial or desired clinical results. For purposes of this invention, beneficial or desired clinical results include, but are not limited to, alleviation of one or more symptoms, diminishment of extent of disease, stabilized (e.g., not worsening) state of disease, delay or slowing of disease progression, amelioration or palliation of the disease state, and remission (whether partial or total), whether detectable or undetectable. Alternatively, treatment is “effective” if the progression of a disease is reduced or halted. Those in need of treatment include those already diagnosed with a disease or condition, as well as those likely to develop a disease or condition due to genetic susceptibility or other factors which contribute to the disease or condition, such as a non-limiting example, weight, diet and health of a subject are factors which may contribute to a subject likely to develop diabetes mellitus. Those in need of treatment also include subjects in need of medical or surgical attention, care, or management.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The examples and embodiments described herein are for illustrative purposes only and are not intended to limit the scope of the claims provided herein. Various modifications or changes suggested to persons skilled in the art are to be included within the spirit and purview of this application and scope of the appended claims.

### Example 1. CRISPR-Cas9 Targeting with Two Guide RNAs In Vitro

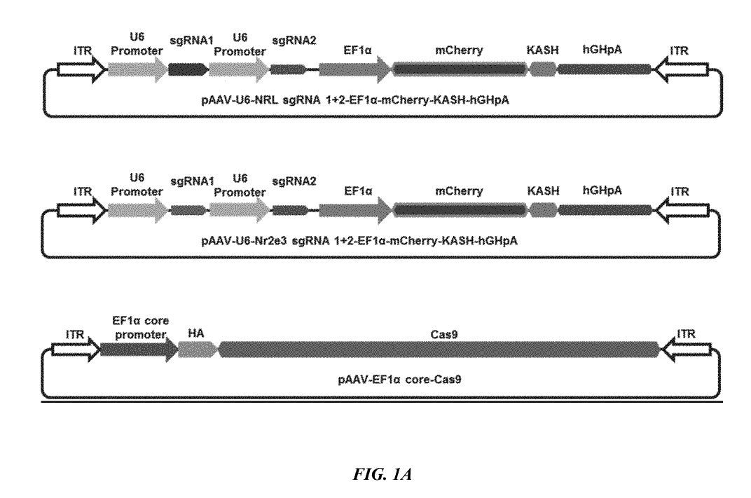

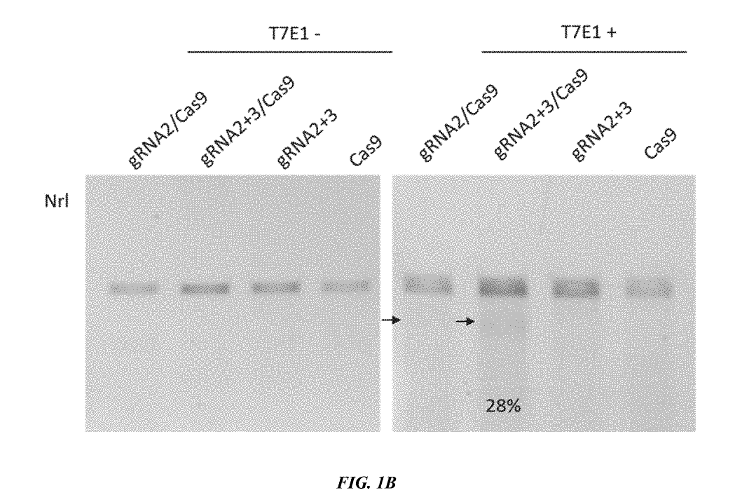

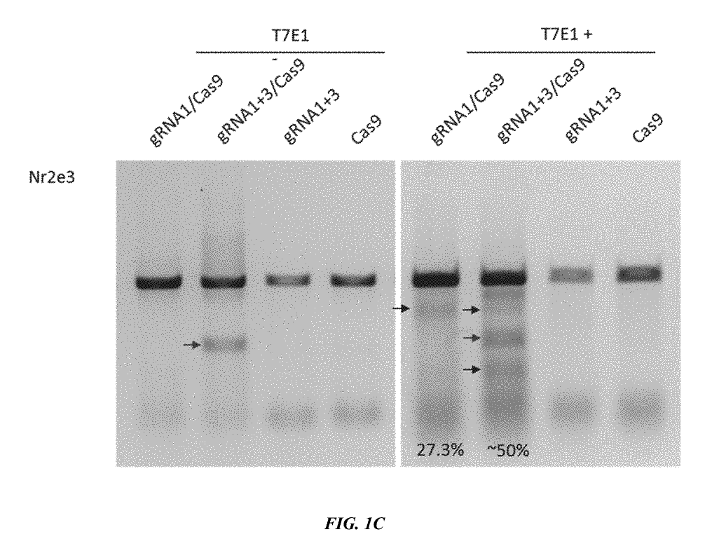

To test a CRISPR-CAS9 based cellular reprogramming strategy to treat RP and preserve visual function, two AAV vectors were employed, one expressing Cas9, and another carrying gRNAs targeting NRL or NR2E3 gene (see FIG. 1A). To construct double gRNA expression vectors, pAAV-U6 gRNA-EF1a mCherry was used. Both 20 bp gRNA sequences were sub-cloned into the vector separately. The CRISPR/Cas9 target sequences (20 bp target and 3 bp PAM sequence showed with underline) used in this study are shown as following: GAGCCTTCTGAGGGCCGATC TGG (SEQ ID NO. 1), and GTATGGTGTGGAGCCCAACG AGG (SEQ ID NO. 2) for NRL knockdown, GGCCTGGCACTGATTGCGAT GGG (SEQ ID NO. 3), and AGGCCTGGCACTGATTGCGA TGG (SEQ ID NO. 4) for NR2E3 knockdown. Targeting and inactivation efficiency by simultaneously targeting two sites by two gRNAs in the same gene was assessed against targeting and inactivation efficiency of a single gRNA. Gene knockdown efficiency in mouse fibroblasts was tested using a T7E1 nuclease assay which cleaves a mismatched double stranded DNA template. The knockdown efficiency of the two-gRNA system had much higher editing efficiency than that by a single-guided RNA system (see FIGS. 1B and 1C). Consequently, the double targeting knockout strategy was adopted in all subsequent in vivo experiments.

### Example 2. CRISPR-Cas9 Targeting with Two Guide RNAs In Vivo

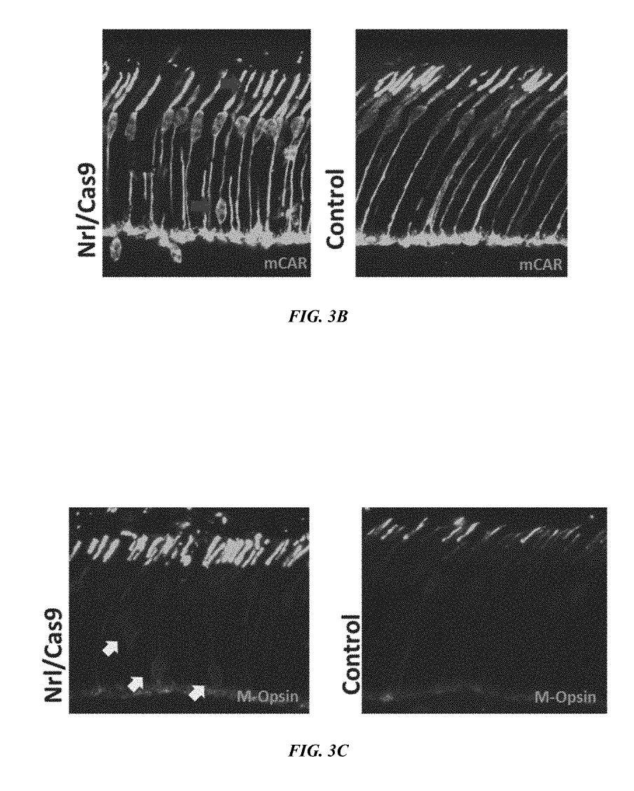

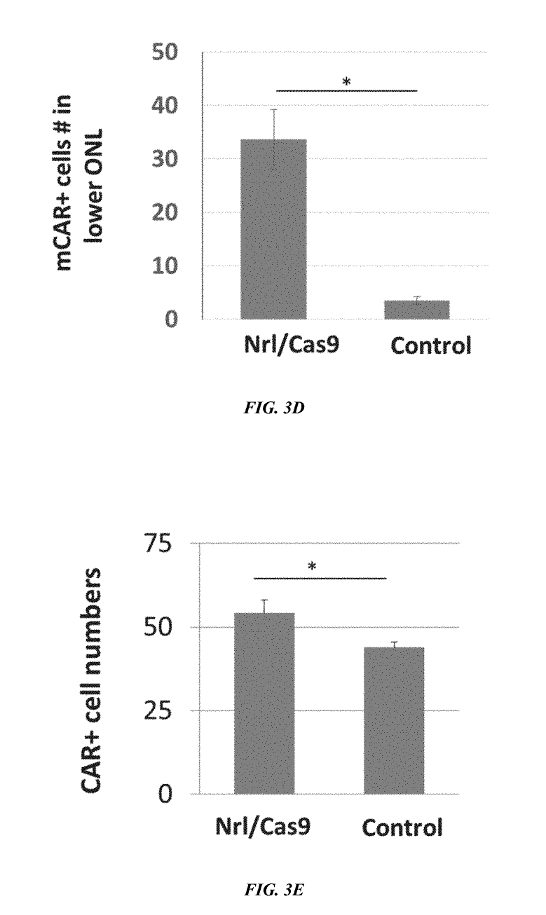

AAVs encoding Cas 9 and two guide RNAs targeting the NRL gene were delivered to WT mice via subretinal injections at P0 (postnatal day 7). Briefly, eyes of anesthetized mice were dilated and, under direct visualization with a dissecting microscope, 1 μl AAV mixture was injected into the subretinal space through a small incision using a glass micropipette (internal diameter 50˜75 μm) and a pump microinjection apparatus (Picospritzer III; Parker Hannifin Corporation). Successful injections were noted by creation of a small subretinal fluid bleb. Any mice showing retinal damage, such as bleeding, were not included in the study. P30 mice were sacrificed for histology. Retinas were frozen sectioned and stained for cone markers, including anti-mouse cone arrestin (mCAR) antibody and anti-medium wavelength opsin (M-opsin) antibody. mCherry was also imaged as a marker to label transduced areas and cells by AAV vectors. Results showed that AAV8-Cas9+AAV8-NRL gRNA1-mCherry could not induce any phenotype, suggesting that a single gRNA1 was not able to introduce genomic sequence disruption efficiently. Consistent with the in vitro T7E1 assay, a fate switch phenotype was observed with two gRNA in vivo. In control retinas, cone nuclei reside at the top layer of ONL, while rod nuclei fill the rest of ONL (see FIG. 3A). Retinas transduced with AAV8-Cas9+AAV8-NRL gRNA2+3-mCherry were observed, and there were a number of mCAR+ cells in the lower outer nuclear layer (ONL) (see FIG. 3B). The extra mCAR+ cells at the lower ONL layers have normal rod outer segment (see FIG. 3B). Extra mCAR+ cells at the lower ONL layers were not observed in the left uninjected control retinas. Quantification shows that there was significant increase of extra mCAR+ cells at the lower ONL layers in the AAV8-Cas9+AAV8-NRL gRNA2+3-mCherry coinjected group (FIG. 3D). Staining with M-opsin antibody also showed that these cells express another cone-specific gene, Opn1mw (FIG. 3C), suggesting the feasibility of a cone-like gene expression program.

### Example 3. Subretinal Injections of Retinal Pigmentosa (RP) Model Mouse with AAV Encoding Cas9/CRISPR System Targeting NRL or NR2E3

To test the hypothesis that partial conversion of degenerating rods into cones is sufficient to rescue retinal degeneration and restore retinal function, AAV-gRNA/Cas9 was injected into the subretinal space in RD10 mice at P0. RD10 mice are a model of autosomal recessive RP in humans with rapid rod photoreceptor degeneration. RD10 mice carry a spontaneous mutation of the rod-phosphodiesterase (PDE) gene, leading to rapid rod degeneration that starts around P18. Rod degeneration completes in postnatal 60 days with concurrent cone degeneration. Because photoreceptor degeneration does not overlap with retinal development, and light responses can be recorded for about a month after birth, RD10 mice mimic typical human RP more closely than other RD models such as rd1 mutants.

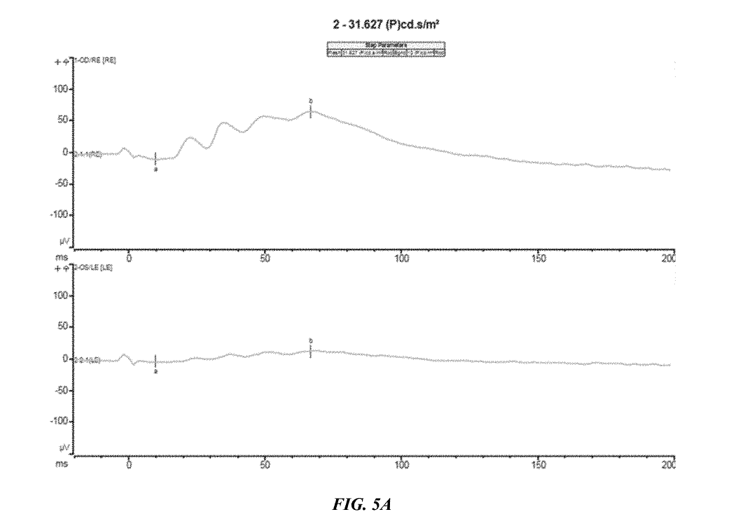

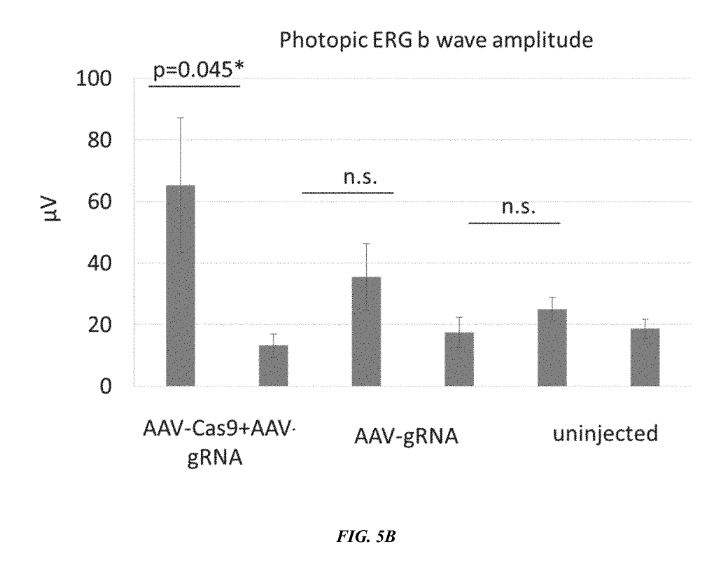

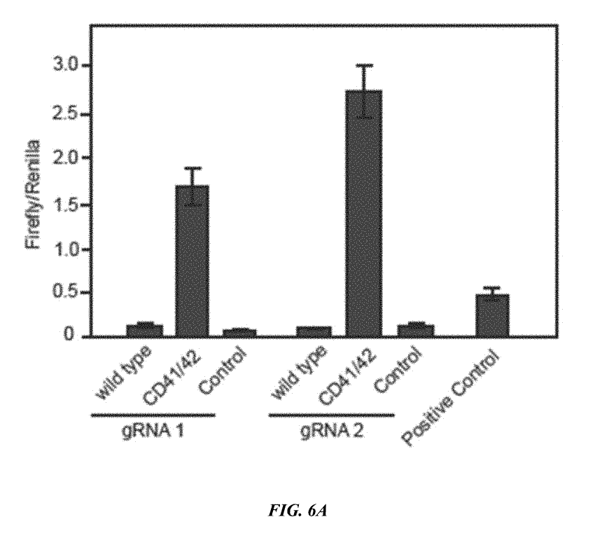

Analyses were performed between postnatal 7-8 weeks. To determine the effect of this AAV-gRNA/Cas9 treatment on the physiological function of the retina, electroretinography (ERG) responses were tested to measure the electrical activity of rods (scotopic, scotopic ERG was done but data not analyzed yet) and cones (photopic). The ERG tests were performed 6 weeks after the injection (P50). All eyes treated with AAV-gRNA/Cas9 exhibited significantly improved photopic b-wave value, suggesting enhanced cone function (see FIG. 5B). These results demonstrate that AAV-gRNA/Cas9 treatment rescued photoreceptor degeneration and preserve retinal visual function.

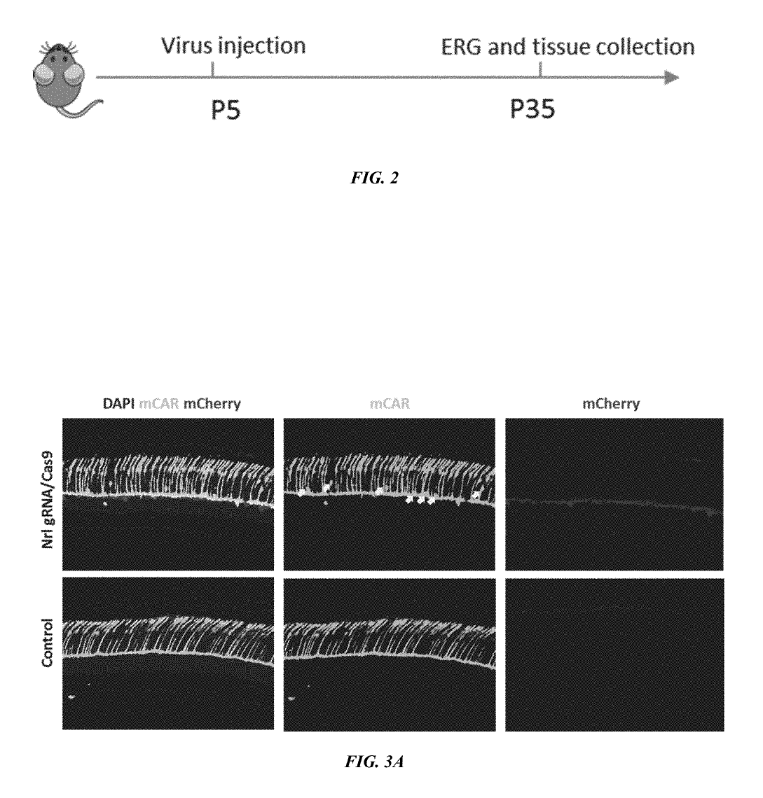

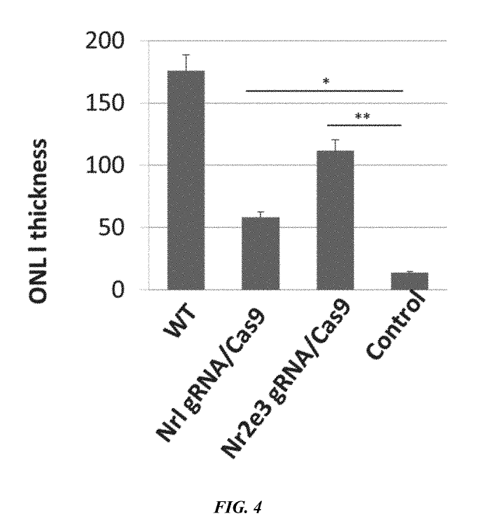

DNA analysis revealed correct knockdown in the AAV-gRNA/Cas9 injected eye (see FIG. 2C). In addition, AAV-gRNA/Cas9 injection led to significant improved preservation of the ONL thickness compared with that of non-injected controls (see FIG. 4C). Unlike untreated eyes which had only 1˜2 (or sparsely distributed) photoreceptor cell nuclei in the ONL, there were 3˜5 layers of ONL, indicating AAV-gRNA/Cas9 treatment prevented photoreceptor cell degeneration. Quantitative RT-PCR (qRT-PCR) was used to measure the relative expression levels of rod and cone photoreceptor genes (see FIG. 5C). These analyses showed an increased expression of cone specific genes.

Notably, a significant increase in ONL thickness was observed in treated eyes. Interestingly, many cells in ONL did not express either rods or cone markers, suggesting they may have been reprogrammed into an intermediate cell fate. One additional or alternative explanation of the observed rescue effect is that these intermediate cells down-regulate rod specific genes therefore rendering them resistant to death/degeneration caused by a rod specific gene mutation. These intermediate cells may have maintained a normal tissue structural integrity and secreted trophic factors essential for endogenous cone survival. Therefore visual function gain may have been partially due to a rescue effect in existing cones, rather than reprogramming of rods to cone fate.

### Example 4. Targeting Hemoglobin Gene Mutation with Cas-Mediated Homology Directed Repair for Treatment of Beta Thalassemia

Beta thalassemia is a blood disorder that reduces the production of hemoglobin (Hb). A mutation known as CD41/42 (-TCTT), in the Hb-encoding gene, is associated with this disorder. Repair of this gene may have therapeutic effects for subjects with this disorder.

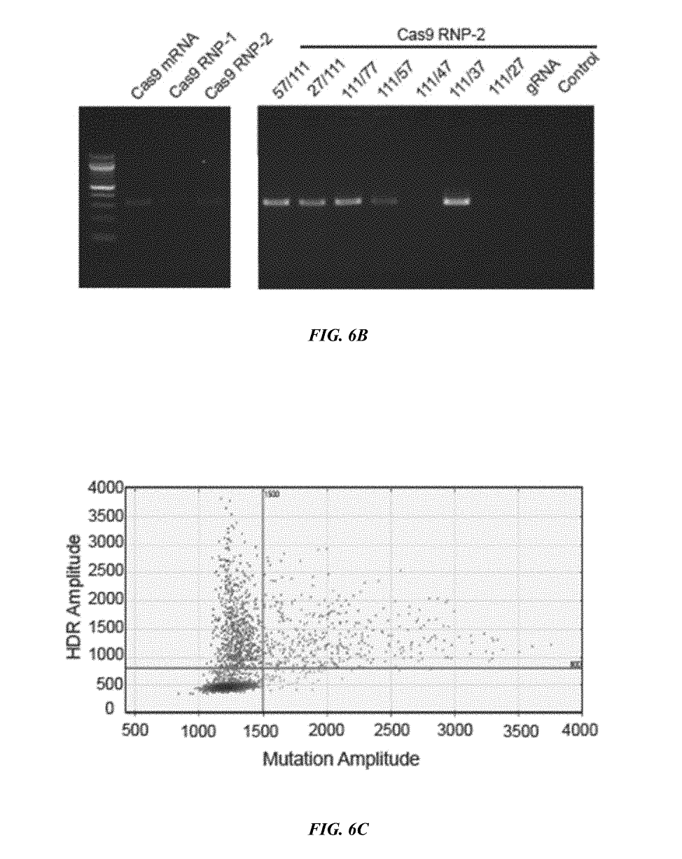

To specifically target both homogenous and heterogeneous CD41/42 mutation in patient-derived hematopoietic stem/progenitor cells (HSPC), two CRISPR/Cas9 target sequences that locate at mutation site were chosen. The specificity and efficiency were then tested using luciferase assay based on single strand annealing principle (SSA). SSA is a process that is initiated when a double strand bread is made between two repeated sequences oriented in the same direction. By putting wild type and CD41/42 mutation sequences between two partially-repeated luciferase expression cassettes, luciferase expression is activated when specific cutting is mediated by CRISPR/Cas9 system. Both gRNA-1 and gRNA-2 showed decent specificity, and gRNA-2 contained higher efficiency (FIG. 6A). gRNA-2 was chosen for further HSPC editing. Next, editing efficiencies of different Cas9 formats and single-stranded oligodeoxynucleotides (ssODNs) were tested. The HDR-mediated editing was assessed by both HDR specific PCR and droplet digital PCR. Among Cas9 mRNA and two Cas9 RNPs, Cas9 RNP-2 showed highest HDR efficiency (FIG. 6B, Left). Seven asymmetric ssODNs were designed and screened using Cas9 RNP-2, of which ssODN-111/37 scored highest HSPC editing efficiency (FIG. 6B, Left and 6C).

Plasmids.

To construct gRNA expression vectors, pX330(Addgene, 42230) was used. Two mutation-specific target sequences were sub-cloned into the vector separately as described previously. The CRISPR/Cas9 target sequences (20 bp target and 3 bp PAM sequence showed with underline) used in this study are shown as following: gRNA-1: GGCTGCTGGTGGTCTACCCTTGG (SEQ ID NO.: 6); gRNA-2: GGTAGACCACCAGCAGCCTAAGG (SEQ ID NO.: 7). Plasmid for in-vitro transcription of Cas9 was purchased.

Luciferase Assay.

To select mutation specific gRNAs, wild-type and CD41/42 mutated sequences were synthesized and cloned into pGL4-SSA, separately. pX330-gRNA-Cas9, pGL4-SSA-HBB, and pGL4-hRluc were co-transfected into 293T cells. Luciferase assay was performed using dual-luciferase reporter assay system.

In-Vitro Transcription.

Template for in vitro transcription of gRNA-2 was amplified using primers: gRNA-2-F: TAATACGACTCACTATAGGGACCCAGAGGTTGAGTCCTT (SEQ ID NO.: 8) and gRNA-F: AAAAGCACCGACTCGGTGCC (SEQ ID NO.: 9); Plasmid MLM 3639 was linearized and then used for Cas9 in-vitro transcription. gRNA and Cas9 were in vitro transcribed, purified and used for HSPC electroporation.

Assembly of Cas9 RNP.

To electroporate a 20 μl cell suspension (100,000 cells) with Cas9 RNP, a 5 μl gRNA solution was prepared by adding 1.2 molar excess of gRNA in Cas9 buffer. Another 5 μl solution containing 100 pmol Cas9 was added to the gRNA solution slowly, and incubated at room temperature for >10 minutes prior to mixing with target cells.

Isolation and Culture of Patient Derived CD34+ HSPC.

Cryopreserved mobilized peripheral blood PBMC from patients with CD41/42 mutation were used for HSPC isolation and culture.

HBB editing in patient derived CD34+ HSPC.

To edit patient derived HSPCs, HSPCs were isolated and cultured as described previously two days prior to electroporation with Cas9 mRNA or Cas9 RNP. 100,000 HSPCs were pelleted and resuspended in 20 μl Lonza P3 solution, and mixed with 10 ul Cas9 RNP and 1 ul 100 uM ssODN template, or same molars of Cas9 mRNA, gRNA, and 1 ul 100 uM ssODN template. This mixture was electroporated, genotyped and used for erythroid differentiation.

Genotyping of Edited Cells.

HDR specific PCR was performed with a HDR-specific forward primer and a universal reverse primer, HDR-F: CCCAGAGGTTCTTCGAATCC (SEQ ID NO.: 10); Universal-R: TCATTCGTCTGTTTCCCATTC (SEQ ID NO.: 11). BstBI (NEB, R0519) restriction digestion was also used for assessing HDR-mediated editing: a region around CD41/42 mutation was amplified first and then digested with BstBI for Mutation to HDR edits. HDR-mediated editing of CD41/42 mutation was also assessed by droplet digital PCR (ddPCR, QX200, Bio-Rad Laboratories, Inc.) HBB-F: CTGCCTATTGGTCTATTTTCC (SEQ ID NO.: 12); HBB-R: ACTCAGTGTGGCAAAGGTG (SEQ ID NO.: 13); Probe-donor: 6-FAM/CCCAGAGGTTCTTCGAATCCTTTG/BHQ1 (SEQ ID NO.: 14); Probe-mutation: HEX/CTTGGACCC AGAGGTTGAGTCC/BHQ1 (SEQ ID NO.: 15).

Flow Cytometry.

HSPC after isolation and electroporation were analyzed on LSR cell analyzer (BD Biosciences) for purity and lineaging.

Targeted Deep Sequencing.

The top 12 predicted off-target sites were searched using The CRISPR Design Tool. The on-target and potential off-target regions were amplified using from the HSPC DNA and used for library construction. The primers to amplify genomic regions are listed as following: HBB-F: TCGTCGGCAGCGTCAGATGTGTATAAGAGACAGCTGCCTATTGGTCTATTTTCC (SEQ ID NO.: 16); HBB-R: GTCTCGTGGGCTCGGAGATGTGTATAAGAGACAGACTCAGTGTGGCAAAGGTG (SEQ ID NO.: 17). Next PCR amplicons from first step were purified using Ampure beads (Beckman Coulter), and then subject to second round PCR to attach sample-specific barcodes. The purified PCR products were pooled at equal ratio for pair-end sequencing using Illumina MiSeq. The raw reads were mapped to mouse reference genome mm9. High quality reads (score >30) were analyzed for insertion and deletion (indel) events and Maximum Likelihood Estimate (MILE) calculation as previously described. As next generation sequencing analysis of indels cannot detect large size deletion and insertion events, CRISPR-Cas9 targeting efficiency and activity shown above is underestimated.

### Example 5. Homology-Independent Targeted Integration (HITI) Gene Replacement Therapy for Retinal Deneration In Vivo

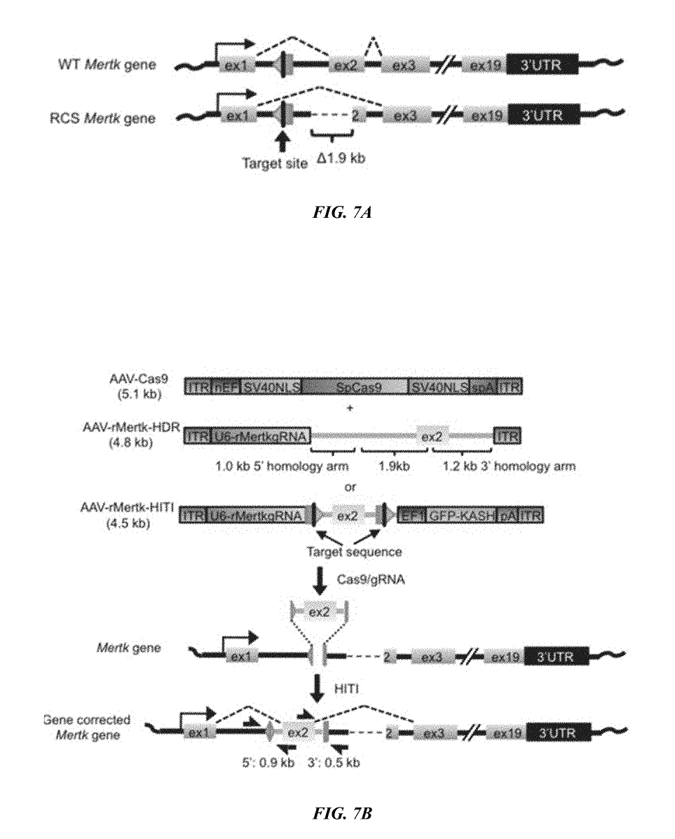

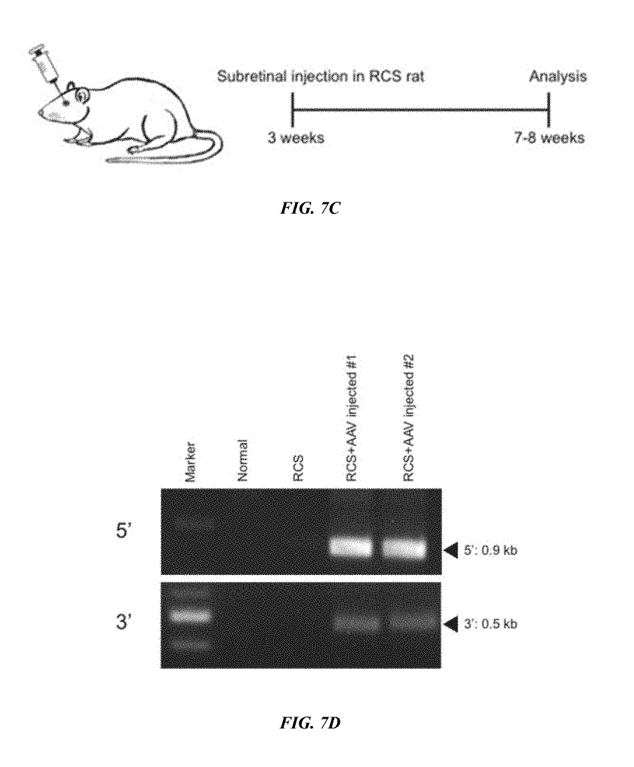

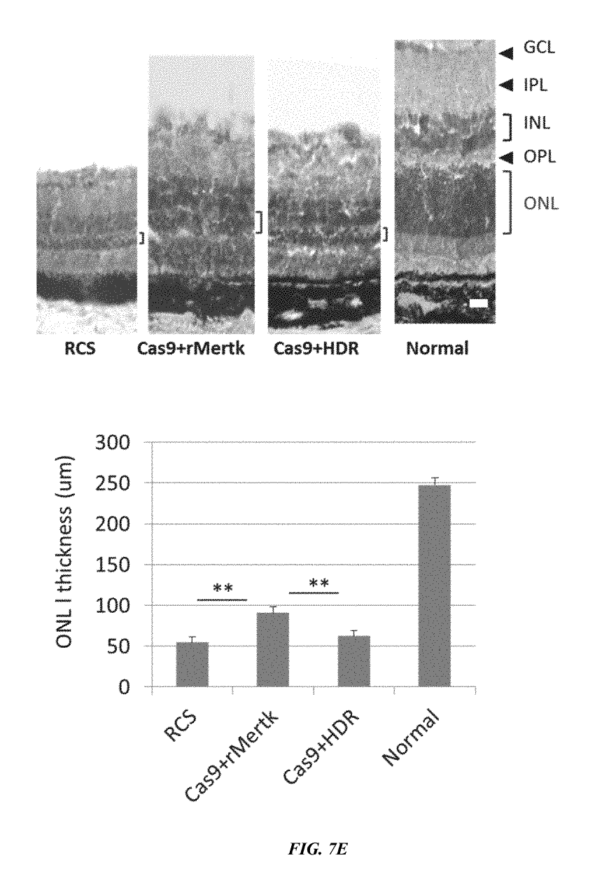

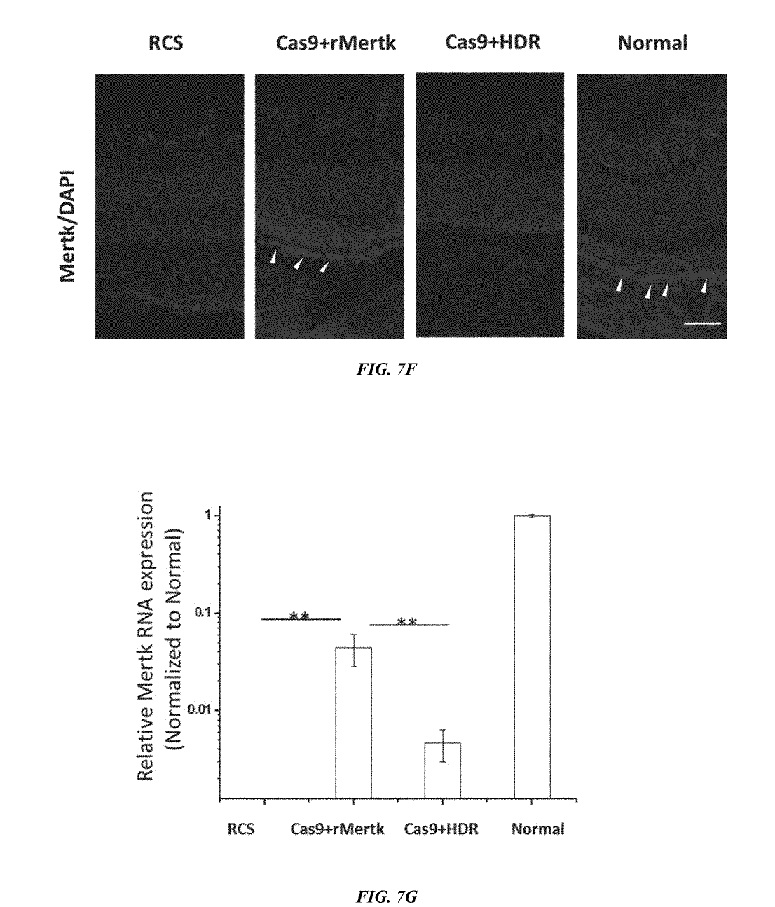

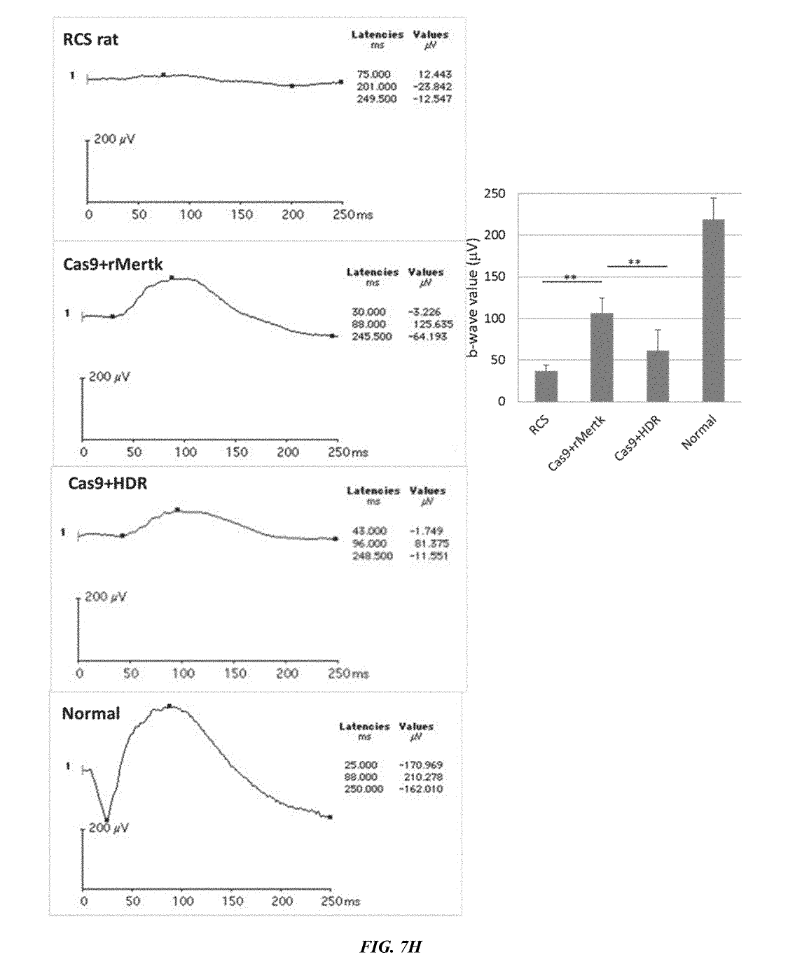

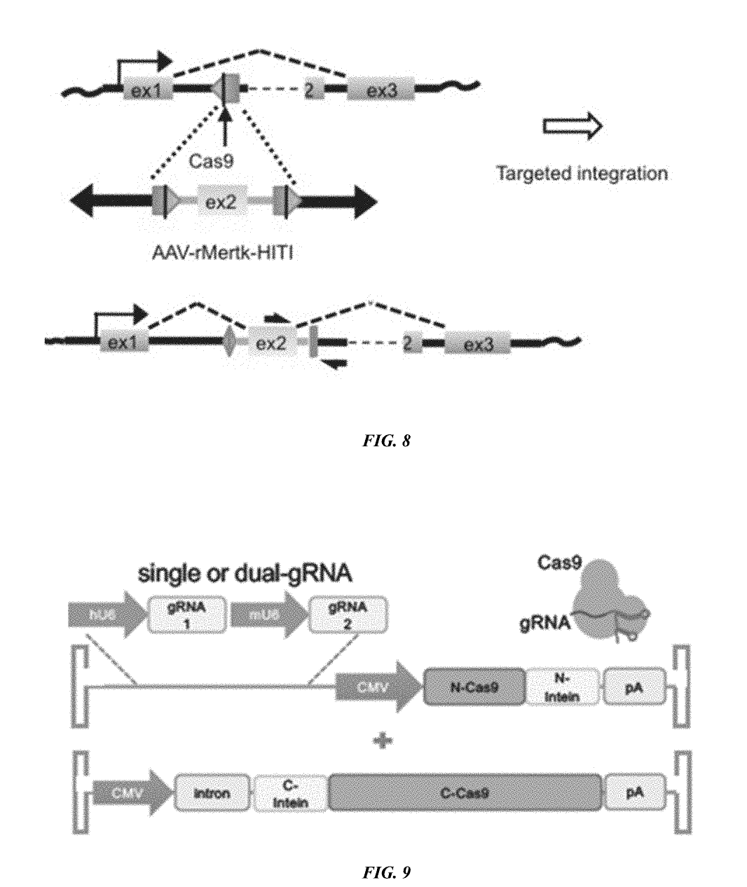

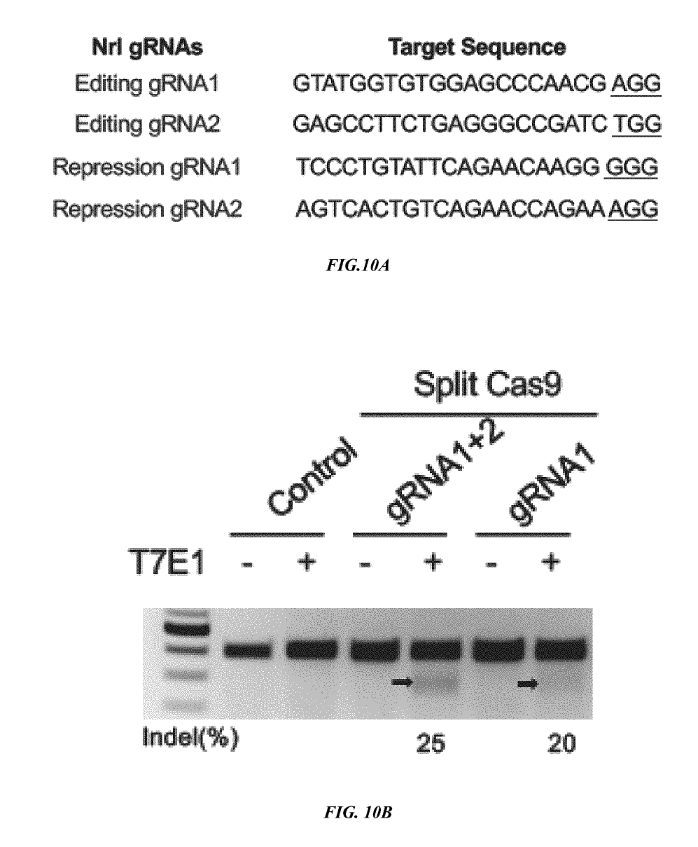

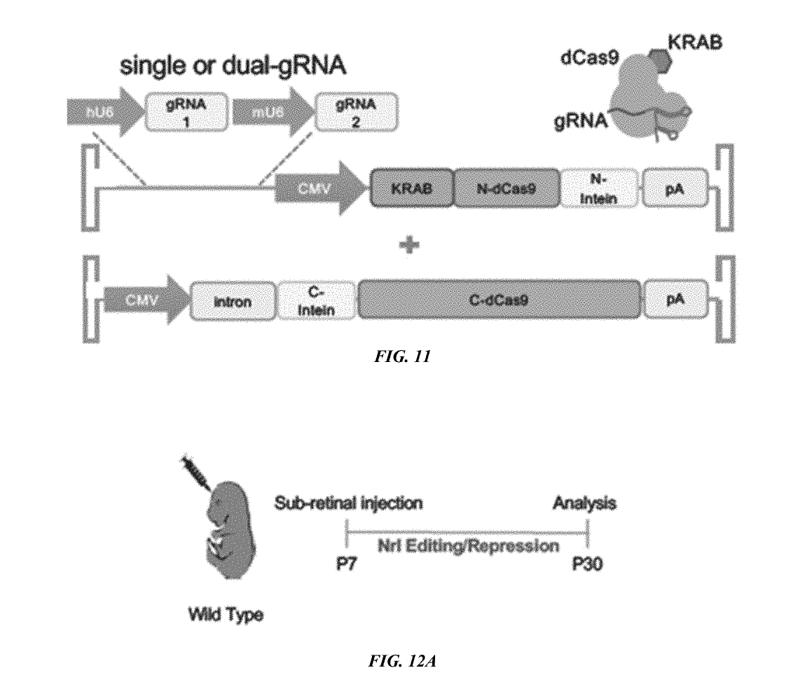

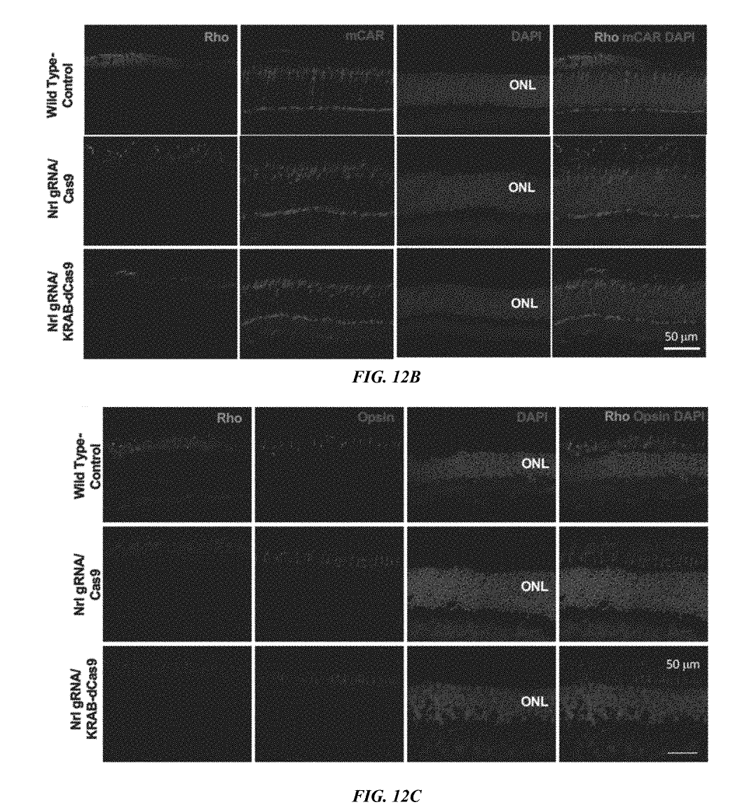

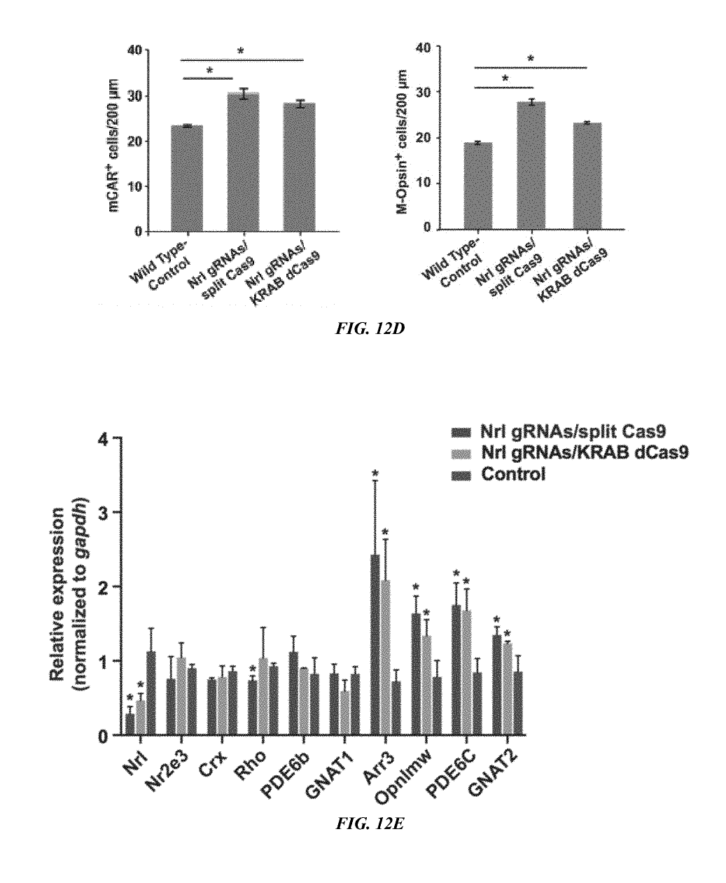

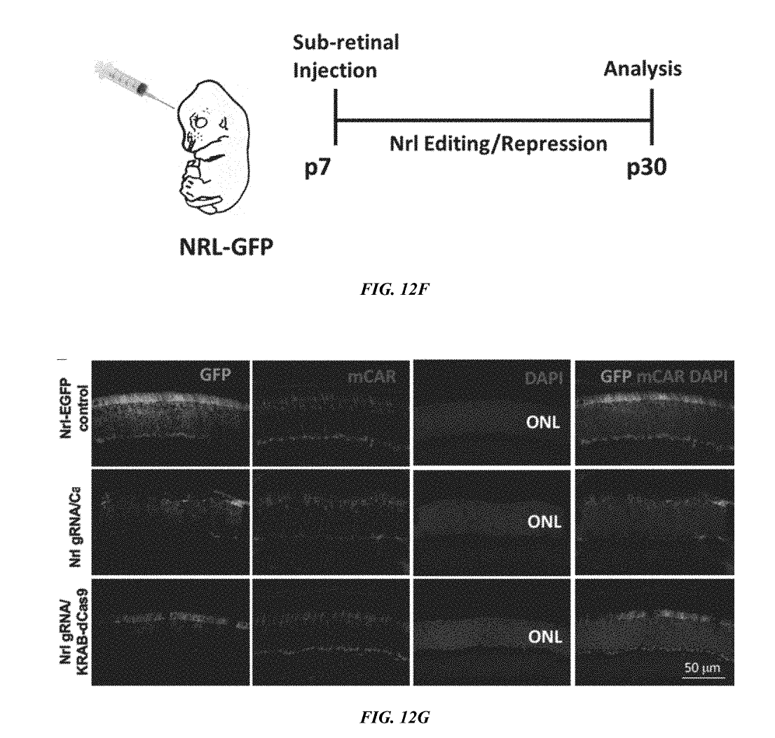

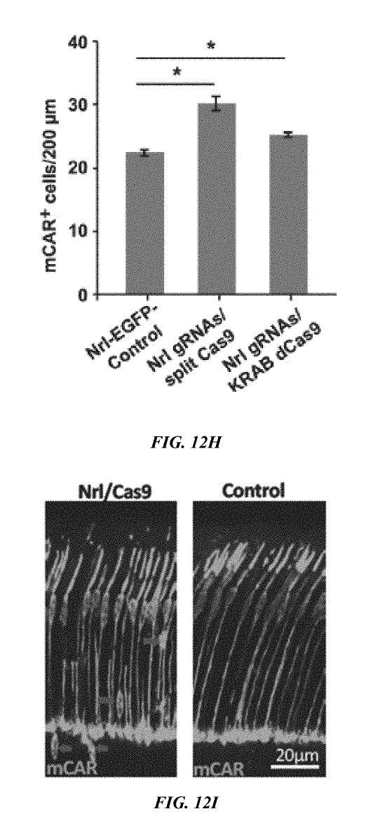

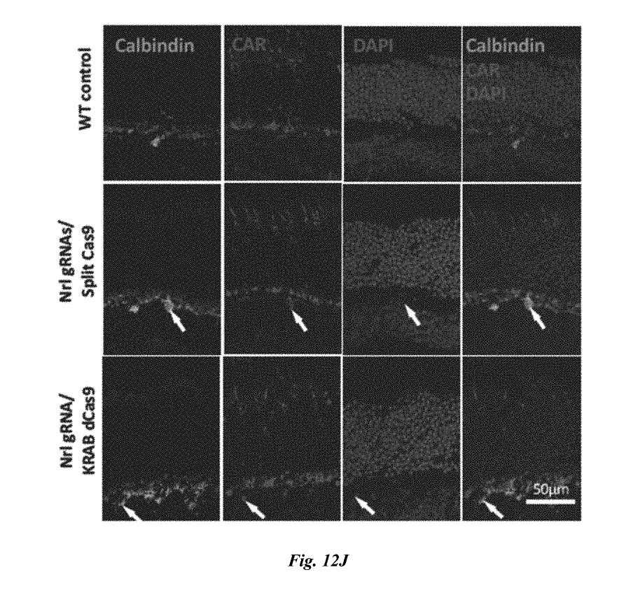

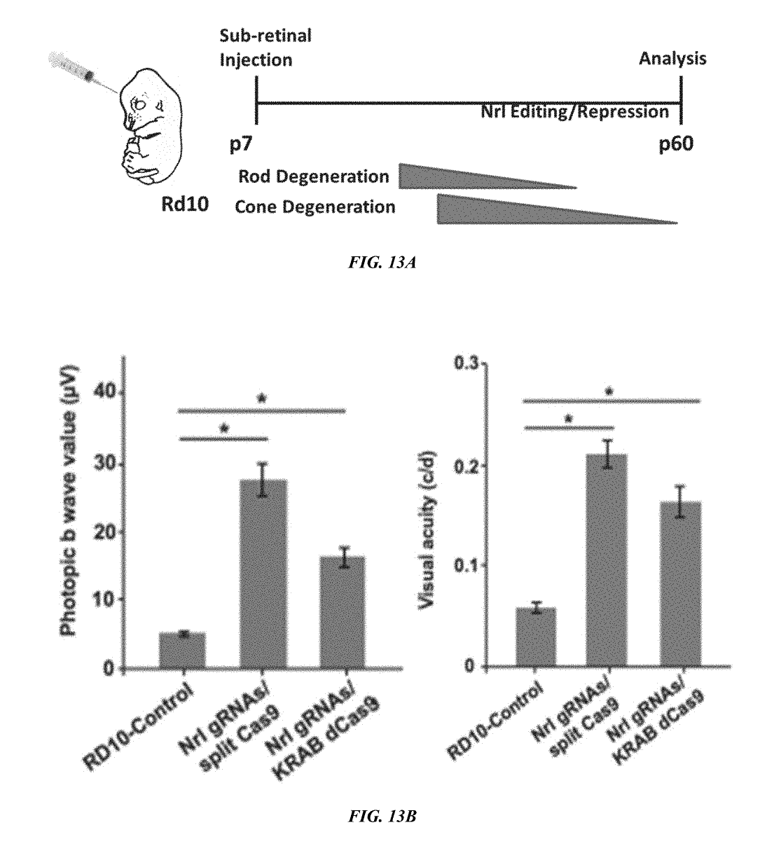

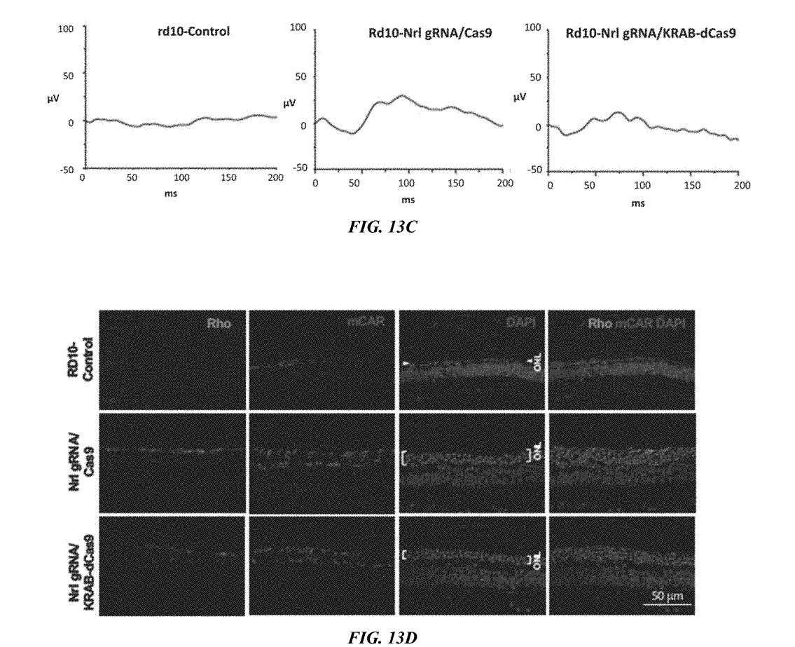

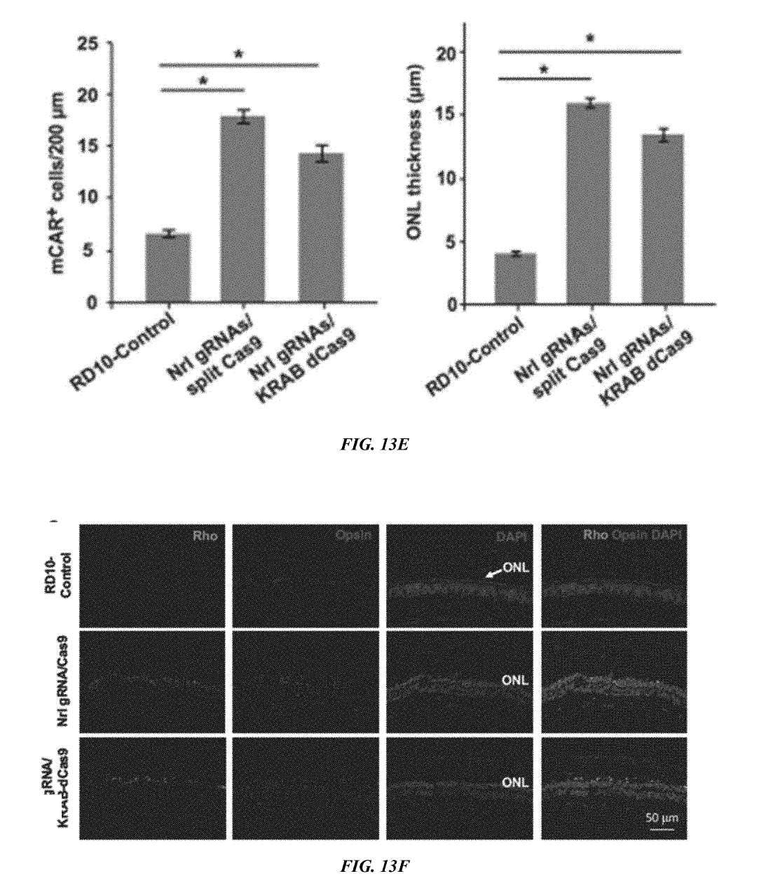

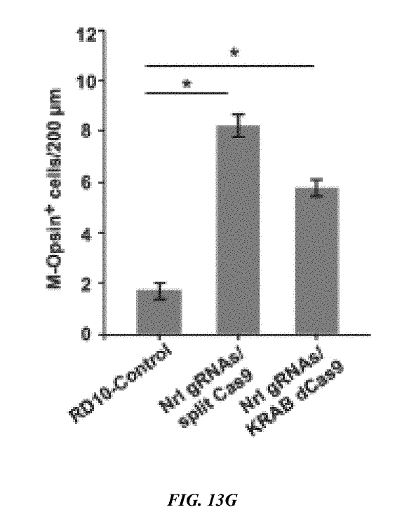

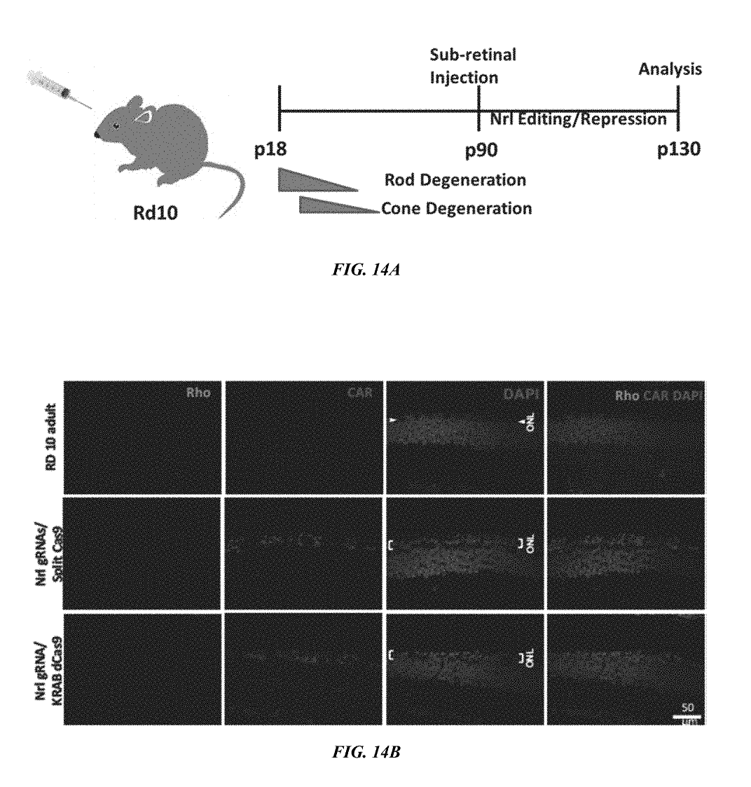

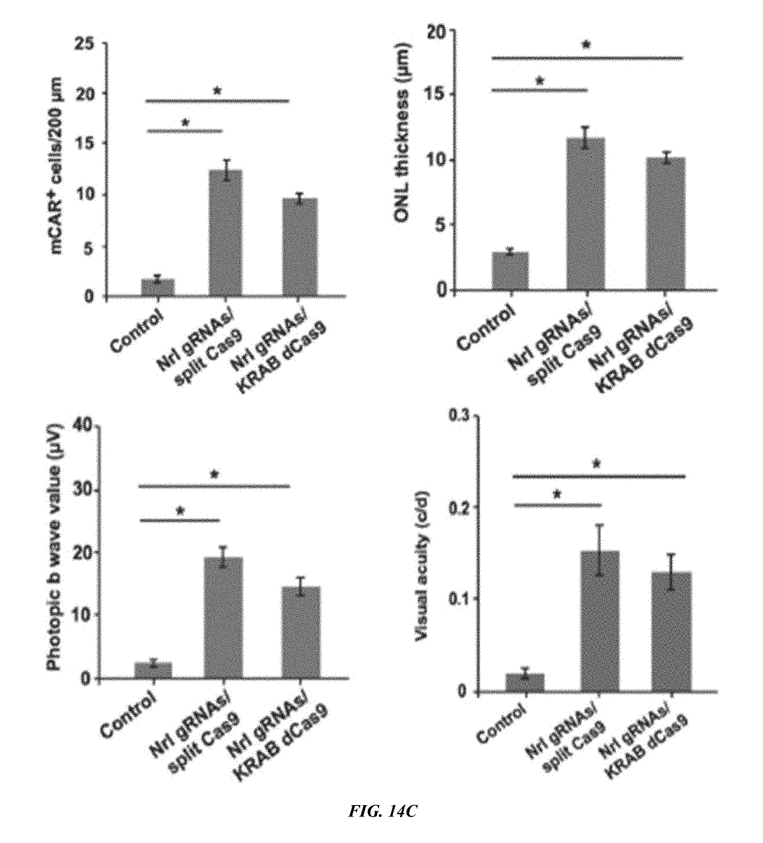

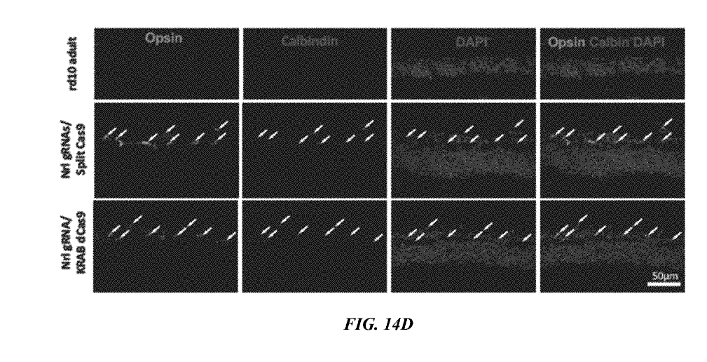

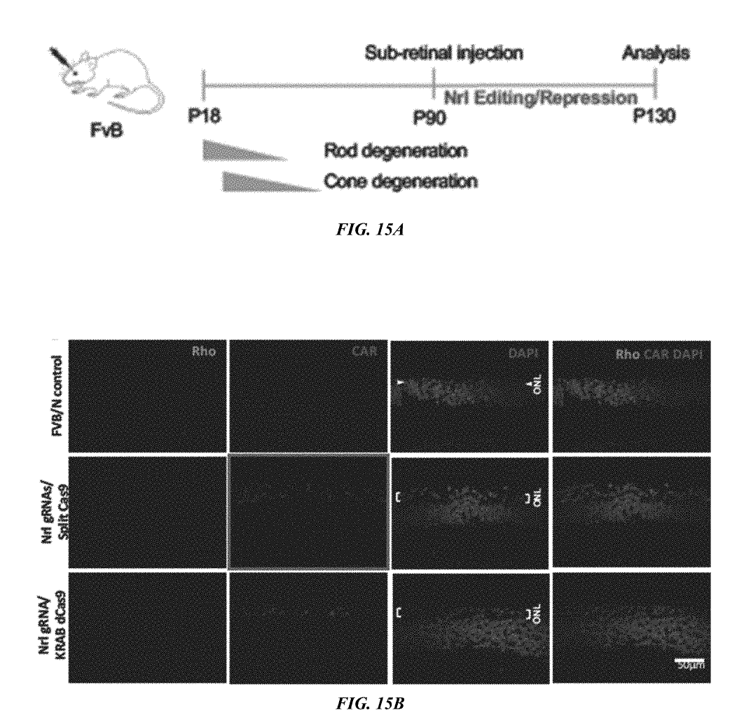

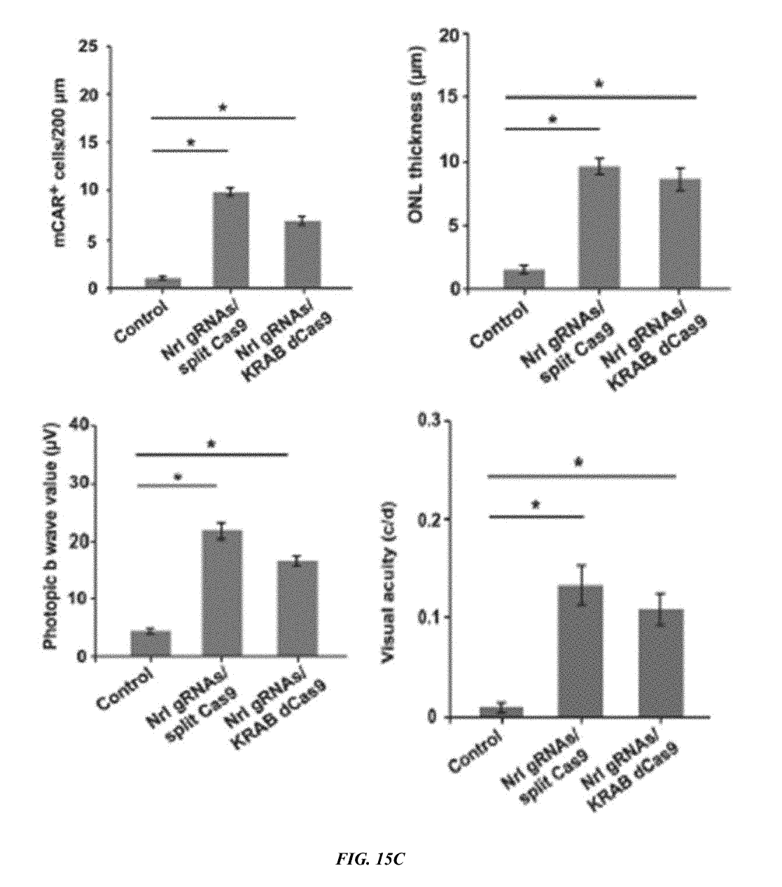

The Royal College of Surgeons (RCS) rat is a widely used animal model of inherited retinal degeneration called retinitis pigmentosa, a common cause of blindness in humans. A homozygous mutation in the Mertk gene, which harbors a 1.9 kb deletion from intron 1 to exon 2, results in defective phagocytic function of the retinal pigment epithelium (RPE), with consequent RPE and overlaying photoreceptor degeneration and blindness (FIG. 7A). Retinal degeneration in RCS rats can be evaluated by morphology and visual function testing via electroretinography (ERG). Morphological changes in the photoreceptor outer nuclear layer (ONL) degeneration appear as early as postnatal day 16 (P16) in RCS rats. To restore the retinal function of the Mertk gene in the eye, an AAV vector that can insert a functional copy of exon 2 of the Mertk into intron 1 via HITI (AAV-rMertk-HITI) was generated. For comparison a HDR AAV vector was also generated to restore the deleted 1.9 bp regions (AAV-rMertk-HDR) (FIG. 7B). The AAVs were injected in rat eyes at postnatal 3 weeks, and analyzed at 7-8 weeks (FIG. 7C). From DNA analysis, correct DNA knock-in in the AAV injected eye was detected (FIG. 7D, and FIG. 8). HITI-AAV injection led to a significant increase in Mertk mRNA expression levels and better preservation of the ONL thickness compared with untreated and HDR-AAV controls (FIGS. 7E & 7F). H&E staining confirmed an increased photoreceptor ONL in the injected eye. In contrast, untreated and HDR-AAV treated eyes had only one-two or sparsely distributed photoreceptor cell bodies in the ONL, The MERTK protein expression was also observed in the HITI-AAV, but not HDR-AAV injected eyes (FIG. 7G). To determine the effect of the treatment on retinal physiological function, ERG responses were tested at 4 weeks after injection (P50) to measure the electrical activity of rods and cones function (10 Hz flicker). Briefly, eyes of deeply anesthetized mice were dilated with 1% topical tropicamide. One active lens electrode was placed on each cornea, with a subcutaneously-placed ground needle electrode in the tail and reference electrodes subcutaneously in the head, approximately between the eyes. Light simulations were delivered with a xenon lamp in a Ganzfeld bowl and results were processed with software from Diagnosys. Photopic ERG was performed as published: following light adaption for 10 minutes at a background light of 30 cd/m², cone responses were elicited by a 34 cds/m² flash light with a low background light of 10 cd/m² and signals were averaged from 50 sweeps. All eyes treated with HITI-AAV exhibited significantly improved ERG b-wave responses (FIG. 7H). Similarly, 10 Hz flicker value, which measures cone response, was significantly improved and was more than 4-fold higher than that of the untreated eyes (FIG. 7I). These results demonstrate that AAV-HITI treatment is able to rescue and preserve retinal visual function in the RCS rat model.

### Example 6. Intraperitoneal Injections with AAV Encoding Cas9/CRISPR System Targeting Colon Cancer Cells

One or more viruses encoding Cas 9 and two guide RNAs targeting a gene that carries a mutation driving colon cancer are injected intraperitoneally into a subject with colon cancer. The gene is APC. Alternatively, the gene is MYH1, MYH2, MYH3, MLH1, MSH2, MSH6, PMS2, EPCAM, POLE1, POLD1, NTHL1, BMPR1A, SMAD4, PTEN or STK11. A colon biopsy is obtained four weeks later and compared to a colon biopsy obtained from the subject before treatment with the virus(es). The number of colon cancer cells in the biopsy sample obtained after treatment are fewer and small intestinal cells more numerous compared to that of the biopsy sample obtained before treatment. It is concluded that colon cancer cells have been reprogrammed to benign small intestinal cells.

### Example 7. Intravenous Injections with AAV Encoding Cas9/CRISPR System Targeting Lymphoma Cells

One or more viruses encoding Cas 9 and two guide RNAs targeting a gene that carries a mutation driving B cell lymphoma are injected intravenously into a subject with B cell lymphoma. The gene is C-MYC. Alternatively, the gene is CCND1, BCL2, BCL6, TP53, CDKN2A, or CD19. A blood sample is obtained four weeks later and compared to a blood sample obtained from the subject before treatment with the virus(es). The number of B cells in the blood sample obtained after treatment are fewer and macrophages more numerous compared to that of the blood sample obtained before treatment. It is concluded that B cell lymphoma cells have been reprogrammed to benign macrophages.

### Example 8. Intravenous Injections with AAV Encoding Cas9/CRISPR System Targeting T Cells for Immunotherapy

One or more viruses encoding Cas 9 and two guide RNAs targeting the PD-1 and/or PD-L1 checkpoint inhibitor encoding genes are injected intravenously into a patient with metastatic melanoma. Alternatively, the patient has another cancer such as metastatic ovarian cancer, metastatic renal cell carcinoma or non-small cell lung cancer. T cells are infected with the virus and the PD-1 encoding gene is inactivated, such that T cell numbers and response are maximized. Cancer cells of the patient expressing PD-L1 are infected also and PD-L1 is inactivated as well, reducing PD-L1 inhibition of T-cell activation and cytokine production, which normally provides immune escape to the cancer cell.

### Example 9. Split Cas9 Delivery Platform

CRISPR/Cas9-mediated targeted inactivation of NRL in the retina to effect in vivo rod to cone reprogramming was performed as follows. The adeno-associated viruses were chosen for gene transfer due to their mild immune response, long-term transgene expression, and favorable safety profile. To overcome their limited packaging capacity, a split-Cas9 system was used. The S. pyogenes Cas9(SpCas9) protein was split in to two parts using split-inteins. Each SpCas9 portion was fused to its corresponding split-intein moiety. Upon co-expression, the full SpCas9 protein was reconstituted. By utilizing two AAV vectors in this way (see FIG. 9), the residual packaging capacity of each vector accommodated a broad range of genome engineering functionalities, including multiplex targeting via single or dual-gRNA delivery and also AAV-CRISPR-Cas9-mediated targeted in vivo gene repression for in situ therapy.

### Example 10. Effectiveness of Dual Vector Delivery Using One or Two gRNAs

The dual-AAV vector approach was assessed for delivery of Cas9 and gRNAs targeting NRL. Constructs with either one or two gRNAs targeting NRL were designed in order to determine if targeting two sites by two gRNAs for the same gene have a higher targeting efficiency than by a single gRNA. Target sequences are shown in FIG. 10A with PAM sequence underlined. Further, to avoid repeat sequences in the AAV, thereby compromising vector stability and viral titers, a human U6 promoter and a mouse U6 promoter to drive each gRNA independently was used. Additional non-homologous tracrRNA was employed. A standard T7 Endonuclease 1 was used to quantify gene editing rates in mouse embryonic fibroblasts (MEFs). MEFs were co-transfected with split Cas9-Nrl vectors and T7E1 assay was carried out using genomic DNA (FIG. 10B). Arrows indicate cleaved DNA produced by T7E1 enzyme that is specific to heteroduplex DNA caused by genome editing. Mutation frequency was calculated from the proportion of cut bands intensity to total bands intensity. Gene targeting efficiency was improved with the dual-gRNA targeting strategy over a single gRNA method.

### Example 11. Inclusion of KRAB Transcription Repressor in Dual-Vector System

Transcription interference was effected through use of a KRAB transcription repressor. Building on the dual-AAV vector system described in Example 10, a KRAB transcription repressor was incorporated to the split-Cas9 system by fusing the KRAB repressor domain to the N-terminus of the Cas9 protein sequence (FIG. 11). This created a scar-free and potentially reversible approach for gene therapy, with minimized risk of mutagenesis due to inactivation of Cas9 nuclease activity.

### Example 12. Rod to Cone Cellular Reprogramming in Wild-Type and NRL-GFP Mice

AAV-gRNA/Cas9 or AAV-gRNA/KRAB-dCas9 targeting NRL were injected in to the subretinal space in wildtype mice postnatal day 7 (p′7) and sacrificed for histology at P30 (FIG. 12A). Both AAV2 capsid and tyrosine mutant Y444F were evaluated for transduction efficiency. The Y444F mutant vector showed enhanced retinal transduction over AA2 and was used in subsequent investigations. Retinas were flash-frozen, sectioned, and stained for cone markers, including cone arrestin (mCAR) and medium wavelength opsin (M-opsin). As shown in stained sections and with cell assays (FIGS. 12B-D), a reprogrammed photoreceptor phenotype was seen with Cas9-gRNAs. Cone-specific expression is visualized in the ONL as compared to Wild Type-Control. Quantitative RT-PCR (qRT-PCR) was used to measure the relative expression levels of rod or cone genes in reprogrammed retinas and controls. There was down-regulation of rod-specific genes with concomitant upregulation of cone-specific genes (FIG. 12E).

Transgenic NRL-GFP mice (wherein all rod photoreceptor cells are labelled) were injected subretinally with AAV-NRL gRNA/Cas9 as described (FIG. 12F). A significant increase in the number of mCAR positive cells and concomitant decrease in Nrl-GFP⁺ rod photoreceptors was seen (FIGS. 12G and 12H). Many morphologically cone-like cells were noted in the inner aspect of the inner nuclear layer, reminiscent of horizontal cells (HC) in the wild-type retinas (FIG. 12I). Additionally, it was detected that these cells expressed both a cone marker, m-CAR, and an HC marker, Calbindin, (FIG. 12J), indicating that horizontal cells also maintain the potential to undergo cone-like cell reprogramming. It is concluded that rods have been reprogrammed to cone-like cells.

### Example 13

NRL in rd10 mice, a model for autosomal recessive RP, was targeted. These rd10 mice carry a spontaneous mutation of the rod-phosphodiesterase gene, and exhibit rapid rod degeneration starting around P18. By P60, rods are no longer visible, with accompanying cone photoreceptor degeneration. To assess if conversion of rods to cones is sufficient to reverse retinal degeneration and rescue visual function, AAV-gRNA/Cas9 or AAV-gRNA/KRAB-dCas9 was injected in to rd10 mice at P7. The effect of such treatment on cone physiological function and visual acuity was determined by measuring electroretinography (ERG) responses and optic kinetic nystagmus (OKN) to quantify cone photoreceptor activity (photopic response) and visual acuity 6 weeks after injection (P60) (FIG. 13A). OKN was measured, briefly, by creating a virtual reality chamber with four computer monitors surrounding a platform upon which the test animal was placed. After allowing the animal to acclimate to the test conditions, a virtual cylinder, covered with a vertical sine wave grating, was projected onto the monitors. The virtual stripe cylinder was set up at the highest level of contrast (100%, black 0, white 255, illuminated from above 250 cd/m²) with the number of stripes starting from 4 per screen (2 black and 2 white). The test began with 1 min of clockwise rotation at a speed of 13, followed by 1 min of counterclockwise rotation. A video camera situated above the animal allowed an unbiased observer to track and record head movements. Data was measured by cycles/degree (c/d) and expressed as mean±S.D., with comparison using t-test statistical analysis. A p-value<0.05 was considered statistically significant. All eyes treated with AAV-gRNA/Cas9 or KRAB-dCas9 had improved cone function and visual function, as indicated by significant improvement in photopic b-wave value and acuity (FIGS. 13B-C). Further, a number of mCAR positive cells and M-opsin positive cells were observed on histological analysis of AAV-NRL gRNAs/Cas9 or KRAB-dCAS9-treated rd10 retinas (FIGS. 13D-G), consistent with findings of improvement in visual function. While untreated eyes had only sparsely distributed photoreceptor cell nuclei in the ONL, AAV-gRNA/Cas9 or AAV-gRNA/KRAB-dCas9 treated eyes had 3˜5 layers of ONL (FIG. 13D), indicating treatment prevented photoreceptor degeneration and preserved ONL.

### Example 14 Generation of Cone-Like Cells in Late/End Stage Disease

AAV-gRNA/Cas9 or AAV-gRNA/KRAB-dCas9 was injected subretinally at P60 (FIG. 14A) in to rd10 mice in which there were no viable photoreceptors and a non-recordable ERG. All eyes treated with AAV-gRNA/Cas9 or AAV-gRNA/KRAB-dCas9 had improved cone function and visual function, as indicated by significant improvement in photopic b-wave value and visual acuity (FIG. 14B-C) with concomitant increase in a number of cone mCAR positive cells. Co-localized Calbindin expression in a significant portion of cone Opsin⁺ cells were observed in all eyes treated with AAV-gRNA/Cas9 or AAV-gRNA/KRAB-dCas9 in newborn and adult rd10 mice (FIG. 14D). It is concluded that interneuron to cone reprogramming can be applied to RP gene therapy at a late/end stage in which rod and cone photo receptors have been substantially degenerated and lost.

### Example 15. Recovering Retinal Function in 3-Month Olf FvB Retinal Degeneration Mice

FVB/N mice, with a homozygous mutation for Pde6b^{rdl} encoding the B-subunit of cGMP phosphodiesterase (PDE), show heritable autosomal recessive retinal degeneration which is characterized by rapid initial loss of rod photoreceptors and subsequent loss of cone photoreceptors by p35. Such mice were injected subretinally at P60 (FIG. 15A) with AAV-gRNA/KRAB-dCAS9. Histology analysis was performed as in previous examples. AAV-gRNA/KRAB-dCAS9-treated retinas showed emergence of mCAR⁺ cells with significantly improved photopic b-wave values and visual acuity, showing improved visual function (FIGS. 15 B-C). It is concluded that CRISPR/Cas-9-mediated cellular reprogramming described herein is a gene and mutation-independent therapy.

What is claimed is:

1. A method of re-programming a cell from a first cell type to a second cell type, comprising contacting the cell with:
a) a guide RNA that hybridizes to a target site of a gene, wherein the gene encodes a protein that contributes to a cell type specific function of the cell; and
b) a Cas nuclease, or polynucleotide encoding the Cas nuclease, wherein the Cas nuclease cleaves a strand of the gene at the target site,
wherein cleaving the strand modifies expression of the gene such that the cell can no longer perform the cell type specific function, thereby re-programming the cell to the second cell type.

2. The method of claim 1, wherein the gene comprises a mutation that causes a detrimental effect in the first cell type, wherein the detrimental effect is selected from senescence, apoptosis, lack of differentiation, and aberrant cellular proliferation.

3. The method claim 1, wherein the gene encodes a transcription factor.

4. The method of claim 1, wherein the gene comprises a mutation that causes a detrimental effect in the first cell type, wherein the detrimental effect is selected from senescence, apoptosis, lack of differentiation, and aberrant cellular proliferation.

5. The method claim 1, wherein the gene encodes a transcription factor.

6. The method of claim 1, wherein the gene is selected from NRL, NR2E3, GNAT1, ROR beta, OTX2, CRX and THRB.

7. The method of claim 1, wherein the cell is an eye cell.

8. The method of claim 1, wherein the cell is a retinal cell.

9. The method of claim 1, wherein the first cell type is a rod and the second cell type is a cone.

10. The method of claim 1, wherein the cell type specific function is night vision or color vision.

11. The method of claim 1, wherein the first cell type is a colon cancer cell and the second cell type is a benign intestinal or colon cell, and wherein the gene is selected from APC, MYH1, MYH2, MYH3, MLH1, MSH2, MSH6, PMS2, EPCAM, POLE1, POLD1, NTHL1, BMPR1A, SMAD4, PTEN, and STK11.

12. The method of claim 1, wherein the first cell type is a malignant B cell and the second cell type is a benign macrophage, and wherein the gene is selected from C-MYC, CCND1, BCL2, BCL6, TP53, CDKN2A, and CD19.

13. The method of claim 1, wherein the first cell type is a neuron and the second cell type is a glial cell, and wherein the gene is selected from APP and MAPT.

14. The method of claim 1, wherein the first cell type is a glial cell and the second cell type is a dopamine producing neuron, and wherein the gene is selected from SNCA, LRRK2, PARK2, PARK7, and PINK1.

15. A method of treating an eye condition comprising administering to a subject in need thereof:
a) a first guide RNA that hybridizes to a target site of a gene in a first type of cell, wherein the gene encodes a protein that contributes to a first function of the first type of cell; and
b) a Cas nuclease that cleaves a strand of the gene at the target site,
wherein cleaving the strand modifies expression of the gene such that the first type of cell is switched from a first type of cell to a second type of cell, wherein a resulting presence or increase in the second type of cell improves the eye condition.

16. The method of claim 15, wherein modifying expression of the gene comprises reducing expression of the gene in the first type of cell by at least about 90%.

17. The method of claim 15, wherein modifying expression of the gene comprises editing the gene, wherein the editing results in production of no protein from the gene or a non-functional protein from the gene.

18. The method of claim 15, wherein the first type of eye cell is a rod and the second type of eye cell is a cone.

19. The method of claim 15, wherein the eye condition is retinal degeneration, retinitis pigmentosa or macular degeneration.

20. The method of claim 15, wherein the gene is selected from NRL, NR2E3, GNAT1, ROR beta, OTX2, CRX and THRB.

21. A method of treating an eye condition in a subject in need thereof with a re-programmed cell, wherein the re-programmed cell is produced by the method of claim 1.

22. The method of claim 21, wherein the re-programmed cell is autologous to the subject.

23. The method of claim 21, wherein the condition is selected from macular degeneration, retinitis pigmentosa, and glaucoma.

24. The method of claim 21, wherein the gene is selected from NRL, NR2E3, GNAT1, ROR beta, OTX2, CRX and THRB.

25. A pharmaceutical composition for treating a condition of an eye in a subject, comprising:
a) a Cas nuclease or a polynucleotide encoding the Cas nuclease; and
b) at least one guide RNA that is complementary to a portion of a gene selected from NRL, NR2E3, GNAT1, ROR beta, OTX2, CRX and THRB.

26. The pharmaceutical composition of claim 25, wherein the polynucleotide encoding the Cas protein and the at least one guide RNA are present in at least one viral vector.

27. The pharmaceutical composition of claim 25, wherein the polynucleotide encoding the Cas protein or the at least one guide RNA are present in a liposome.

28. The pharmaceutical composition of claim 25, wherein the at least one guide RNA targets the Cas protein to a sequence comprising any one of SEQ ID NOS.: 1-4.

29. The pharmaceutical composition of claim 25, wherein the pharmaceutical composition is formulated as a liquid for administration with an eye dropper.

30. The pharmaceutical composition of claim 25, wherein the pharmaceutical composition is formulated as a liquid for intravitreal administration.


SEQUENCE LISTING (19 sequences)

SEQ ID NO: 1 - DNA, 23 bp, Unknown
  Description of Unknown target sequence
gagccttctgagggccgatctgg

SEQ ID NO: 2 - DNA, 23 bp, Unknown
  Description of Unknown target sequence
gtatggtgtggagcccaacgagg

SEQ ID NO: 3 - DNA, 23 bp, Unknown
  Description of Unknown target sequence
ggcctggcactgattgcgatggg

SEQ ID NO: 4 - DNA, 23 bp, Unknown
  Description of Unknown target sequence
aggcctggcactgattgcgatgg

SEQ ID NO: 5 - protein, 6 aa, Artificial Sequence
  Description of Artificial Sequence Synthetic 6xHis tag
HHHHHH

SEQ ID NO: 6 - DNA, 23 bp, Unknown
  Description of Unknown target sequence
ggctgctggtggtctacccttgg

SEQ ID NO: 7 - DNA, 23 bp, Unknown
  Description of Unknown target sequence
ggtagaccaccagcagcctaagg

SEQ ID NO: 8 - DNA, 39 bp, Artificial Sequence
  Description of Artificial Sequence Synthetic primer
taatacgactcactatagggacccagaggttgagtcctt

SEQ ID NO: 9 - DNA, 20 bp, Artificial Sequence
  Description of Artificial Sequence Synthetic primer
aaaagcaccgactcggtgcc

SEQ ID NO: 10 - DNA, 20 bp, Artificial Sequence
  Description of Artificial Sequence Synthetic primer
cccagaggttcttcgaatcc

SEQ ID NO: 11 - DNA, 21 bp, Artificial Sequence
  Description of Artificial Sequence Synthetic primer
tcattcgtctgtttcccattc

SEQ ID NO: 12 - DNA, 21 bp, Artificial Sequence
  Description of Artificial Sequence Synthetic primer
ctgcctattggtctattttcc

SEQ ID NO: 13 - DNA, 19 bp, Artificial Sequence
  Description of Artificial Sequence Synthetic primer
actcagtgtggcaaaggtg

SEQ ID NO: 14 - DNA, 24 bp, Artificial Sequence
  Description of Artificial Sequence Synthetic probe
cccagaggttcttcgaatcctttg

SEQ ID NO: 15 - DNA, 22 bp, Artificial Sequence
  Description of Artificial Sequence Synthetic probe
cttggacccagaggttgagtcc

SEQ ID NO: 16 - DNA, 54 bp, Artificial Sequence
  Description of Artificial Sequence Synthetic primer
tcgtcggcagcgtcagatgtgtataagagacagctgcctattggtctattttcc

SEQ ID NO: 17 - DNA, 53 bp, Artificial Sequence
  Description of Artificial Sequence Synthetic primer
gtctcgtgggctcggagatgtgtataagagacagactcagtgtggcaaaggtg

SEQ ID NO: 18 - DNA, 23 bp, Unknown
  Description of Unknown target sequence
tccctgtattcagaacaaggggg

SEQ ID NO: 19 - DNA, 23 bp, Unknown
  Description of Unknown target sequence
agtcactgtcagaaccagaaagg